(19) United States
(12) Patent Application Publication
(10) Pub. No.: US 2025/0066304 A1
(43) Pub. Date: Feb. 27, 2025

(54) ANTIDEGRADANT COMPOUNDS AND USES THEREOF

(71) Applicant: FLEXSYS AMERICA L.P., Akron, OH (US)

(72) Inventors: Judicael Jacques CHAPELET, Copley, OH (US); Malik Hani AL-AFYOUNI, Akron, OH (US); Donald L. FIELDS, JR., Copley, OH (US); Frederick IGNATZ-HOOVER, Elyria, OH (US); Jonathan PENNEY, Cuyahoga Falls, OH (US); Hiu Ching WONG, Tallmadge, OH (US)

(21) Appl. No.: 18/938,965

(22) Filed: Nov. 6, 2024

Related U.S. Application Data
(63) Continuation-in-part of application No. PCT/US2023/021205, filed on May 5, 2023.
(60) Provisional application No. 63/338,959, filed on May 6, 2022.
(60) Provisional application No. 63/379,392, filed on Oct. 13, 2022.

(51) Int. Cl. C07D 215/06; B29D 30/54; C07C 211/50; C07C 211/51; C07C 215/76; C07C 323/20; C07D 215/12; C07D 215/36; C07D 307/52; C08K 5/1535; C08K 5/18; C08K 5/3432; C08L 7/00; C08L 9/00; C08L 9/06; C10L 1/223; C10L 1/232; C10L 1/24; C10M 133/12; C10M 133/40; C10M 135/28; C10M 169/04

(52) U.S. Cl. CPC C07D 215/06; C07C 211/50; C07C 211/51; C07C 215/76; C07C 323/20; C07D 215/12; C07D 215/36; C07D 307/52; C08K 5/1535; C08K 5/18; C08K 5/3432; C08L 7/00; C08L 9/00; C08L 9/06; C10L 1/223; C10L 1/232; C10L 1/2443; C10M 133/12; C10M 133/40; C10M 135/28; C10M 169/04; B29D 30/54; C08K 2201/012; C08L 2205/025; C10M 2203/003; C10M 2215/26; C10M 2215/30; C10M 2219/09

(57) ABSTRACT

The present disclosure provides compounds represented by Formula (I):

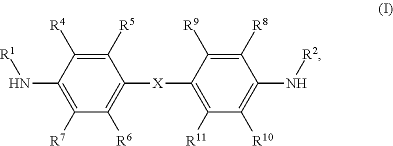

or salts or solvates thereof, wherein R¹, R², R⁴, R⁵, R⁶, R⁷, R⁸, R⁹, R¹⁰, R¹¹, and X are as defined in the specification. The present disclosure also provides compositions, vulcanized elastomeric articles, lubricant compositions, combustible fuel compositions, and fuel additive compositions comprising a compound disclosed herein. The present disclosure also provides processes for preparing the compositions and vulcanized elastomeric articles described herein. The present disclosure also provides a process for retreading tires using a composition described herein. The present disclosure also provides kits comprising a composition described herein.

### BACKGROUND

### Field

The present disclosure provides compounds with antidegradant, e.g., antiozonant, antioxidant and/or antifatigue, properties that are useful additives for vulcanized rubber articles, compositions comprising elastomers, lubricants, fuels, and other compositions which require such properties or in compositions which are themselves useful as compositions to impart such properties.

### Background

Many materials such as plastics, elastomers, elastomeric products (tires, belts, hoses, bushings, mounts, vibration isolators, etc.), lubricants and petroleum products (such as hydraulic fluids, oils, fuels and oil/fuel additives for automotive and aviation applications) are prone to degradation upon prolonged exposure to light, heat, oxygen, ozone, repetitive mechanical actions and the like. Accordingly, compounds and compositions demonstrating antidegradant efficacy are well known in the art. For example, U.S. Pat. No. 8,987,515 discloses an aromatic polyamine useful in inhibiting oxidative degradation particularly in lubricant compositions. U.S. Patent Application Publication No. 2014/0316163 discloses antioxidant macromolecules with purported improved solubility in many commercially available oils and lubricants.

Antidegradants useful in the manufacture of articles formed from elastomers, plastics and the like require a very specific combination of qualities that can be difficult to achieve. While the antidegradants must obviously have commercially acceptable efficacy, they must also exhibit that efficacy over prolonged periods of time associated with use of the article, particularly at exposed surfaces of the article where degradation from environmental factors such as light, oxygen and ozone primarily occurs. Just as important to the protection of surface exposed components, efficacy in protecting imbedded components of composite materials from the effects of oxidative aging and repetitive mechanical action are critically important. The antidegradants must achieve these results while not negatively impacting other additives' efficacy or desirable characteristics in the final article. Further, antidegradants which provide or improve the mechanical fatigue life after an article has been in service, aged oxidatively or by exposure to ozone are highly valued since these will inherently improve the useful mechanical service life of article. Consequently, elastomeric articles which undergo repeated mechanical flexure, extension, or compression during service would greatly benefit from such a discovery.

Articles formed from general purpose elastomers such as natural rubber, in particular tires, are especially prone to degradation from both oxygen and ozone. As discussed in U.S. Pat. No. 2,905,654, the effect on rubber from degradation by oxygen is different from the effect from degradation from ozone; however, both effects can be detrimental to tire performance, appearance and life expectancy. Fatigue and crack propagation are also issues of specific concern, in particular for steel belt edge areas and tire sidewalls which are subject to significant stresses and stretching forces while flexed whether inflated, partially inflated and throughout the service life of the tire. U.S. Pat. No. 8,833,417 describes an antioxidant system that purportedly increases long-term resistance to fatigue and crack propagation over the known antioxidants discussed immediately below.

Materials with antidegradant efficacy are well known in the art for use in tire applications and are commercially available. For example, N,N′-disubstituted-paraphenylenediamines such as those sold under the trademark Santoflex® are generally favored by many tire manufacturers for this purpose. EP 3147321 A1 discloses rubber compositions, tires, amine compounds, and anti-aging agents, and in particular, a rubber composition that is said to be suitable for use in tread rubber or sidewall rubber of a tire.

### BRIEF SUMMARY

The present disclosure provides compounds that are useful as antidegradants, e.g., antiozonants, and/or antioxidants, and/or as additives in lubricants or combustible fuels, represented by any one of Formulae I-VI, (IVa), (Va), or (VIa), below, collectively referred to herein as “Compounds of the Disclosure” or individually as a “Compound of the Disclosure.”

The present disclosure also provides compositions comprising:
(i) a Compound of the Disclosure; and
(ii) one or more elastomers; or
(iii) one or more fillers; or
(iv) one or more rubber chemicals; or
(v) one or more plasticizers; or
(vi) a second antidegradant; or
(vii) a combination of one or more elastomers, one or more fillers, one or more rubber chemicals, one or more plasticizers, and/or a second antidegradant, collectively referred to herein as “Compositions of the Disclosure” or individually as a “Composition of the Disclosure.”

The present disclosure also provides Compositions of the Disclosure comprising a Compound of the Disclosure and one or more elastomers.

The present disclosure also provides Compositions of the Disclosure comprising a Compound of the Disclosure and one or more fillers.

The present disclosure also provides Compositions of the Disclosure comprising a Compound of the Disclosure and one or more rubber chemicals.

The present disclosure also provides Compositions of the Disclosure comprising a Compound of the Disclosure and one or more plasticizers.

The present disclosure also provides Compositions of the Disclosure comprising a Compound of the Disclosure and a second antidegradant.

The present disclosure also provides Compositions of the Disclosure comprising a Compound of the Disclosure and one or more carriers.

The present disclosure also provides processes for preparing a composition comprising a Compound of the Disclosure and one or more carriers, the process comprising admixing the compound and the one or more carriers.

The present disclosure also provides vulcanized elastomeric articles comprising a Compound of the Disclosure.

The present disclosure also provides vulcanized elastomeric articles prepared using a composition described herein, e.g, Compositions of the Disclosure or compositions comprising a Compound of the Disclosure and one or more carriers.

The present disclosure also provides a process for preparing the vulcanized elastomeric articles described herein, the process comprising:
(a) forming a composition described herein into a formed shape; and
(b) vulcanizing the formed shape to provide a vulcanized elastomeric article.

The present disclosure also provides lubricant compositions comprising a lubricant and a Compound of the Disclosure.

The present disclosure also provides combustible fuel compositions comprising a combustible fuel and a Compound of the Disclosure.

The present disclosure also provides fuel additive compositions comprising a fuel additive and a Compound of the Disclosure.

The present disclosure also provides a process for retreading tires, the process comprising:
(a) applying a composition described herein to a tire;
(b) disposing a pre-vulcanized tread around the tire;
(c) disposing a curing envelope around the tire; and
(d) vulcanizing the tire.

The present disclosure also provides kits comprising a composition described herein and instructions for using the composition in a vulcanizable elastomeric composition.

The present disclosure also provides kits comprising a composition described herein and instructions for using the composition to prepare a vulcanized elastomeric article.

Additional embodiments and advantages of the disclosure will be set forth, in part, in the description that follows, and will flow from the description, or can be learned by practice of the disclosure. The embodiments and advantages of the disclosure will be realized and attained by means of the elements and combinations particularly pointed out in the appended claims.

It is to be understood that both the foregoing summary and the following detailed description are exemplary and explanatory only, and are not restrictive of the invention as claimed.

### BRIEF DESCRIPTION OF THE DRAWINGS

FIG. 1(a) is a line graph depicting a ¹H nuclear magnetic resonance (NMR) spectrum of Compound 1 in d₆-dimethylsulfoxide (d₆-DMSO).

FIG. 1(b) is a line graph depicting the ¹³C NMR spectrum of Compound 1 in d₆-DMSO.

FIG. 2(a) is a line graph depicting the ¹H NMR spectrum of Compound 2 in d-chloroform (CDCl₃).

FIG. 2(b) is a line graph depicting the ¹³C NMR spectrum of Compound 2 in CDCl₃.

FIG. 3(a) is a line graph depicting the ¹H NMR spectrum of Compound 3 in d₆-DMSO.

FIG. 3(b) is a line graph depicting the ¹³C NMR spectrum of Compound 3 in d₆-DMSO.

FIG. 4(a) is a line graph depicting the ¹H NMR spectrum of Compound 153 in d₆-DMSO.

FIG. 4(b) is a line graph depicting the ¹³C NMR spectrum of Compound 153 in d₆-DMSO.

FIG. 5(a) is a line graph depicting the ¹H NMR spectrum of Compound 154 in CDCl₃.

FIG. 5(b) is a line graph depicting the ¹³C NMR spectrum of Compound 154 in d₈-tetrahydrofuran.

FIGS. 6(a)-(c) are line graphs showing force retention as a function of time for sidewall compounds comprising Compounds 1, 2, or 3 under (a) static, (b) intermittent, and (c) dynamic conditions.

FIG. 7 is a line graph showing force retention as a function of time for sidewall compounds comprising Compounds of the Disclosure under dynamic conditions.

FIG. 8 is a line graph showing force retention as a function of time for tread compounds comprising Compounds of the Disclosure under dynamic conditions.

### DETAILED DESCRIPTION

In one embodiment, Compounds of the Disclosure are compounds having Formula (I):

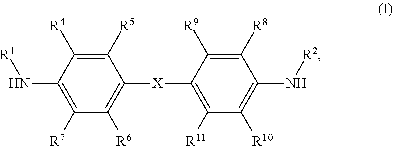

or a salt or solvate thereof, wherein:
R¹ is selected from the group consisting of C₁-C₁₂ alkyl, —CHR^{1a}R^{1b}, C₃-C₆ cycloalkyl, and optionally substituted phenyl; or
R¹ and R⁴ taken together with the nitrogen and carbon atoms, respectively, to which they are attached form a saturated or partially saturated optionally substituted 5- or 6-membered heterocyclo;
R^{1a} is selected from the group consisting of optionally substituted phenyl, C₃-C₆ cycloalkyl, and optionally substituted 5- or 6-membered heteroaryl;
R^{1b} is selected from the group consisting of hydrogen and C₁-C₆ alkyl;
R² is selected from the group consisting of C₁-C₁₂ alkyl, —CHR^{2a}R^{2b}, C₃-C₆ cycloalkyl, and optionally substituted phenyl; or
R² and R⁸ taken together with the nitrogen and carbon atoms, respectively, to which they are attached form a saturated or partially saturated optionally substituted 5- or 6-membered heterocyclo;
R^{2a} is selected from the group consisting of optionally substituted phenyl, C₃-C₆ cycloalkyl, and optionally substituted 5- or 6-membered heteroaryl;
R^{2b} is selected from the group consisting of hydrogen and C₁-C₆ alkyl;
X is selected from the group consisting of —CHR^{3a}—, —NR^{3b}—, —O—, and —S—;
R^{3a} is selected from the group consisting of hydrogen, C₁-C₁₂ alkyl, C₂-C₆ alkenyl, C₃-C₆ cycloalkyl, optionally substituted phenyl, optionally substituted 5- or 6-membered heterocyclo, and optionally substituted 5- or 6-membered heteroaryl;
R^{3b} is selected from the group consisting of hydrogen, C₁-C₁₂ alkyl, C₃-C₆ cycloalkyl, optionally substituted phenyl, optionally substituted 5- or 6-membered heterocyclo, and optionally substituted 5- or 6-membered heteroaryl;
R⁴, R⁵, R⁶, and R⁷ are independently selected from the group consisting of hydrogen, halogen, C₁-C₆ alkyl, C₁-C₆ alkoxy, and C₁-C₆ alkylthio; or
R¹ and R⁴ taken together with the nitrogen and carbon atoms, respectively, to which they are attached form a saturated or partially saturated optionally substituted 5- or 6-membered heterocyclo; and
R⁵, R⁶, and R⁷ are independently selected from the group consisting of hydrogen, halogen, C₁-C₆ alkyl, C₁-C₆ alkoxy, and C₁-C₆ alkylthio; and
R⁸, R⁹, R¹⁰, and R¹¹ are independently selected from the group consisting of hydrogen, halogen, C₁-C₆ alkyl, C₁-C₆ alkoxy, and C₁-C₆ alkylthio; or
R² and R⁸ taken together with the nitrogen and carbon atoms, respectively, to which they are attached form a saturated or partially saturated optionally substituted 5- or 6-membered heterocyclo; and
   R⁹, R¹⁰, and R¹¹ are independently selected from the group consisting of hydrogen, halogen, C₁-C₆ alkyl, C₁-C₆ alkoxy, and C₁-C₆ alkylthio.

In some embodiments, Compounds of the Disclosure are compounds having Formula (I), wherein R^{3a} is selected from the group consisting of hydrogen, C₁-C₁₂ alkyl, C₃-C₆ cycloalkyl, optionally substituted phenyl, optionally substituted 5- or 6-membered heterocyclo, and optionally substituted 5- or 6-membered heteroaryl;

In some embodiments, Compounds of the Disclosure are compounds having Formula (I), wherein X is —NR^{3b}—.

In some embodiments, Compounds of the Disclosure are compounds having Formula (I), wherein X is —O—.

In some embodiments, Compounds of the Disclosure are compounds having Formula (I), wherein X is —O— and R¹ and R² are independently selected from the group consisting of —CHR^{1a}R^{1b}, C₃-C₆ cycloalkyl, and optionally substituted phenyl.

In some embodiments, Compounds of the Disclosure are compounds having Formula (I), wherein X is —S—.

In some embodiments, Compounds of the Disclosure are compounds having Formula (I), wherein X is —S— and R¹ and R² are independently selected from the group consisting of —CHR^{1a}R^{1b}, C₃-C₆ cycloalkyl, and optionally substituted phenyl.

In some embodiments, Compounds of the Disclosure are compounds having Formula (I), wherein X is —CHR^{3a}—.

In some embodiments, Compounds of the Disclosure are compounds having Formula (I), wherein if X is —CH₂—, then:
(i) R¹ is selected from the group consisting of nonyl, decyl, undecyl, dodecyl, —CHR^{1a}R^{1b} and C₃-C₅ cycloalkyl; or
(ii) R¹ and R⁴ taken together with the nitrogen and carbon atoms, respectively, to which they are attached form a saturated or partially saturated optionally substituted 5- or 6-membered heterocyclo;
R^{1a} is selected from the group consisting of substituted phenyl, C₃-C₆ cycloalkyl, and optionally substituted 5- or 6-membered heteroaryl;
R^{1b} is selected from the group consisting of hydrogen and C₁-C₆ alkyl;
R² is selected from the group consisting of nonyl, decyl, undecyl, dodecyl, —CHR^{2a}R^{2b} and C₃-C₅ cycloalkyl; or
(iii) R² and R⁸ taken together with the nitrogen and carbon atoms, respectively, to which they are attached form a saturated or partially saturated optionally substituted 5- or 6-membered heterocyclo;
R^{2a} is selected from the group consisting of substituted phenyl, C₃-C₆ cycloalkyl, and optionally substituted 5- or 6-membered heteroaryl; and
R^{2b} is selected from the group consisting of hydrogen and C₁-C₆ alkyl.

In some embodiments, Compounds of the Disclosure are compounds having Formula (I), wherein if X is —CHCH₃—, then:
(i) R¹ is selected from the group consisting of methyl, ethyl, propyl, butyl, tert-butyl, pentyl, hexyl, heptyl, octyl, nonyl, decyl, undecyl, dodecyl, —CHR^{1a}R^{1b}, C₃-C₆ cycloalkyl, and optionally substituted phenyl; or
(ii) R¹ and R⁴ taken together with the nitrogen and carbon atoms, respectively, to which they are attached form a saturated or partially saturated optionally substituted 5- or 6-membered heterocyclo; and
R² is selected from the group consisting of methyl, ethyl, propyl, butyl, tert-butyl, pentyl, hexyl, heptyl, octyl, nonyl, decyl, undecyl, dodecyl, —CHR^{2a}R^{2b}, C₃-C₆ cycloalkyl, and optionally substituted phenyl; or
(iii) R² and R⁸ taken together with the nitrogen and carbon atoms, respectively, to which they are attached form a saturated or partially saturated optionally substituted 5- or 6-membered heterocyclo.

In some embodiments, Compounds of the Disclosure are compounds having Formula (I), wherein if X is —O—, then:
(i) R¹ is selected from the group consisting of methyl, ethyl, propyl, butyl, tert-butyl, pentyl, hexyl, heptyl, octyl, nonyl, decyl, undecyl, dodecyl, —CHR^{1a}R^{1b}, C₃-C₆ cycloalkyl, and optionally substituted phenyl; or
(ii) R¹ and R⁴ taken together with the nitrogen and carbon atoms, respectively, to which they are attached form a saturated or partially saturated optionally substituted 5- or 6-membered heterocyclo; and
R² is selected from the group consisting of methyl, ethyl, propyl, butyl, tert-butyl, pentyl, hexyl, heptyl, octyl, nonyl, decyl, undecyl, dodecyl, —CHR^{2a}R^{2b}, C₃-C₆ cycloalkyl, and optionally substituted phenyl; or
(iii) R² and R⁸ taken together with the nitrogen and carbon atoms, respectively, to which they are attached form a saturated or partially saturated optionally substituted 5- or 6-membered heterocyclo.

In some embodiments, Compounds of the Disclosure are compounds having Formula (I), wherein if X is —S—, then:
(i) R¹ is selected from the group consisting of methyl, ethyl, propyl, butyl, tert-butyl, pentyl, hexyl, heptyl, nonyl, decyl, undecyl, dodecyl, —CHR^{1a}R^{1b}, C₃-C₆ cycloalkyl, and optionally substituted phenyl; or
(ii) R¹ and R⁴ taken together with the nitrogen and carbon atoms, respectively, to which they are attached form a saturated or partially saturated optionally substituted 5- or 6-membered heterocyclo; and
R² is selected from the group consisting of methyl, ethyl, propyl, butyl, tert-butyl, pentyl, hexyl, heptyl, nonyl, decyl, undecyl, dodecyl, —CHR^{2a}R^{2b}, C₃-C₆ cycloalkyl, and optionally substituted phenyl; or
(iii) R² and R⁸ taken together with the nitrogen and carbon atoms, respectively, to which they are attached form a saturated or partially saturated optionally substituted 5- or 6-membered heterocyclo.

In some embodiments, Compounds of the Disclosure are compounds having Formula (I), wherein if X is —CH₂—, then R¹ and R² are not sec-butyl, —CH(CH₃)CH₂CH(CH₃)CH₃, —CH(CH₃)CH₂CH₂CH(CH₃)CH₃, —CH(CH₃)(CH₂)₅CH₃, cyclohexyl, phenyl, 2-methylphenyl, 3-methylphenyl, 4-methylphenyl, 4-aminophenyl, 4-(isopropylamino)phenyl, 4-((4-methylpentan-2-yl)amino)phenyl, 4-((5-methylhexan-2-yl)amino)phenyl, 4-((octan-2-yl)amino)phenyl, or 4-(cyclohexylamino)phenyl.

In some embodiments, Compounds of the Disclosure are compounds having Formula (I), wherein if X is —C(CH₃)₂—, then R¹ and R² are not sec-butyl.

In some embodiments, Compounds of the Disclosure are compounds having Formula (I), wherein if X is —CH₂—, the R¹ and R² are not —CH₂(CH₂CH₃)CH₂CH₂CH₂CH₃, —CH(CH₂CH₃)CH₂CH₃, 4-methylcyclohexyl, 4-(tert-butyl)cyclohexyl, 3,5,5-trimethylcyclohexyl, 3,5,5-trimethylcyclohex-2-eneyl, 1-phenylethyl, or benzyl.

In some embodiments, Compounds of the Disclosure are compounds having Formula (I), wherein if X is —CH₂—, then R¹ and R² are not methyl, ethyl, propyl, isopropyl, or butyl.

In some embodiments, Compounds of the Disclosure are compounds having Formula (I), wherein if X is —CH₂— and R⁵ and R⁹ are methyl, then R¹ and R² are not sec-butyl.

In some embodiments, Compounds of the Disclosure are compounds having Formula (I), wherein if X is —CH₂— and R⁴ and R⁸ are methyl, then R¹ and R² are not isopropyl or sec-butyl.

In some embodiments, Compounds of the Disclosure are compounds having Formula (I), wherein if X is —O—, then R¹ and R² are not isopropyl or sec-butyl.

In some embodiments, Compounds of the Disclosure are compounds having Formula (I), wherein if X is —S—, then R¹ and R² are not isopropyl, sec-butyl, or —CH(CH₃)(CH₂)₅CH₃.

In another embodiment, Compounds of the Disclosure are compounds having Formula (II):

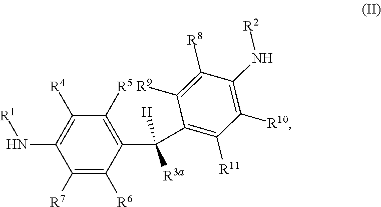

or a salt or solvate thereof, wherein R^{3a} is selected from the group consisting of C₁-C₁₂ alkyl, C₂-C₆ alkenyl, C₃-C₆ cycloalkyl, optionally substituted phenyl, and optionally substituted 5- or 6-membered heteroaryl; and R¹, R², R⁴, R⁵, R⁶, R⁷, R⁸, R⁹, R¹⁰, and R¹¹ are as defined in connection with Formula (I),
with the proviso that

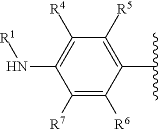

does not equal

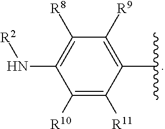

In another embodiment, Compounds of the Disclosure are compounds having Formula (III):

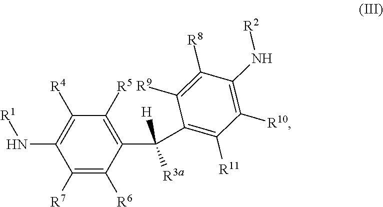

or a salt or solvate thereof, wherein R^{3a} is selected from the group consisting of C₁-C₁₂ alkyl, C₂-C₆ alkenyl, C₃-C₆ cycloalkyl, optionally substituted phenyl, and optionally substituted 5- or 6-membered heteroaryl; and R¹, R², R⁴, R⁵, R⁶, R⁷, R⁸, R⁹, R¹⁰, and R¹¹ are as defined in connection with Formula (I),
with the proviso that

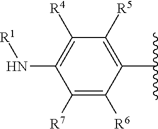

does not equal

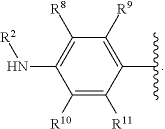

In some embodiments, Compounds of the Disclosure are compounds of any one of Formulae (I)—(III), wherein R¹ and R² are independently selected from the group consisting of methyl, ethyl, propyl, isopropyl, butyl, iso-butyl, sec-butyl, tert-butyl, pentyl, iso-pentyl, hexyl, heptyl, octyl, nonyl, decyl, undecyl, dodecyl, and optionally substituted phenyl.

In some embodiments, Compounds of the Disclosure are compounds of any one of Formulae (I)—(III), wherein R¹ and R² are isopropyl.

In some embodiments, Compounds of the Disclosure are compounds of any one of Formulae (I)—(III), wherein R⁴, R⁵, R⁶, R⁷, R⁸, R⁹, R¹⁰, and R¹¹ are selected from the group consisting of hydrogen and methyl.

In some embodiments, Compounds of the Disclosure are compounds of any one of Formulae (I)—(III), wherein R⁴, R⁵, R⁶, R⁷, R⁸, R⁹, R¹⁰, and R¹¹ are hydrogen.

In some embodiments, Compounds of the Disclosure are compounds of any one of Formulae (I)—(III), wherein X is —CHR^{3a}; R^{3a} is selected from the group consisting of C₁-C₁₂ alkyl,

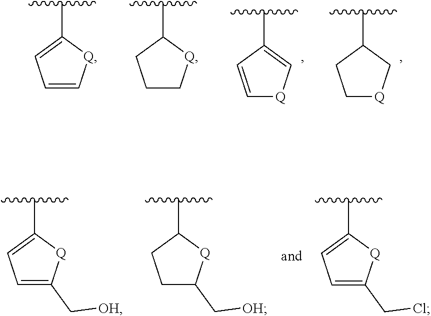

and Q is —NH—, —O—, or —S—.

In some embodiments, Compounds of the Disclosure are compounds of any one of Formulae (I)—(III), wherein X is —CHR^{3a} and R^{3a} is C₁-C₁₂ alkyl.

In some embodiments, Compounds of the Disclosure are compounds of any one of Formulae (I)—(III), wherein X is —CHR^{3a} and R^{3a} is methyl, ethyl, propyl, isopropyl, butyl, iso-butyl, sec-butyl, or tert-butyl.

In some embodiments, Compounds of the Disclosure are compounds of any one of Formulae (I)—(III), wherein X is —CHR^{3a} and R^{3a} is selected from the group consisting of C₂-C₁₂ alkyl, C₃-C₆ cycloalkyl, optionally substituted phenyl, optionally substituted 5- or 6-membered heterocyclo, and optionally substituted 5- or 6-membered heteroaryl.

In some embodiments, Compounds of the Disclosure are compounds of any one of Formulae (I)—(III), wherein X is —CHR^{3a} and R^{3a} is C₃-C₆ cycloalkyl.

In some embodiments, Compounds of the Disclosure are compounds of any one of Formulae (I)—(III), wherein X is —CHR^{3a} and R^{3a} is optionally substituted phenyl.

In some embodiments, Compounds of the Disclosure are compounds of any one of Formulae (I)—(III), wherein X is —CHR^{3a} and R^{3a} is optionally substituted 5- or 6-membered heterocyclo.

In some embodiments, Compounds of the Disclosure are compounds of any one of Formulae (I)—(III), wherein X is —CHR^{3a} and R^{3a} is optionally substituted 5- or 6-membered heteroaryl.

In some embodiments, Compounds of the Disclosure are compounds of any one of Formulae (I)—(III), wherein X is —CHR^{3a} and R^{3a} is:

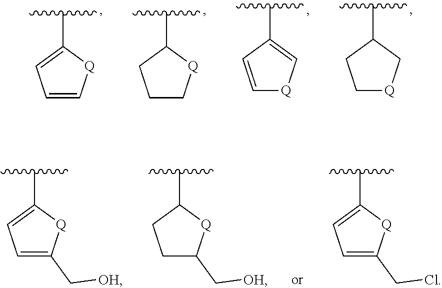

In some embodiments, Compounds of the Disclosure are compounds of any one of Formulae (I)—(III), wherein Q is —O—.

In another embodiment, Compounds of the Disclosure are compounds having Formula (IV):

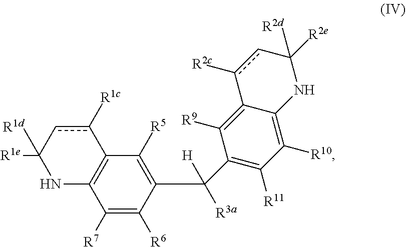

or a salt or solvate thereof, wherein:
each [symbol] is a single or double bond;
R^{1c}, R^{1d}, R^{1e}, R^{2c}, R^{2d}, and R^{2e} are independently selected from the group consisting of hydrogen, C₁-C₉ alkyl, C₃-C₆ cycloalkyl, and optionally substituted phenyl; and
R⁵, R⁶, R⁷, R⁹, R¹⁰, and R¹¹ are as defined in connection with Formula (I).

In some embodiments, Compounds of the Disclosure are compounds of Formula (IV), wherein R^{1c}, R^{1e}, R^{2c}, and R^{2e} are independently selected from the group consisting of hydrogen and C₂-C₆ alkyl. In some embodiments, R^{1c}, R^{1e}, R^{2c}, and R^{2e} are independently C₂-C₆ alkyl. In some embodiments, R^{1c}, R^{1e}, R^{2c}, and R^{2e} are ethyl.

In another embodiment, Compounds of the Disclosure are compounds having Formula (IVa):

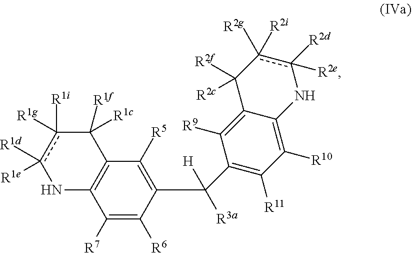

or a salt or solvate thereof, wherein:
each [symbol] is a single or double bond;
R^{1f} and R^{2f} are independently selected from the group consisting of hydrogen, halogen, C₁-C₉ alkyl, C₁-C₉ haloalkyl, C₃-C₈ cycloalkyl, —OH, —SH, C₁-C₈ alkoxy, C₁-C₈ alkylthio, arylthio, —C(═O)OR, —OC(═O)R, —C(═O)NRR′, —NRC(═O)R′, —NRR′, —SC(═O)R, —SC(═O)SR, —SC(═O)NRR′, —NRC(═O)SR′, —NRC(═S)SR′, 4- to 6-membered heterocyclyl, optionally substituted phenyl, and optionally substituted 5- or 6-membered heteroaryl;
R and R′ are at each occurrence independently selected from the group consisting of hydrogen, C₁-C₉ alkyl, C₃-C₆ cycloalkyl, and optionally substituted phenyl;
R^{1g}, R^{2g}, R¹ⁱ, and R²ⁱ are independently selected from the group consisting of hydrogen C₁-C₉ alkyl, C₃-C₆ cycloalkyl, and optionally substituted phenyl, with the proviso that if the [symbol] linking the carbons attached to R^{1e} and R¹ⁱ is a double bond, R^{1e} and R¹ⁱ are absent, and if the [symbol] linking the carbons attached to R^{2e} and R²ⁱ is a double bond, R^{2e} and R²ⁱ are absent;
R⁵, R⁶, R⁷, R⁹, R¹⁰, and R¹¹ are as defined in connection with Formula (I); and
R^{1c}, R^{1d}, R^{1e}, R^{2c}, R^{2d}, and R^{2e} are as defined in connection with Formula (IV).

In some embodiments, Compounds of the Disclosure are compounds of Formula (IVa), wherein [symbol] is a single bond.

In some embodiments, Compounds of the Disclosure are compounds of Formula (IVa), wherein [symbol] is a single bond and R^{1c}, R^{1f}, R^{2c}, and R^{2f} are methyl.

In some embodiments, Compounds of the Disclosure are compounds of Formula (IVa), wherein [symbol] is a single bond and R¹ⁱ, R^{1g}, R²ⁱ, and R^{2g} are methyl.

In another embodiment, Compounds of the Disclosure are compounds having Formula (V):

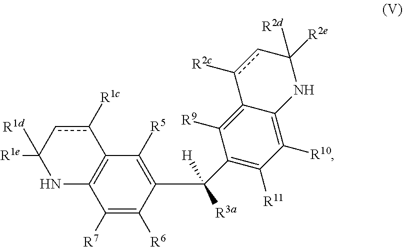

or a salt or solvate thereof, wherein:
each [symbol] is a single or double bond;
R^{1c}, R^{1d}, R^{1e}, R^{2c}, R^{2d}, and R^{2e} are independently selected from the group consisting of hydrogen and C₁-C₆ alkyl; and
R⁵, R⁶, R⁷, R⁹, R¹⁰, and R¹¹ are as defined in connection with Formula (I),
with the proviso that

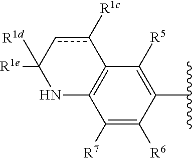

does not equal

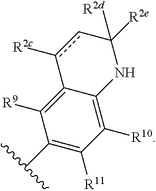

In another embodiment, Compounds of the Disclosure are compounds having Formula (Va):

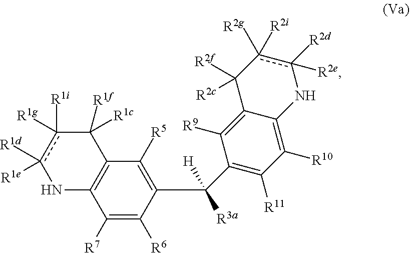

or a salt or solvate thereof, wherein:
each [symbol] is a single or double bond;
with the proviso that if the [symbol] linking the carbons attached to R^{1e} and R¹ⁱ is a double bond, R^{1e} and R¹ⁱ are absent, and if the [symbol] linking the carbons attached to R^{2e} and R²ⁱ is a double bond, R^{2e} and R²ⁱ are absent;
R⁵, R⁶, R⁷, R⁹, R¹⁰, and R¹¹ are as defined in connection with Formula (I);
R^{1c}, R^{1d}, R^{1e}, R^{2c}, R^{2d}, and R^{2e} are as defined in connection with Formula (IV); and
R^{1f}, R^{1g}, R¹ⁱ, R^{2f}, R^{2g}, and R²ⁱ are as defined in connection with Formula (IVa), with the proviso that

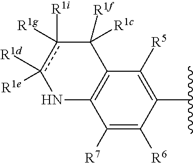

does not equal

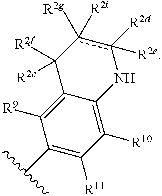

In some embodiments, Compounds of the Disclosure are compounds of Formula (Va), wherein [symbol] is a single bond.

In another embodiment, Compounds of the Disclosure are compounds having Formula (VI):

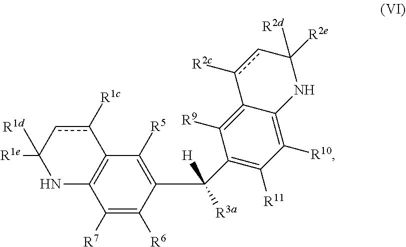

or a salt or solvate thereof, wherein:
each [symbol] a single or double bond;
R^{1c}, R^{1d}, R^{1e}, R^{2c}, R^{2d}, and R^{2e} are independently selected from the group consisting of hydrogen and C₁-C₆ alkyl; and
R⁵, R⁶, R⁷, R⁹, R¹⁰, and R¹¹ are as defined in connection with Formula (I), with the proviso that

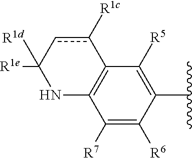

does not equal

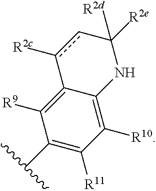

In another embodiment, Compounds of the Disclosure are compounds having Formula (VIa):

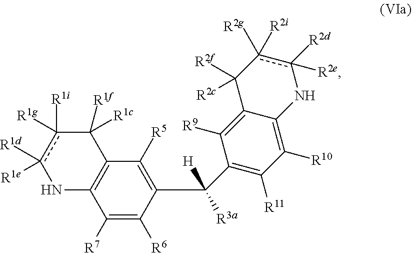

or a salt or solvate thereof, wherein:
each [symbol] is a single or double bond;
with the proviso that if the [symbol] linking the carbons attached to R^{1e} and R¹ⁱ is a double bond, R^{1e} and R¹ⁱ are absent, and if the [symbol] linking the carbons attached to R^{2e} and R²ⁱ is a double bond, R^{2e} and R²ⁱ are absent;
R⁵, R⁶, R⁷, R⁹, R¹⁰, and R¹¹ are as defined in connection with Formula (I);
R^{1c}, R^{1d}, R^{1e}, R^{2c}, R^{2d}, and R^{2e} are as defined in connection with Formula (IV); and
R^{1f}, R^{1g}, R¹ⁱ, R^{2f}, R^{2g}, and R²ⁱ are as defined in connection with Formula (IVa), with the proviso that

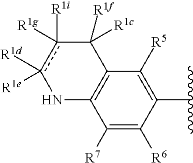

does not equal

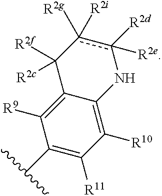

In some embodiments, Compounds of the Disclosure are compounds of Formula (VIa), wherein [symbol] is a single bond.

In some embodiments, Compounds of the Disclosure are compounds of any one of Formulae (IV)—(VI), (IVa), (Va), or (VIa), wherein R⁵, R⁶, R⁷, R⁹, R¹⁰, and R¹¹ are selected from the group consisting of hydrogen, methyl, C₁-C₆ alkoxy, and C₁-C₆ alkylthio.

In some embodiments, Compounds of the Disclosure are compounds of any one of Formulae (IV)—(VI), (IVa), (Va), or (VIa), wherein R⁵, R⁶, R⁷, R⁹, R¹⁰, and R¹¹ are hydrogen.

In some embodiments, Compounds of the Disclosure are compounds of any one of Formulae (IV)—(VI), (IVa), (Va), or (VIa), wherein R^{1c}, R^{1d}, R^{1e}, R^{2c}, R^{2d}, and R^{2e} are methyl, ethyl, or hydrogen.

In some embodiments, Compounds of the Disclosure are compounds of any one of Formulae (IV)—(VI), (IVa), (Va), or (VIa), wherein R^{1c}, R^{1d}, R^{1e}, R^{2c}, R^{2d}, and R^{2e} are methyl.

In some embodiments, Compounds of the Disclosure are compounds of any one of Formulae (IV)—(VI), (IVa), (Va), or (VIa), wherein R^{3a} is hydrogen.

In some embodiments, Compounds of the Disclosure are compounds of any one of Formulae (IV)—(VI), (IVa), (Va), or (VIa), wherein R^{3a} is selected from the group consisting of C₁-C₁₂ alkyl,

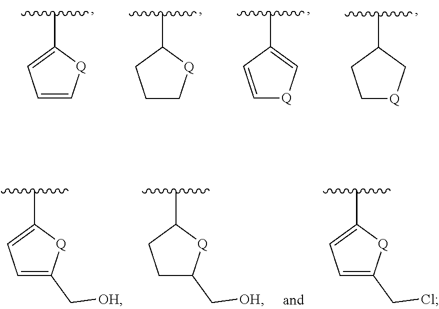

and Q is —NH—, —O—, or —S—.

In some embodiments, Compounds of the Disclosure are compounds of any one of Formulae (IV)—(VI), (IVa), (Va), or (VIa), wherein R^{3a} is C₁-C₁₂ alkyl.

In some embodiments, Compounds of the Disclosure are compounds of any one of Formulae (IV)—(VI), (IVa), (Va), or (VIa), wherein R^{3a} is methyl, ethyl, propyl, isopropyl, butyl, iso-butyl, sec-butyl, or tert-butyl.

In some embodiments, Compounds of the Disclosure are compounds of any one of Formulae (IV)—(VI), (IVa), (Va), or (VIa), wherein R^{3a} is:

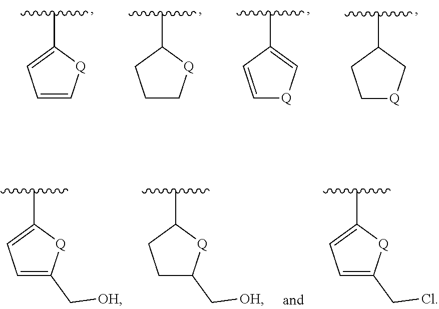

In some embodiments, Compounds of the Disclosure are compounds of any one of Formulae (IV)—(VI), (IVa), (Va), or (VIa), wherein Q is —O—.

In some embodiments, Compounds of the Disclosure are any one or more of the compounds of Table 1, or a salt or solvate thereof.

TABLE 1

| Compound | |
|---|---|
| No. | Structure |
| 1 | 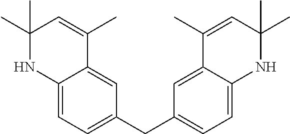 |
| 2 | 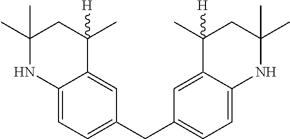 |
| 3 | 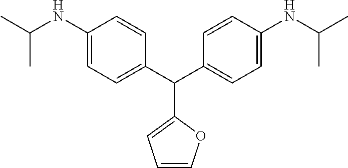 [SMILES: CC(C)Nc1ccc(C(c2ccc(NC(C)C)cc2)c2ccco2)cc1] |
| 4 | 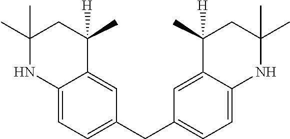 |
| 5 | 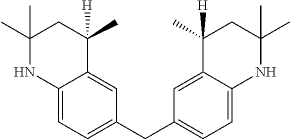 |
| 6 | 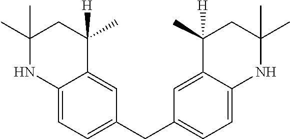 |
| 7 | 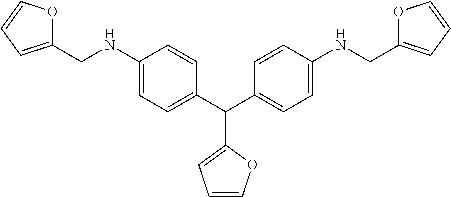 [SMILES: c1coc(CNc2ccc(C(c3ccc(NCc4ccco4)cc3)c3ccco3)cc2)c1] |
| 8 | 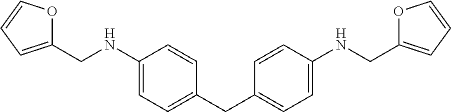 [SMILES: c1coc(CNc2ccc(Cc3ccc(NCc4ccco4)cc3)cc2)c1] |
| 9 | 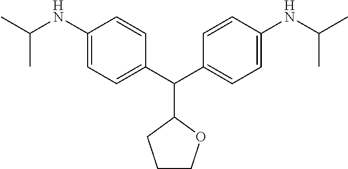 [SMILES: CC(C)Nc1ccc(C(c2ccc(NC(C)C)cc2)C2CCCO2)cc1] |
| 10 | 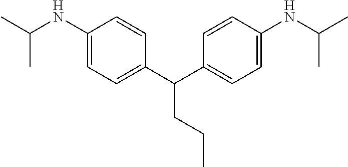 [SMILES: CCCC(c1ccc(NC(C)C)cc1)c1ccc(NC(C)C)cc1] |
| 11 | 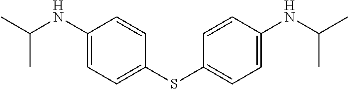 [SMILES: CC(C)Nc1ccc(Sc2ccc(NC(C)C)cc2)cc1] |
| 12 | 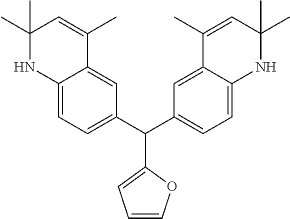 |
| 13 | 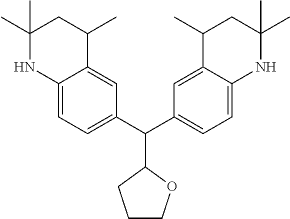 |
| 14 | 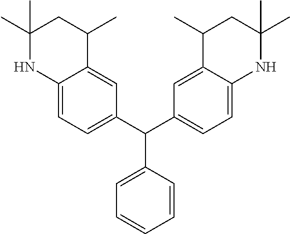 |
| 15 | 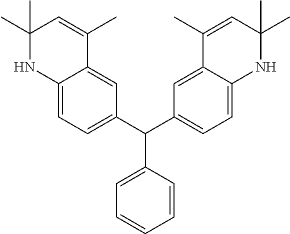 |
| 16 | 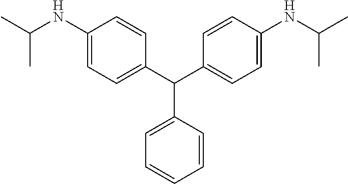 [SMILES: CC(C)Nc1ccc(C(c2ccccc2)c2ccc(NC(C)C)cc2)cc1] |
| 17 | 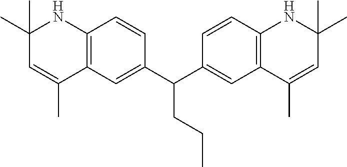 |
| 18 | 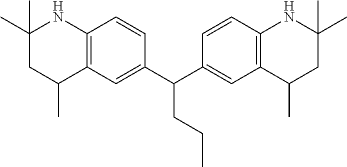 |
| 19 | 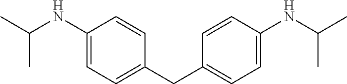 [SMILES: CC(C)Nc1ccc(Cc2ccc(NC(C)C)cc2)cc1] |
| 20 | 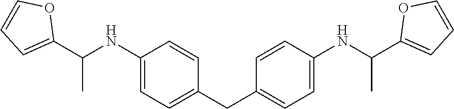 [SMILES: CC(Nc1ccc(Cc2ccc(NC(C)c3ccco3)cc2)cc1)c1ccco1] |
| 21 | 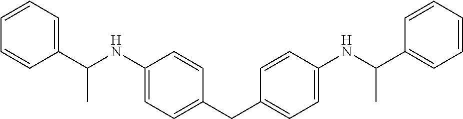 [SMILES: CC(Nc1ccc(Cc2ccc(NC(C)c3ccccc3)cc2)cc1)c1ccccc1] |
| 22 | 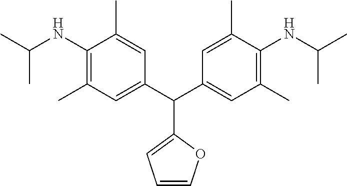 [SMILES: Cc1cc(C(c2cc(C)c(NC(C)C)c(C)c2)c2ccco2)cc(C)c1NC(C)C] |
| 23 | 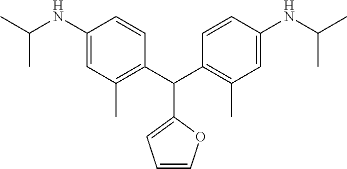 [SMILES: Cc1cc(NC(C)C)ccc1C(c1ccco1)c1ccc(NC(C)C)cc1C] |
| 24 | 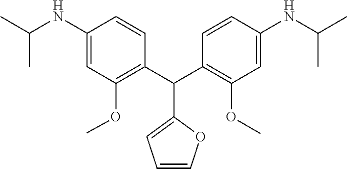 [SMILES: COc1cc(NC(C)C)ccc1C(c1ccco1)c1ccc(NC(C)C)cc1OC] |
| 25 | 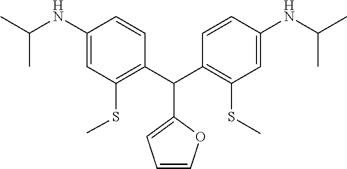 [SMILES: CSc1cc(NC(C)C)ccc1C(c1ccco1)c1ccc(NC(C)C)cc1SC] |
| 26 | 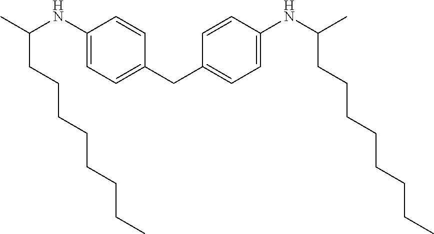 [SMILES: CCCCCCCCC(C)Nc1ccc(Cc2ccc(NC(C)CCCCCCCC)cc2)cc1] |
| 27 | 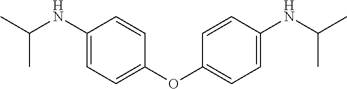 [SMILES: CC(C)Nc1ccc(Oc2ccc(NC(C)C)cc2)cc1] |
| 28 | 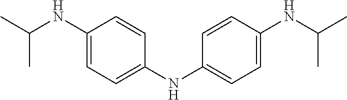 [SMILES: CC(C)Nc1ccc(Nc2ccc(NC(C)C)cc2)cc1] |
| 29 | 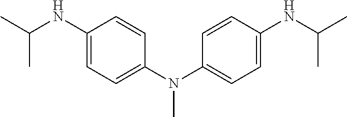 [SMILES: CC(C)Nc1ccc(N(C)c2ccc(NC(C)C)cc2)cc1] |
| 30 | 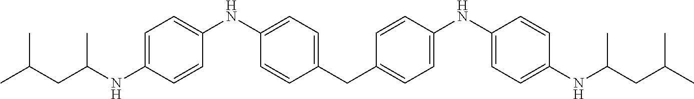 [SMILES: CC(C)CC(C)Nc1ccc(Nc2ccc(Cc3ccc(Nc4ccc(NC(C)CC(C)C)cc4)cc3)cc2)cc1] |
| 31 | 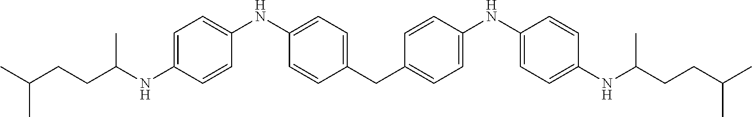 [SMILES: CC(C)CCC(C)Nc1ccc(Nc2ccc(Cc3ccc(Nc4ccc(NC(C)CCC(C)C)cc4)cc3)cc2)cc1] |
| 32 | 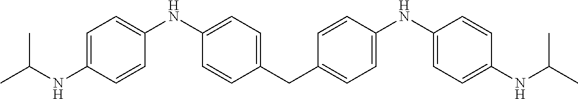 [SMILES: CC(C)Nc1ccc(Nc2ccc(Cc3ccc(Nc4ccc(NC(C)C)cc4)cc3)cc2)cc1] |
| 33 | 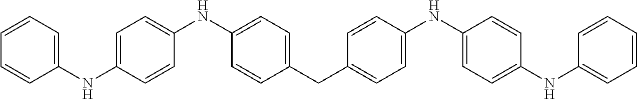 [SMILES: c1ccc(Nc2ccc(Nc3ccc(Cc4ccc(Nc5ccc(Nc6ccccc6)cc5)cc4)cc3)cc2)cc1] |
| 34 | 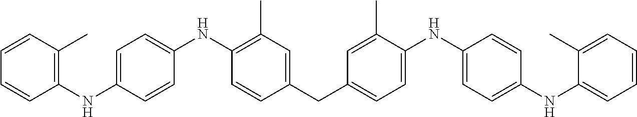 [SMILES: Cc1ccccc1Nc1ccc(Nc2ccc(Cc3ccc(Nc4ccc(Nc5ccccc5C)cc4)c(C)c3)cc2C)cc1] |
| 35 | 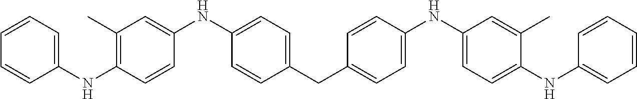 [SMILES: Cc1cc(Nc2ccc(Cc3ccc(Nc4ccc(Nc5ccccc5)c(C)c4)cc3)cc2)ccc1Nc1ccccc1] |
| 36 | 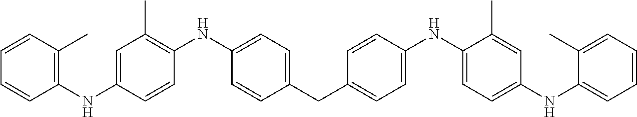 [SMILES: Cc1ccccc1Nc1ccc(Nc2ccc(Cc3ccc(Nc4ccc(Nc5ccccc5C)cc4C)cc3)cc2)c(C)c1] |
| 37 | 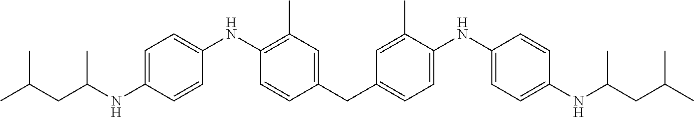 [SMILES: Cc1cc(Cc2ccc(Nc3ccc(NC(C)CC(C)C)cc3)c(C)c2)ccc1Nc1ccc(NC(C)CC(C)C)cc1] |
| 38 | 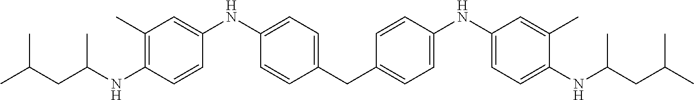 [SMILES: Cc1cc(Nc2ccc(Cc3ccc(Nc4ccc(NC(C)CC(C)C)c(C)c4)cc3)cc2)ccc1NC(C)CC(C)C] |
| 39 | 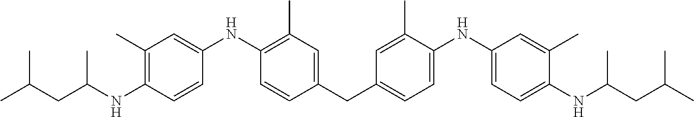 [SMILES: Cc1cc(Cc2ccc(Nc3ccc(NC(C)CC(C)C)c(C)c3)c(C)c2)ccc1Nc1ccc(NC(C)CC(C)C)c(C)c1] |
| 40 | 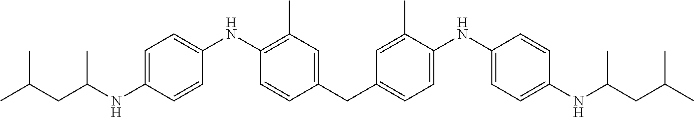 [SMILES: Cc1cc(Cc2ccc(Nc3ccc(NC(C)CC(C)C)cc3)c(C)c2)ccc1Nc1ccc(NC(C)CC(C)C)cc1] |
| 41 | 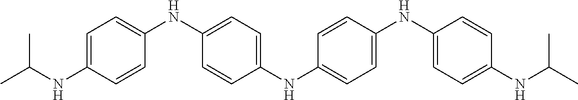 [SMILES: CC(C)Nc1ccc(Nc2ccc(Nc3ccc(Nc4ccc(NC(C)C)cc4)cc3)cc2)cc1] |
| 42 | 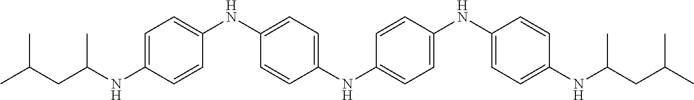 [SMILES: CC(C)CC(C)Nc1ccc(Nc2ccc(Nc3ccc(Nc4ccc(NC(C)CC(C)C)cc4)cc3)cc2)cc1] |
| 43 | 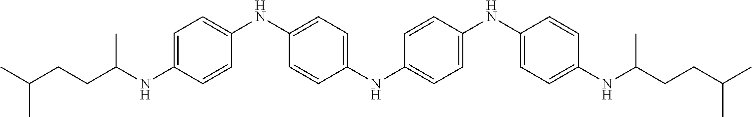 [SMILES: CC(C)CCC(C)Nc1ccc(Nc2ccc(Nc3ccc(Nc4ccc(NC(C)CCC(C)C)cc4)cc3)cc2)cc1] |
| 44 | 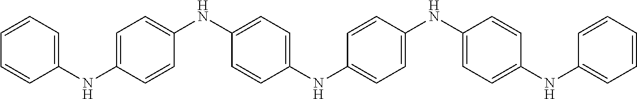 [SMILES: c1ccc(Nc2ccc(Nc3ccc(Nc4ccc(Nc5ccc(Nc6ccccc6)cc5)cc4)cc3)cc2)cc1] |
| 45 | 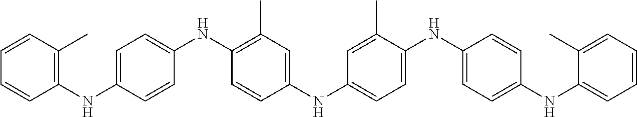 [SMILES: Cc1ccccc1Nc1ccc(Nc2ccc(Nc3ccc(Nc4ccc(Nc5ccccc5C)cc4)c(C)c3)cc2C)cc1] |
| 46 | 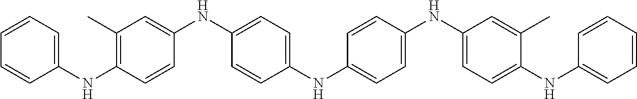 [SMILES: Cc1cc(Nc2ccc(Nc3ccc(Nc4ccc(Nc5ccccc5)c(C)c4)cc3)cc2)ccc1Nc1ccccc1] |
| 47 | 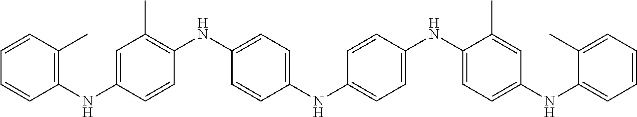 [SMILES: Cc1ccccc1Nc1ccc(Nc2ccc(Nc3ccc(Nc4ccc(Nc5ccccc5C)cc4C)cc3)cc2)c(C)c1] |
| 48 | 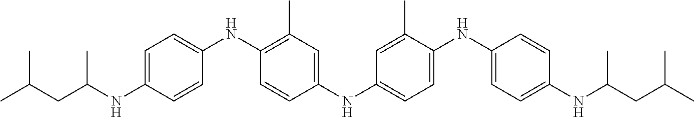 [SMILES: Cc1cc(Nc2ccc(Nc3ccc(NC(C)CC(C)C)cc3)c(C)c2)ccc1Nc1ccc(NC(C)CC(C)C)cc1] |
| 49 | 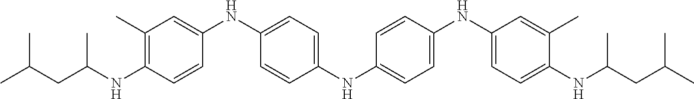 [SMILES: Cc1cc(Nc2ccc(Nc3ccc(Nc4ccc(NC(C)CC(C)C)c(C)c4)cc3)cc2)ccc1NC(C)CC(C)C] |
| 50 | 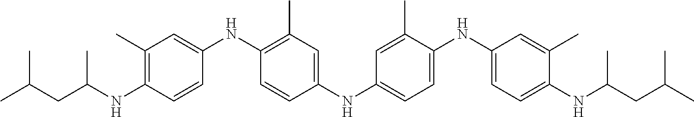 [SMILES: Cc1cc(Nc2ccc(Nc3ccc(NC(C)CC(C)C)c(C)c3)c(C)c2)ccc1Nc1ccc(NC(C)CC(C)C)c(C)c1] |
| 51 | 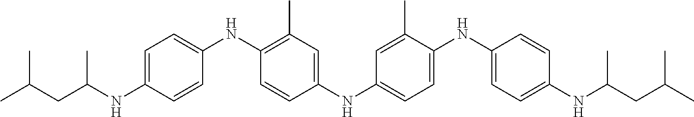 [SMILES: Cc1cc(Nc2ccc(Nc3ccc(NC(C)CC(C)C)cc3)c(C)c2)ccc1Nc1ccc(NC(C)CC(C)C)cc1] |
| 52 | 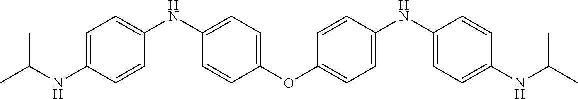 [SMILES: CC(C)Nc1ccc(Nc2ccc(Oc3ccc(Nc4ccc(NC(C)C)cc4)cc3)cc2)cc1] |
| 53 | 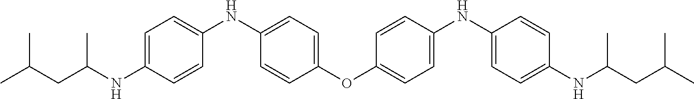 [SMILES: CC(C)CC(C)Nc1ccc(Nc2ccc(Oc3ccc(Nc4ccc(NC(C)CC(C)C)cc4)cc3)cc2)cc1] |
| 54 | 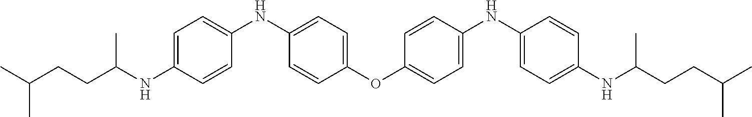 [SMILES: CC(C)CCC(C)Nc1ccc(Nc2ccc(Oc3ccc(Nc4ccc(NC(C)CCC(C)C)cc4)cc3)cc2)cc1] |
| 55 | 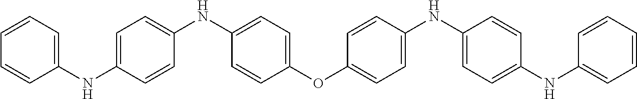 [SMILES: c1ccc(Nc2ccc(Nc3ccc(Oc4ccc(Nc5ccc(Nc6ccccc6)cc5)cc4)cc3)cc2)cc1] |
| 56 | 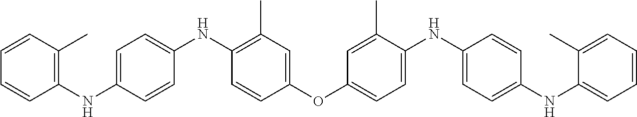 [SMILES: Cc1ccccc1Nc1ccc(Nc2ccc(Oc3ccc(Nc4ccc(Nc5ccccc5C)cc4)c(C)c3)cc2C)cc1] |
| 57 | 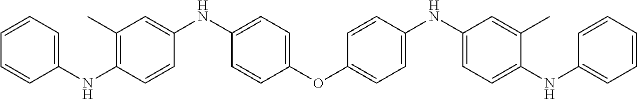 [SMILES: Cc1cc(Nc2ccc(Oc3ccc(Nc4ccc(Nc5ccccc5)c(C)c4)cc3)cc2)ccc1Nc1ccccc1] |
| 58 | 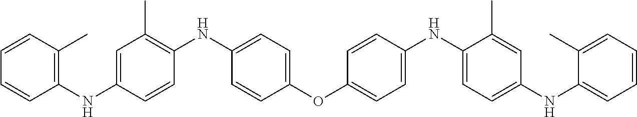 [SMILES: Cc1ccccc1Nc1ccc(Nc2ccc(Oc3ccc(Nc4ccc(Nc5ccccc5C)cc4C)cc3)cc2)c(C)c1] |
| 59 | 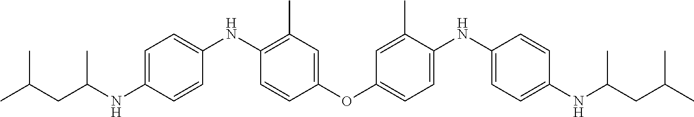 [SMILES: Cc1cc(Oc2ccc(Nc3ccc(NC(C)CC(C)C)cc3)c(C)c2)ccc1Nc1ccc(NC(C)CC(C)C)cc1] |
| 60 | 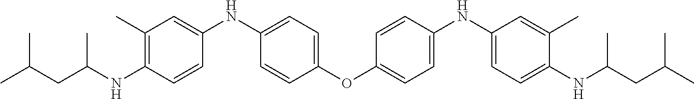 [SMILES: Cc1cc(Nc2ccc(Oc3ccc(Nc4ccc(NC(C)CC(C)C)c(C)c4)cc3)cc2)ccc1NC(C)CC(C)C] |
| 61 | 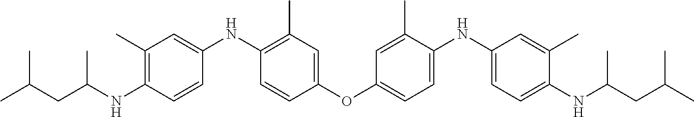 [SMILES: Cc1cc(Oc2ccc(Nc3ccc(NC(C)CC(C)C)c(C)c3)c(C)c2)ccc1Nc1ccc(NC(C)CC(C)C)c(C)c1] |
| 62 | 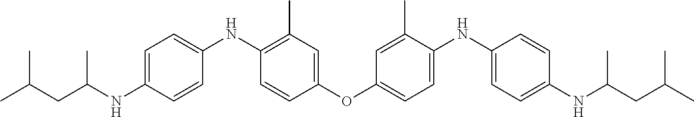 [SMILES: Cc1cc(Oc2ccc(Nc3ccc(NC(C)CC(C)C)cc3)c(C)c2)ccc1Nc1ccc(NC(C)CC(C)C)cc1] |
| 63 | 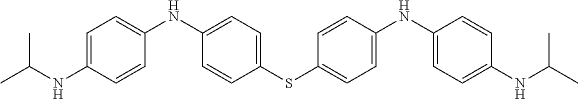 [SMILES: CC(C)Nc1ccc(Nc2ccc(Sc3ccc(Nc4ccc(NC(C)C)cc4)cc3)cc2)cc1] |
| 64 | 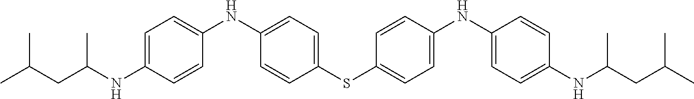 [SMILES: CC(C)CC(C)Nc1ccc(Nc2ccc(Sc3ccc(Nc4ccc(NC(C)CC(C)C)cc4)cc3)cc2)cc1] |
| 65 | 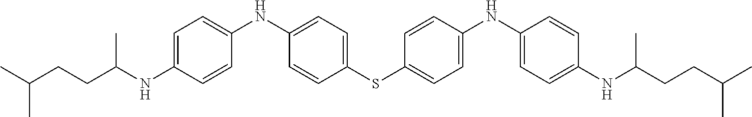 [SMILES: CC(C)CCC(C)Nc1ccc(Nc2ccc(Sc3ccc(Nc4ccc(NC(C)CCC(C)C)cc4)cc3)cc2)cc1] |
| 66 | 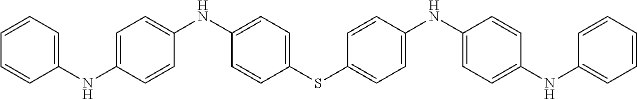 [SMILES: c1ccc(Nc2ccc(Nc3ccc(Sc4ccc(Nc5ccc(Nc6ccccc6)cc5)cc4)cc3)cc2)cc1] |
| 67 | 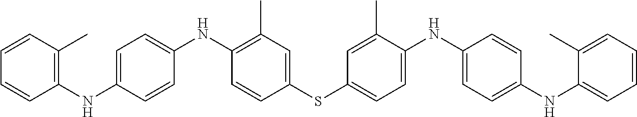 [SMILES: Cc1ccccc1Nc1ccc(Nc2ccc(Sc3ccc(Nc4ccc(Nc5ccccc5C)cc4)c(C)c3)cc2C)cc1] |
| 68 | 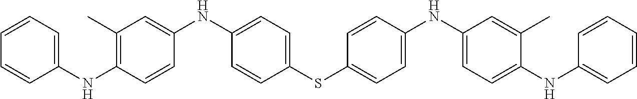 [SMILES: Cc1cc(Nc2ccc(Sc3ccc(Nc4ccc(Nc5ccccc5)c(C)c4)cc3)cc2)ccc1Nc1ccccc1] |
| 69 | 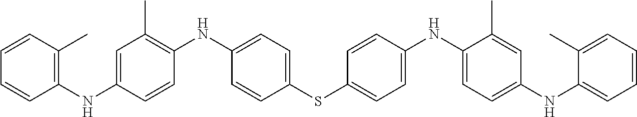 [SMILES: Cc1ccccc1Nc1ccc(Nc2ccc(Sc3ccc(Nc4ccc(Nc5ccccc5C)cc4C)cc3)cc2)c(C)c1] |
| 70 | 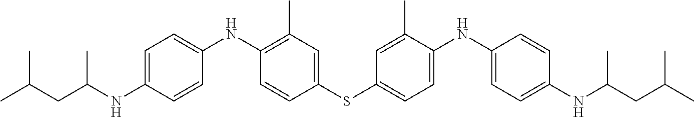 [SMILES: Cc1cc(Sc2ccc(Nc3ccc(NC(C)CC(C)C)cc3)c(C)c2)ccc1Nc1ccc(NC(C)CC(C)C)cc1] |
| 71 | 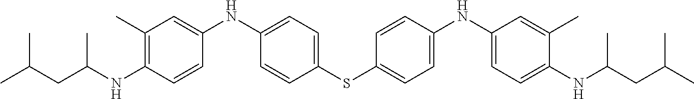 [SMILES: Cc1cc(Nc2ccc(Sc3ccc(Nc4ccc(NC(C)CC(C)C)c(C)c4)cc3)cc2)ccc1NC(C)CC(C)C] |
| 72 | 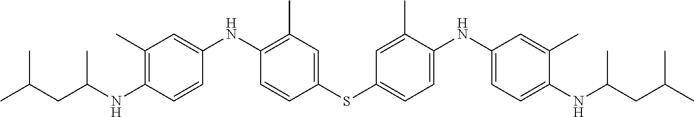 [SMILES: Cc1cc(Sc2ccc(Nc3ccc(NC(C)CC(C)C)c(C)c3)c(C)c2)ccc1Nc1ccc(NC(C)CC(C)C)c(C)c1] |
| 73 | 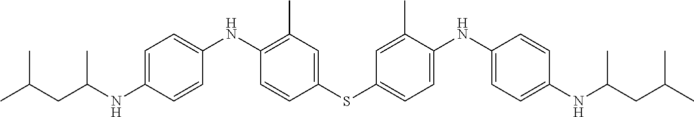 [SMILES: Cc1cc(Sc2ccc(Nc3ccc(NC(C)CC(C)C)cc3)c(C)c2)ccc1Nc1ccc(NC(C)CC(C)C)cc1] |
| 74 | 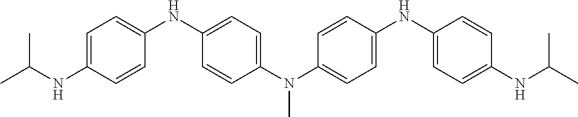 [SMILES: CC(C)Nc1ccc(Nc2ccc(N(C)c3ccc(Nc4ccc(NC(C)C)cc4)cc3)cc2)cc1] |
| 75 | 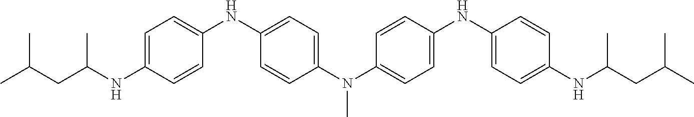 [SMILES: CC(C)CC(C)Nc1ccc(Nc2ccc(N(C)c3ccc(Nc4ccc(NC(C)CC(C)C)cc4)cc3)cc2)cc1] |
| 76 | 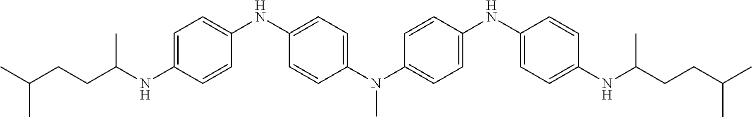 [SMILES: CC(C)CCC(C)Nc1ccc(Nc2ccc(N(C)c3ccc(Nc4ccc(NC(C)CCC(C)C)cc4)cc3)cc2)cc1] |
| 77 | 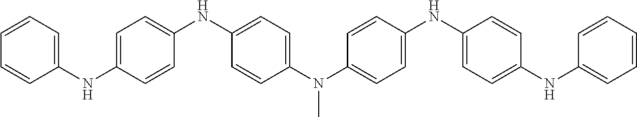 [SMILES: CN(c1ccc(Nc2ccc(Nc3ccccc3)cc2)cc1)c1ccc(Nc2ccc(Nc3ccccc3)cc2)cc1] |
| 78 | 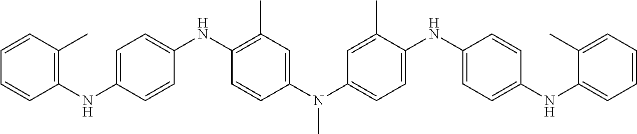 [SMILES: Cc1ccccc1Nc1ccc(Nc2ccc(N(C)c3ccc(Nc4ccc(Nc5ccccc5C)cc4)c(C)c3)cc2C)cc1] |
| 79 | 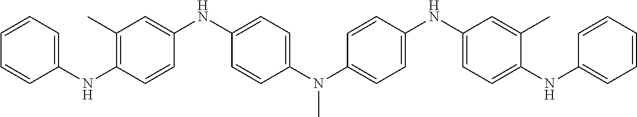 [SMILES: Cc1cc(Nc2ccc(N(C)c3ccc(Nc4ccc(Nc5ccccc5)c(C)c4)cc3)cc2)ccc1Nc1ccccc1] |
| 80 | 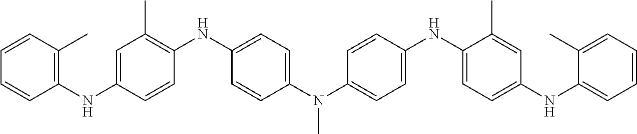 [SMILES: Cc1ccccc1Nc1ccc(Nc2ccc(N(C)c3ccc(Nc4ccc(Nc5ccccc5C)cc4C)cc3)cc2)c(C)c1] |
| 81 | 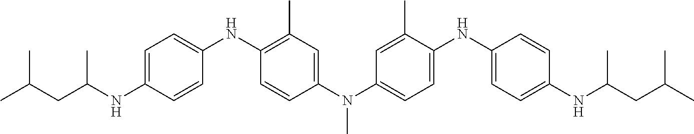 [SMILES: Cc1cc(N(C)c2ccc(Nc3ccc(NC(C)CC(C)C)cc3)c(C)c2)ccc1Nc1ccc(NC(C)CC(C)C)cc1] |
| 82 | 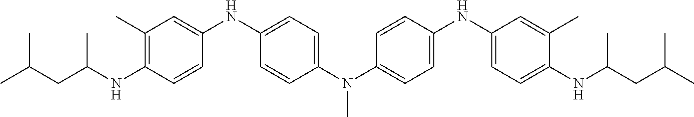 [SMILES: Cc1cc(Nc2ccc(N(C)c3ccc(Nc4ccc(NC(C)CC(C)C)c(C)c4)cc3)cc2)ccc1NC(C)CC(C)C] |
| 83 | 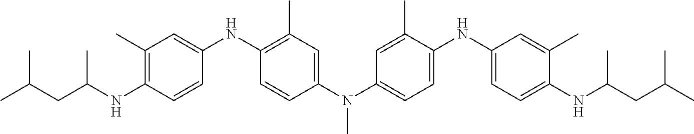 [SMILES: Cc1cc(N(C)c2ccc(Nc3ccc(NC(C)CC(C)C)c(C)c3)c(C)c2)ccc1Nc1ccc(NC(C)CC(C)C)c(C)c1] |
| 84 | 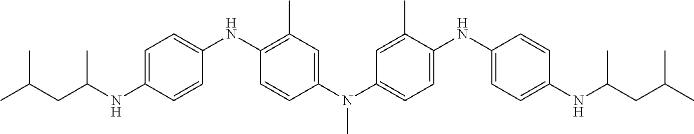 [SMILES: Cc1cc(N(C)c2ccc(Nc3ccc(NC(C)CC(C)C)cc3)c(C)c2)ccc1Nc1ccc(NC(C)CC(C)C)cc1] |
| 85 | 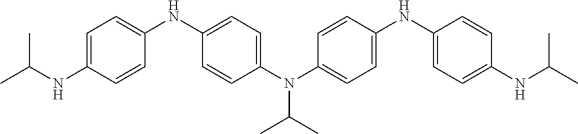 [SMILES: CC(C)Nc1ccc(Nc2ccc(N(c3ccc(Nc4ccc(NC(C)C)cc4)cc3)C(C)C)cc2)cc1] |
| 86 | 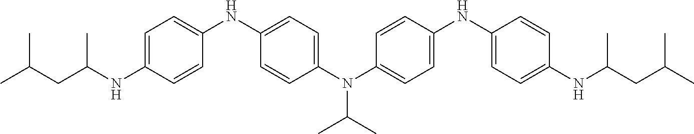 [SMILES: CC(C)CC(C)Nc1ccc(Nc2ccc(N(c3ccc(Nc4ccc(NC(C)CC(C)C)cc4)cc3)C(C)C)cc2)cc1] |
| 87 | 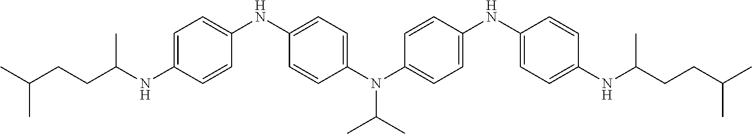 [SMILES: CC(C)CCC(C)Nc1ccc(Nc2ccc(N(c3ccc(Nc4ccc(NC(C)CCC(C)C)cc4)cc3)C(C)C)cc2)cc1] |
| 88 | 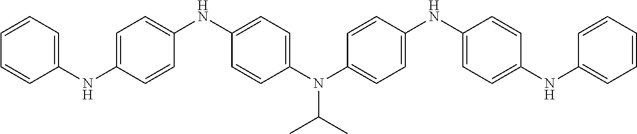 [SMILES: CC(C)N(c1ccc(Nc2ccc(Nc3ccccc3)cc2)cc1)c1ccc(Nc2ccc(Nc3ccccc3)cc2)cc1] |
| 89 | 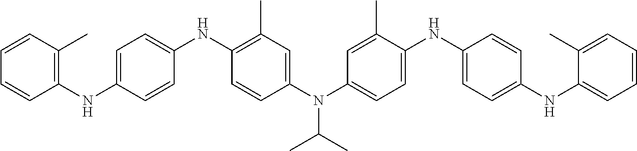 [SMILES: Cc1ccccc1Nc1ccc(Nc2ccc(N(c3ccc(Nc4ccc(Nc5ccccc5C)cc4)c(C)c3)C(C)C)cc2C)cc1] |
| 90 | 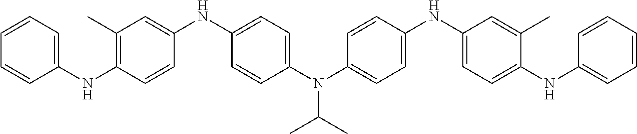 [SMILES: Cc1cc(Nc2ccc(N(c3ccc(Nc4ccc(Nc5ccccc5)c(C)c4)cc3)C(C)C)cc2)ccc1Nc1ccccc1] |
| 91 | 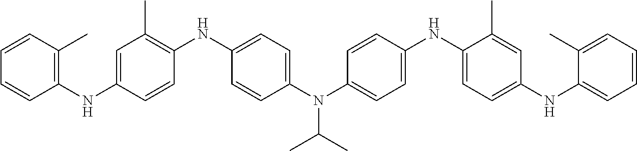 [SMILES: Cc1ccccc1Nc1ccc(Nc2ccc(N(c3ccc(Nc4ccc(Nc5ccccc5C)cc4C)cc3)C(C)C)cc2)c(C)c1] |
| 92 | 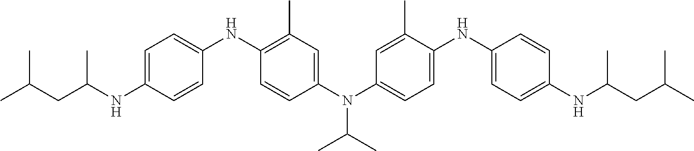 [SMILES: Cc1cc(N(c2ccc(Nc3ccc(NC(C)CC(C)C)cc3)c(C)c2)C(C)C)ccc1Nc1ccc(NC(C)CC(C)C)cc1] |
| 93 | 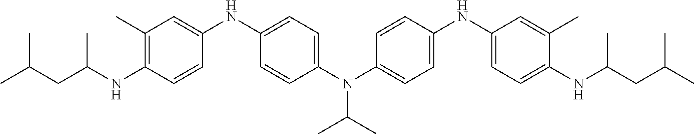 [SMILES: Cc1cc(Nc2ccc(N(c3ccc(Nc4ccc(NC(C)CC(C)C)c(C)c4)cc3)C(C)C)cc2)ccc1NC(C)CC(C)C] |
| 94 | 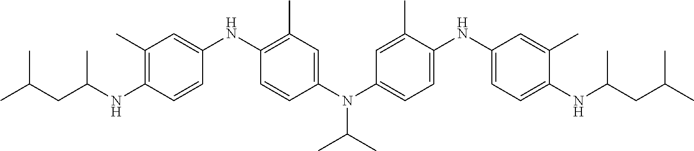 [SMILES: Cc1cc(N(c2ccc(Nc3ccc(NC(C)CC(C)C)c(C)c3)c(C)c2)C(C)C)ccc1Nc1ccc(NC(C)CC(C)C)c(C)c1] |
| 95 | 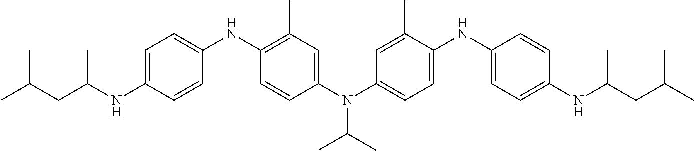 [SMILES: Cc1cc(N(c2ccc(Nc3ccc(NC(C)CC(C)C)cc3)c(C)c2)C(C)C)ccc1Nc1ccc(NC(C)CC(C)C)cc1] |
| 96 | 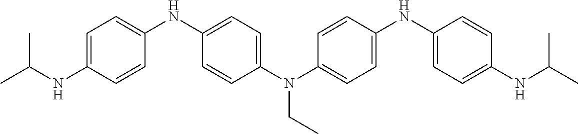 [SMILES: CCN(c1ccc(Nc2ccc(NC(C)C)cc2)cc1)c1ccc(Nc2ccc(NC(C)C)cc2)cc1] |
| 97 | 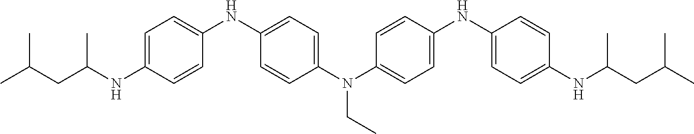 [SMILES: CCN(c1ccc(Nc2ccc(NC(C)CC(C)C)cc2)cc1)c1ccc(Nc2ccc(NC(C)CC(C)C)cc2)cc1] |
| 98 | 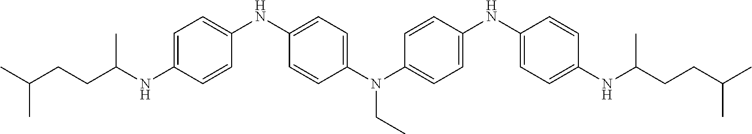 [SMILES: CCN(c1ccc(Nc2ccc(NC(C)CCC(C)C)cc2)cc1)c1ccc(Nc2ccc(NC(C)CCC(C)C)cc2)cc1] |
| 99 | 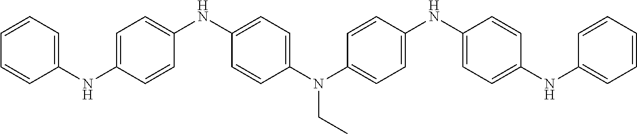 [SMILES: CCN(c1ccc(Nc2ccc(Nc3ccccc3)cc2)cc1)c1ccc(Nc2ccc(Nc3ccccc3)cc2)cc1] |
| 100 | 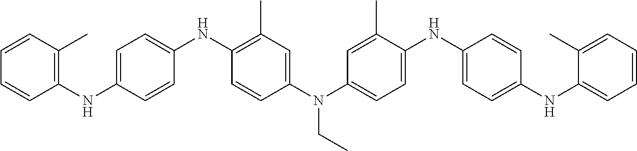 [SMILES: CCN(c1ccc(Nc2ccc(Nc3ccccc3C)cc2)c(C)c1)c1ccc(Nc2ccc(Nc3ccccc3C)cc2)c(C)c1] |
| 101 | 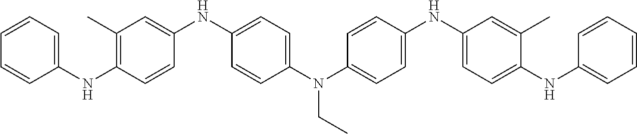 [SMILES: CCN(c1ccc(Nc2ccc(Nc3ccccc3)c(C)c2)cc1)c1ccc(Nc2ccc(Nc3ccccc3)c(C)c2)cc1] |
| 102 | 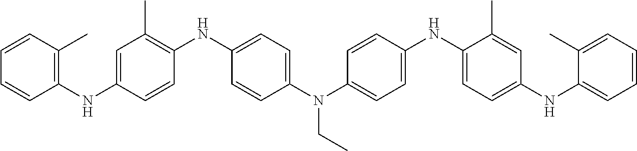 [SMILES: CCN(c1ccc(Nc2ccc(Nc3ccccc3C)cc2C)cc1)c1ccc(Nc2ccc(Nc3ccccc3C)cc2C)cc1] |
| 103 | 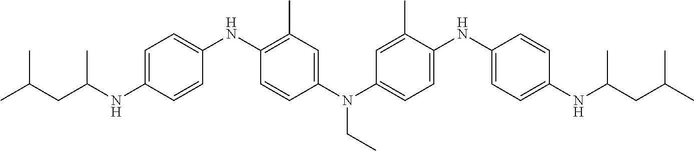 [SMILES: CCN(c1ccc(Nc2ccc(NC(C)CC(C)C)cc2)c(C)c1)c1ccc(Nc2ccc(NC(C)CC(C)C)cc2)c(C)c1] |
| 104 | 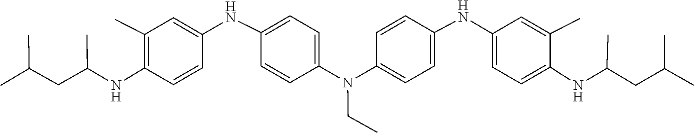 [SMILES: CCN(c1ccc(Nc2ccc(NC(C)CC(C)C)c(C)c2)cc1)c1ccc(Nc2ccc(NC(C)CC(C)C)c(C)c2)cc1] |
| 105 | 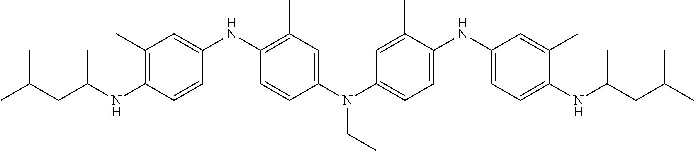 [SMILES: CCN(c1ccc(Nc2ccc(NC(C)CC(C)C)c(C)c2)c(C)c1)c1ccc(Nc2ccc(NC(C)CC(C)C)c(C)c2)c(C)c1] |
| 106 | 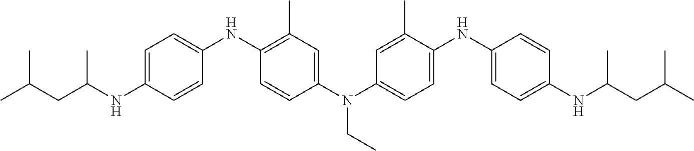 [SMILES: CCN(c1ccc(Nc2ccc(NC(C)CC(C)C)cc2)c(C)c1)c1ccc(Nc2ccc(NC(C)CC(C)C)cc2)c(C)c1] |
| 107 | 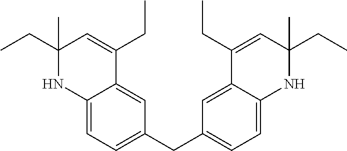 [SMILES: CCC1=CC(C)(CC)Nc2ccc(Cc3ccc4c(c3)C(CC)=CC(C)(CC)N4)cc21] |
| 108 | 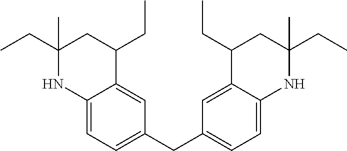 [SMILES: CCC1CC(C)(CC)Nc2ccc(Cc3ccc4c(c3)C(CC)CC(C)(CC)N4)cc21] |
| 109 | 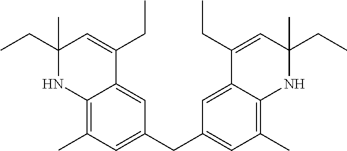 [SMILES: CCC1=CC(C)(CC)Nc2c(C)cc(Cc3cc(C)c4c(c3)C(CC)=CC(C)(CC)N4)cc21] |
| 110 | 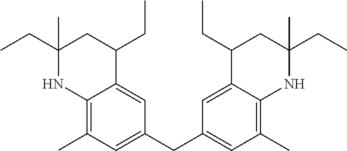 [SMILES: CCC1CC(C)(CC)Nc2c(C)cc(Cc3cc(C)c4c(c3)C(CC)CC(C)(CC)N4)cc21] |
| 111 | 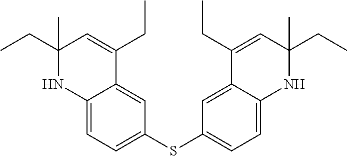 [SMILES: CCC1=CC(C)(CC)Nc2ccc(Sc3ccc4c(c3)C(CC)=CC(C)(CC)N4)cc21] |
| 112 | 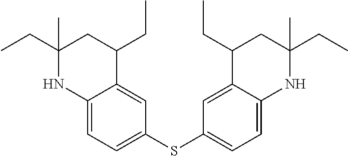 [SMILES: CCC1CC(C)(CC)Nc2ccc(Sc3ccc4c(c3)C(CC)CC(C)(CC)N4)cc21] |
| 113 | 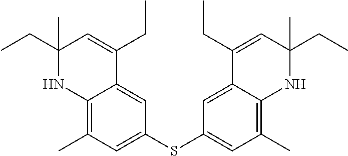 [SMILES: CCC1=CC(C)(CC)Nc2c(C)cc(Sc3cc(C)c4c(c3)C(CC)=CC(C)(CC)N4)cc21] |
| 114 | 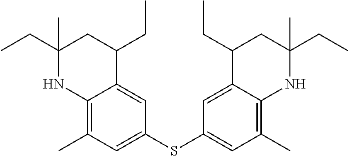 [SMILES: CCC1CC(C)(CC)Nc2c(C)cc(Sc3cc(C)c4c(c3)C(CC)CC(C)(CC)N4)cc21] |
| 115 | 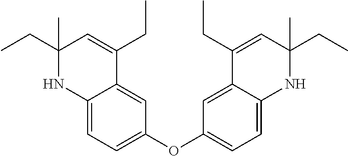 [SMILES: CCC1=CC(C)(CC)Nc2ccc(Oc3ccc4c(c3)C(CC)=CC(C)(CC)N4)cc21] |
| 116 | 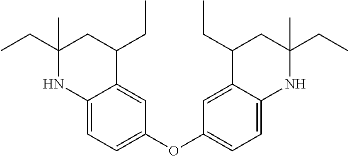 [SMILES: CCC1CC(C)(CC)Nc2ccc(Oc3ccc4c(c3)C(CC)CC(C)(CC)N4)cc21] |
| 117 | 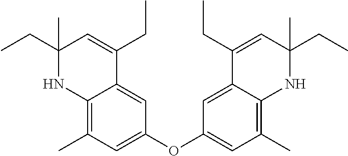 [SMILES: CCC1=CC(C)(CC)Nc2c(C)cc(Oc3cc(C)c4c(c3)C(CC)=CC(C)(CC)N4)cc21] |
| 118 | 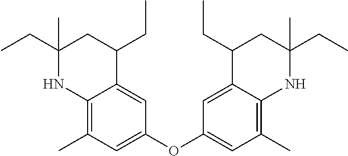 [SMILES: CCC1CC(C)(CC)Nc2c(C)cc(Oc3cc(C)c4c(c3)C(CC)CC(C)(CC)N4)cc21] |
| 119 | 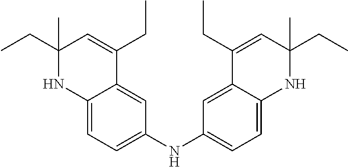 [SMILES: CCC1=CC(C)(CC)Nc2ccc(Nc3ccc4c(c3)C(CC)=CC(C)(CC)N4)cc21] |
| 120 | 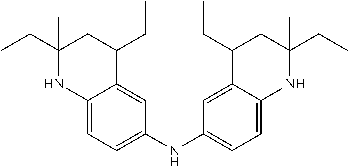 [SMILES: CCC1CC(C)(CC)Nc2ccc(Nc3ccc4c(c3)C(CC)CC(C)(CC)N4)cc21] |
| 121 | 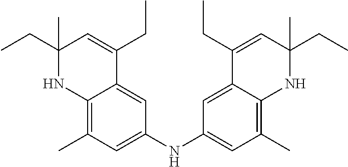 [SMILES: CCC1=CC(C)(CC)Nc2c(C)cc(Nc3cc(C)c4c(c3)C(CC)=CC(C)(CC)N4)cc21] |
| 122 | 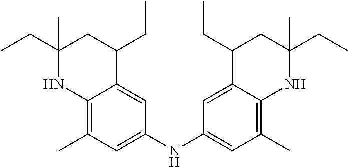 [SMILES: CCC1CC(C)(CC)Nc2c(C)cc(Nc3cc(C)c4c(c3)C(CC)CC(C)(CC)N4)cc21] |
| 123 | 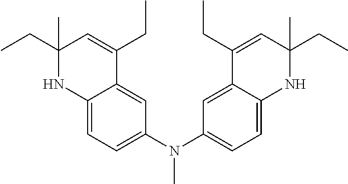 [SMILES: CCC1=CC(C)(CC)Nc2ccc(N(C)c3ccc4c(c3)C(CC)=CC(C)(CC)N4)cc21] |
| 124 | 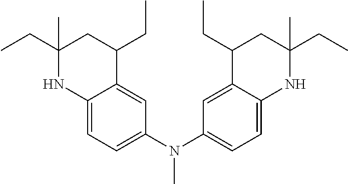 [SMILES: CCC1CC(C)(CC)Nc2ccc(N(C)c3ccc4c(c3)C(CC)CC(C)(CC)N4)cc21] |
| 125 | 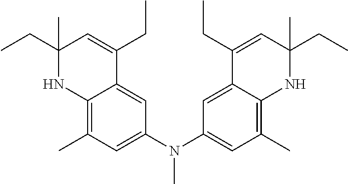 [SMILES: CCC1=CC(C)(CC)Nc2c(C)cc(N(C)c3cc(C)c4c(c3)C(CC)=CC(C)(CC)N4)cc21] |
| 126 | 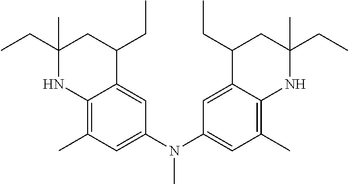 [SMILES: CCC1CC(C)(CC)Nc2c(C)cc(N(C)c3cc(C)c4c(c3)C(CC)CC(C)(CC)N4)cc21] |
| 127 | 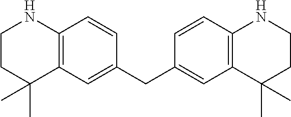 |
| 128 | 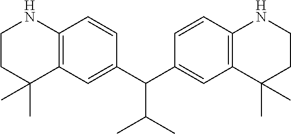 |
| 129 | 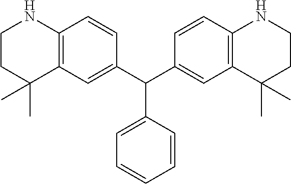 |
| 130 | 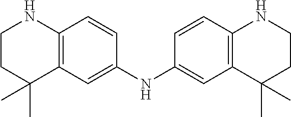 |
| 131 | 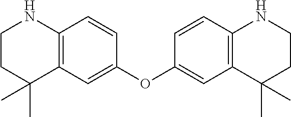 |
| 132 | 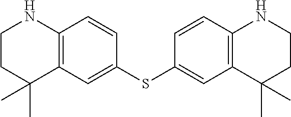 |
| 133 | 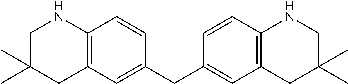 |
| 134 | 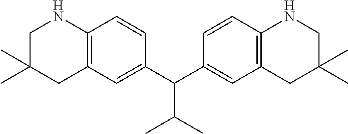 |
| 135 | 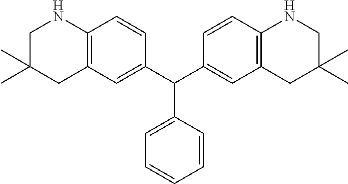 |
| 136 | 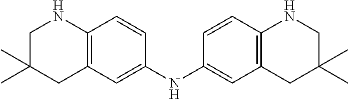 |
| 137 | 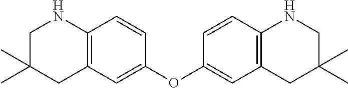 |
| 138 | 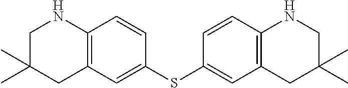 |
| 139 | 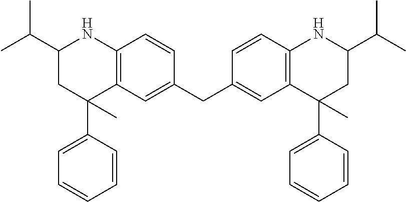 [SMILES: CC(C)C1CC(C)(c2ccccc2)c2cc(Cc3ccc4c(c3)C(C)(c3ccccc3)CC(C(C)C)N4)ccc2N1] |
| 140 | 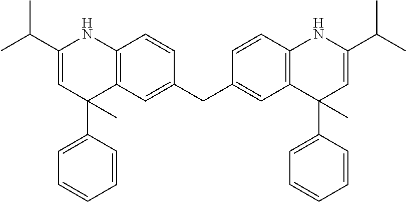 [SMILES: CC(C)C1=CC(C)(c2ccccc2)c2cc(Cc3ccc4c(c3)C(C)(c3ccccc3)C=C(C(C)C)N4)ccc2N1] |
| 141 | 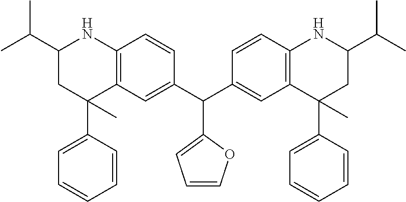 [SMILES: CC(C)C1CC(C)(c2ccccc2)c2cc(C(c3ccc4c(c3)C(C)(c3ccccc3)CC(C(C)C)N4)c3ccco3)ccc2N1] |
| 142 | 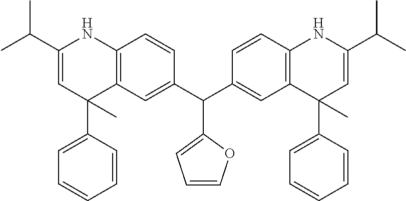 [SMILES: CC(C)C1=CC(C)(c2ccccc2)c2cc(C(c3ccc4c(c3)C(C)(c3ccccc3)C=C(C(C)C)N4)c3ccco3)ccc2N1] |
| 143 | 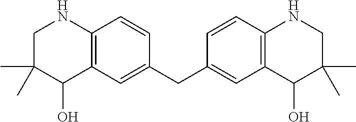 |
| 144 | 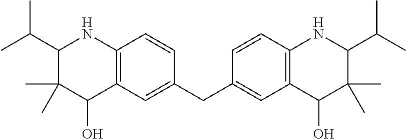 [SMILES: CC(C)C1Nc2ccc(Cc3ccc4c(c3)C(O)C(C)(C)C(C(C)C)N4)cc2C(O)C1(C)C] |
| 145 | 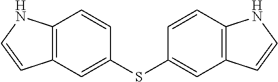 [SMILES: c1cc2cc(Sc3ccc4[nH]ccc4c3)ccc2[nH]1] |
| 146 | 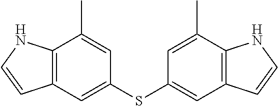 [SMILES: Cc1cc(Sc2cc(C)c3[nH]ccc3c2)cc2cc[nH]c12] |
| 147 | 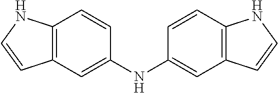 [SMILES: c1cc2cc(Nc3ccc4[nH]ccc4c3)ccc2[nH]1] |
| 148 | 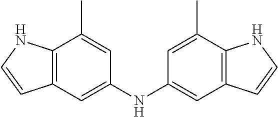 [SMILES: Cc1cc(Nc2cc(C)c3[nH]ccc3c2)cc2cc[nH]c12] |
| 149 | 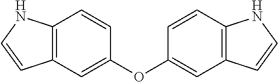 [SMILES: c1cc2cc(Oc3ccc4[nH]ccc4c3)ccc2[nH]1] |
| 150 | 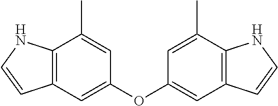 [SMILES: Cc1cc(Oc2cc(C)c3[nH]ccc3c2)cc2cc[nH]c12] |
| 151 | 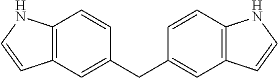 [SMILES: c1cc2cc(Cc3ccc4[nH]ccc4c3)ccc2[nH]1] |
| 152 | 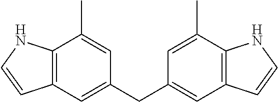 [SMILES: Cc1cc(Cc2cc(C)c3[nH]ccc3c2)cc2cc[nH]c12] |
| 153 | 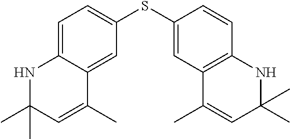 |
| 154 | 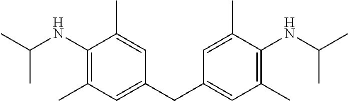 [SMILES: Cc1cc(Cc2cc(C)c(NC(C)C)c(C)c2)cc(C)c1NC(C)C] |
| 155 | 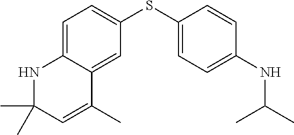 [SMILES: CC1=CC(C)(C)Nc2ccc(Sc3ccc(NC(C)C)cc3)cc21] |
| 156 | 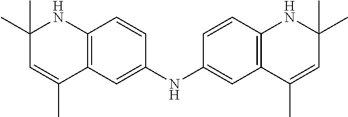 |
| 157 | 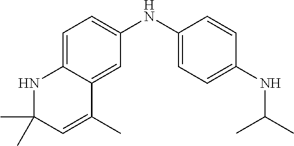 [SMILES: CC1=CC(C)(C)Nc2ccc(Nc3ccc(NC(C)C)cc3)cc21] |
| 158 | 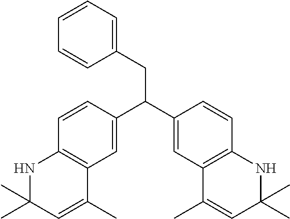 |
| 159 | 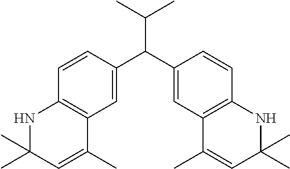 |
| 160 | 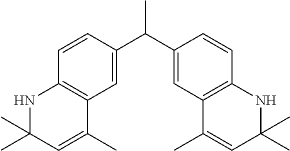 |
| 161 | 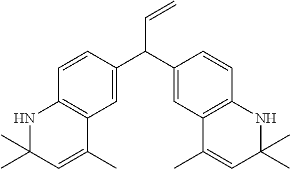 |
| 162 | 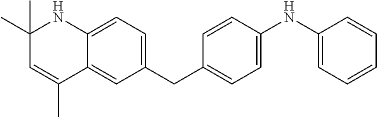 [SMILES: CC1=CC(C)(C)Nc2ccc(Cc3ccc(Nc4ccccc4)cc3)cc21] |
| 163 | 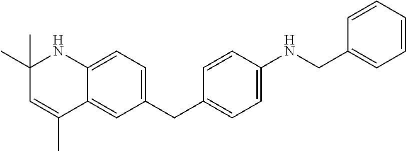 [SMILES: CC1=CC(C)(C)Nc2ccc(Cc3ccc(NCc4ccccc4)cc3)cc21] |
| 164 | 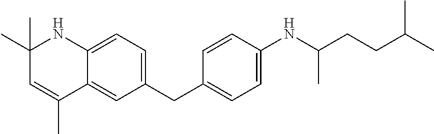 [SMILES: CC1=CC(C)(C)Nc2ccc(Cc3ccc(NC(C)CCC(C)C)cc3)cc21] |
| 165 | 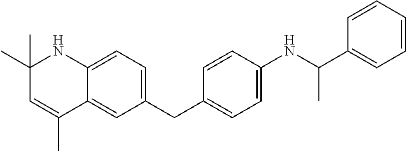 [SMILES: CC1=CC(C)(C)Nc2ccc(Cc3ccc(NC(C)c4ccccc4)cc3)cc21] |
| 166 | 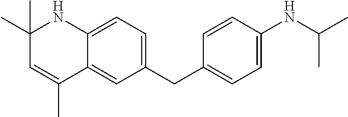 [SMILES: CC1=CC(C)(C)Nc2ccc(Cc3ccc(NC(C)C)cc3)cc21] |

Compounds of the Disclosure can be useful as antidegradants, e.g., antiozonants and/or antioxidants, as additives in lubricants, and as additives in combustible fuels.

Compounds of the Disclosure can be prepared by reacting a secondary aniline with an aldehyde in the presence of acid, as shown in Scheme 1.

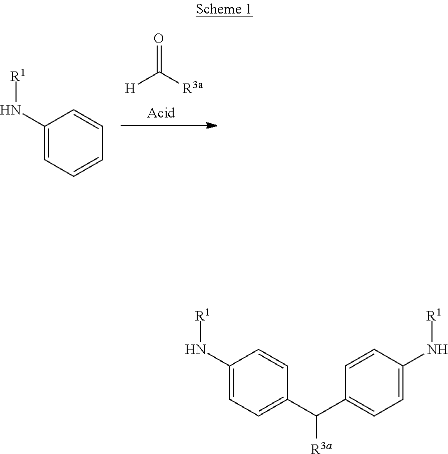

Compounds of the Disclosure can also be prepared by the reaction of 4,4′-alkylenedianilines with methyl ketones in the presence of acid, as shown in Scheme 2.

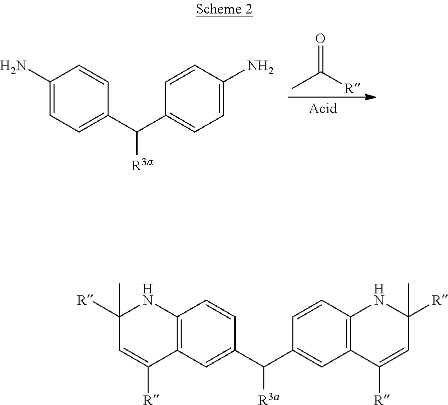

Compounds of the Disclosure can also be prepared by the reduction of the compounds obtained in the reaction described by Scheme 2, as shown in Scheme 3. Reduction may be carried out using any methods known in the art, such as exposure to hydrogen over a palladium on carbon (Pd/C) catalyst.

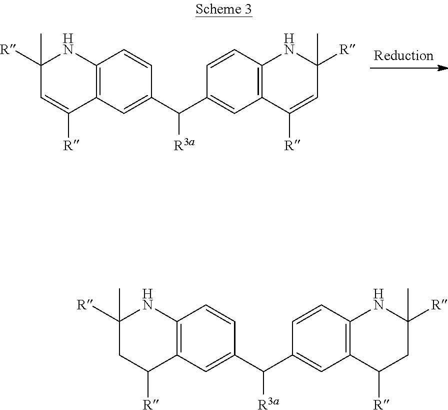

Compounds of the Disclosure can also be prepared, for example, by reacting a bis(aminophenyl)methane, bis(aminophenyl)ether, or bis(aminophenyl)sulfide with an alkene and an aldehyde or ketone in acetonitrile or other suitable solvents and a catalyst such as any suitable acid base, or I₂, i.e., via a Povarov reaction, as shown in Scheme 4, wherein R^{14a}, R^{14b}, R^{14c}, R^{14d}, R^{14e}, and R^{15f} are, for example, hydrogen or C₁-C₆ alkyl.

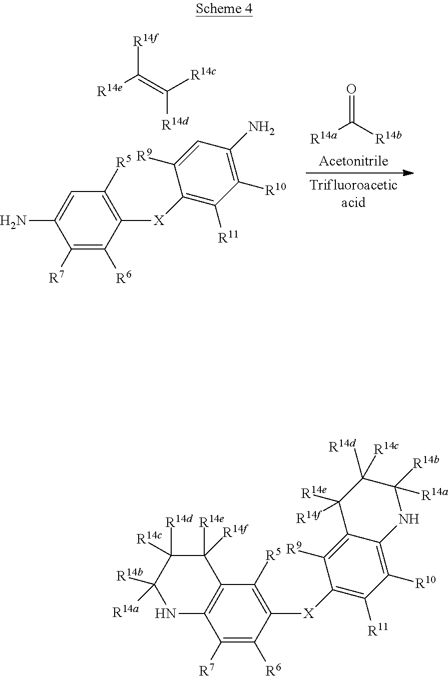

Compounds of the Disclosure can also be prepared by oxidizing the compound obtained in Scheme 4 by methods known in the art, as shown in Scheme 5.

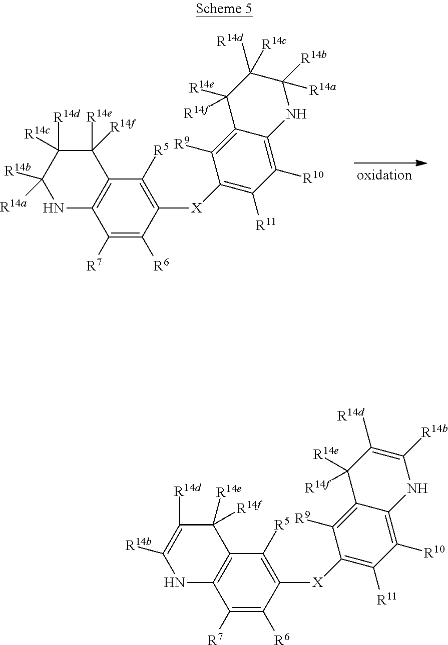

### Definitions

The term “alkyl” as used herein by itself or as part of another group refers to a straight- or branched-chain aliphatic hydrocarbon containing one to twelve carbon atoms, i.e., a C₁-C₁₂ alkyl, or the number of carbon atoms designated, e.g., C₁-C₃ alkyl such as methyl, ethyl, propyl, or isopropyl; a C₁-C₄ alkyl such as methyl, ethyl, propyl, isopropyl, n-butyl, isobutyl, sec-butyl, or t-butyl; and so on. In one embodiment the alkyl is a straight-chain alkyl. In another embodiment, the alkyl is a branched-chain alkyl. In one embodiment, the alkyl is a C₁-C₈ alkyl. In another embodiment, the alkyl is a C₁-C₆ alkyl. In another embodiment, the alkyl is a C₁-C₄ alkyl. In another embodiment, the alkyl is a C₁-C₃ alkyl. Non-limiting exemplary C₁-C₁₂ alkyl groups include methyl, ethyl, propyl, isopropyl, butyl, sec-butyl, tert-butyl, iso-butyl, 3-pentyl, hexyl, heptyl, octyl, nonyl, and decyl.

The term “alkenyl” as used herein by itself or as part of another group refers to an alkyl group containing one, two, or three carbon-to-carbon double bonds. In one embodiment, the alkenyl group is a C₂-C₆ alkenyl group. In another embodiment, the alkenyl group is a C₂-C₄ alkenyl group. In another embodiment, the alkenyl group has one carbon-to-carbon double bond. Non-limiting exemplary alkenyl groups include ethenyl, propenyl, isopropenyl, butenyl, sec-butenyl, pentenyl, and hexenyl.

The term “halo” or “halogen” as used herein by itself or as part of another group refers to —Cl, —F, —Br, or —I.

The term “nitro” as used herein by itself or as part of another group refers to —NO₂.

The term “cyano” as used herein by itself or as part of another group refers to —CN.

The term “hydroxy” as herein used by itself or as part of another group refers to —OH.

The term “amino” as used by itself or as part of another group refers to a radical of the formula —NR¹²R¹³, wherein R¹² and R¹³ are independently hydrogen, alkyl, or aryl.

In one embodiment, the amino is —NH₂.

In another embodiment, the amino is an “alkylamino,” i.e., an amino group wherein R¹² is C₁₋₆ alkyl and R¹³ is hydrogen. In one embodiment, R¹² is C₁-C₄ alkyl. Non-limiting exemplary alkylamino groups include —N(H)CH₃ and —N(H)CH₂CH₃.

In another embodiment, the amino is a “dialkylamino,” i.e., an amino group wherein R¹² and R¹³ are each independently C₁₋₆ alkyl. In one embodiment, R¹² and R¹³ are each independently C₁-C₄ alkyl. Non-limiting exemplary dialkylamino groups include —N(CH₃)₂ and —N(CH₃)CH₂CH(CH₃)₂.

In another embodiment, the amino is a “arylamino,” i.e., an amino group wherein R¹² is aryl and R¹³ is hydrogen. In one embodiment, R¹² is optionally substituted phenyl. In another embodiment, R¹² is phenyl, 2-methylphenyl, 3-methylphenyl, or 4-methylphenyl.

In another embodiment, the amino is a “diarylamino,” i.e., an amino group wherein R¹² and R¹³ are each independently aryl. In one embodiment, R¹² and R¹³ are phenyl.

The term “aryl” as used herein by itself or as part of another group refers to an aromatic ring system having six to fourteen carbon atoms, i.e., C₆-C₁₄ aryl. Non-limiting exemplary aryl groups include phenyl, naphthyl, phenanthryl, anthracyl, indenyl, azulenyl, biphenyl, biphenylenyl, and fluorenyl groups. In one embodiment, the aryl group is phenyl.

The term “alkylcarbonyl” as used herein by itself or as part of another group refers to a carbonyl group, i.e., —C(═O)—, substituted by an alkyl group. In one embodiment, the alkyl is a C₁-C₄ alkyl. A non-limiting exemplary alkylcarbonyl group is —COCH₃.

The term “haloalkyl” as used herein by itself or as part of another group refers to an alkyl substituted by one or more fluorine, chlorine, bromine, and/or iodine atoms. In one embodiment, the alkyl is substituted by one, two, or three fluorine and/or chlorine atoms. In another embodiment, the alkyl is substituted by one, two, or three fluorine atoms. In another embodiment, the alkyl is a C₁-C₆ alkyl and the resulting haloalkyl is referred to as a “C₁-C₆ haloalkyl.” In another embodiment, the alkyl is a C₁-C₄ alkyl and the resulting haloalkyl is referred to as a “C₁-C₄ haloalkyl.” In another embodiment, the alkyl group is a C₁ or C₂ alkyl. Non-limiting exemplary haloalkyl groups include fluoromethyl, difluoromethyl, trifluoromethyl, pentafluoroethyl, 1,1-difluoroethyl, 2,2-difluoroethyl, 2,2,2-trifluoroethyl, 3,3,3-trifluoropropyl, 4,4,4-trifluorobutyl, and trichloromethyl groups.

The term “alkoxy” as used herein by itself or as part of another group refers to an alkyl attached to a terminal oxygen atom. In one embodiment, the alkyl is a C₁-C₆ alkyl, and the resulting alkoxy is referred to as a “C₁-C₆ alkoxy.” In another embodiment, the alkyl is a C₁-C₄ alkyl group and thus the resulting alkoxy is referred to as a “C₁-C₄ alkoxy.” Non-limiting exemplary alkoxy groups include methoxy, ethoxy, and tert-butoxy.

The term “alkylthio” as used herein by itself or as part of another group refers to an alkyl group attached to a terminal sulfur atom. In one embodiment, the alkyl is a C₁- alkyl, and the resulting alkylthio is referred to as a “C₁-C₆ alkylthio.” In one embodiment, the alkyl group is a C₁-C₄ alkyl group and the resulting alkylthio is referred to as a “C₁-C₄ alkylthio.” Non-limiting exemplary alkylthio groups include —SCH₃, and —SCH₂CH₃.

The term “cycloalkyl” as used herein by itself or as part of another group refers to saturated and partially unsaturated, e.g., containing one or two double bonds, monocyclic, bicyclic, or tricyclic aliphatic hydrocarbons containing three to twelve carbon atoms, i.e., a C₃-C₁₂ cycloalkyl, or the number of carbons designated, e.g., a C₃-C₆ cycloalkyl such a cyclopropyl, cyclobutyl, cyclopentyl, and cyclohexyl. In one embodiment, the cycloalkyl is bicyclic, i.e., it has two rings. In another embodiment, the cycloalkyl is monocyclic, i.e., it has one ring. In another embodiment, the cycloalkyl is a C₃-C₈ cycloalkyl. In another embodiment, the cycloalkyl is a C₃-C₆ cycloalkyl. In another embodiment, the cycloalkyl is a C₅ cycloalkyl, i.e., cyclopentyl. In another embodiment, the cycloalkyl is a C₆ cycloalkyl, i.e., cyclohexyl. Non-limiting exemplary C₃-C₁₂ cycloalkyl groups include cyclopropyl, cyclobutyl, cyclopentyl, cyclohexyl, cycloheptyl, cyclooctyl, norbornyl, decalin, adamantyl, cyclohexenyl, and spiro[3.3]heptane.

The term “optionally substituted phenyl” as used herein by itself or as part of another group refers to phenyl that is either unsubstituted or substituted with one to five substitutents, wherein the substituents are each independently halo, nitro, cyano, hydroxyl, amino, (e.g., —NH₂, alkylamino, dialkylamino, arylamino, or diarylamino), alkoxy, alkylthio, alkylcarbonyl, alkyl, or cycloalkyl.

The term “heterocyclo” as used herein by itself or as part of another group refers to saturated and partially unsaturated, e.g., containing one or two double bonds, monocyclic, bicyclic, or tricyclic groups containing three to eighteen ring members, i.e., a 3- to 18-membered heterocyclo, comprising one, two, three, or four heteroatoms. Each heteroatom is independently oxygen, sulfur, or nitrogen. Each sulfur atom may be independently oxidized to give a sulfoxide, i.e., S(═O), or sulfone, i.e., S(═O)₂. The term heterocyclo includes groups wherein one or more —CH₂— groups is replaced with one or more —C(═O)— groups, including cyclic ureido groups such as imidazolidinyl-2-one, cyclic amide groups such as pyrrolidin-2-one or piperidin-2-one, and cyclic carbamate groups such as oxazolidinyl-2-one. The term heterocyclo also includes groups having fused optionally substituted aryl or optionally substituted heteroaryl groups such as indoline, indolin-2-one, 2,3-dihydro-1H-pyrrolo[2,3-c]pyridine, 2,3,4,5-tetrahydro-1H-benzo[d]azepine, or 1,3,4,5-tetrahydro-2H-benzo[d]azepin-2-one. In some embodiments, heterocyclo is a 6-membered ring comprising one nitrogen atom. The heterocyclo may be fused to the rest of the molecule to form a bicyclic group, e.g., 1,2-dihydroquinoline or 1,2,3,4-tetrahydroquinoline.

The term “optionally substituted heterocyclo” as used herein by itself or part of another group refers to a heterocyclo group that is either unsubstituted or substituted with one to four substituents, independently halo, nitro, cyano, hydroxyl, amino, (e.g., —NH₂, alkylamino, or dialkylamino), alkoxy, alkylthio, alkylcarbonyl, alkyl, or cycloalkyl.

The term “heteroaryl” as used herein by itself or as part of another group refers to monocyclic aromatic ring systems having five to six ring members, i.e., a 5- to 6-membered heteroaryl, comprising one, two, three, four, or five heteroatoms. Each heteroatom is independently oxygen, sulfur, or nitrogen. In one embodiment, the heteroaryl has three heteroatoms. In another embodiment, the heteroaryl has two heteroatoms. In another embodiment, the heteroaryl has one heteroatom. In another embodiment, the heteroaryl has 5 ring atoms, e.g., furyl, a 5-membered heteroaryl having four carbon atoms and one oxygen atom. In another embodiment, the heteroaryl has 6 ring atoms, e.g., pyridyl, a 6-membered heteroaryl having five carbon atoms and one nitrogen atom. Non-limiting exemplary heteroaryl groups include thienyl, furyl, pyranyl, 2H-pyrrolyl, pyrrolyl, imidazolyl, pyrazolyl, pyridyl, pyrazinyl, pyrimidinyl, pyridazinyl, thiazolyl, isothiazolyl, and isoxazolyl. In one embodiment, the heteroaryl is chosen from thienyl (e.g., thien-2-yl and thien-3-yl), furyl (e.g., 2-furyl and 3-furyl), pyrrolyl (e.g., 1H-pyrrol-2-yl and 1H-pyrrol-3-yl), imidazolyl (e.g., 2H-imidazol-2-yl and 2H-imidazol-4-yl), pyrazolyl (e.g., 1H-pyrazol-3-yl, 1H-pyrazol-4-yl, and 1H-pyrazol-5-yl), pyridyl (e.g., pyridin-2-yl, pyridin-3-yl, and pyridin-4-yl), pyrimidinyl (e.g., pyrimidin-2-yl, pyrimidin-4-yl, and pyrimidin-5-yl), thiazolyl (e.g., thiazol-2-yl, thiazol-4-yl, and thiazol-5-yl), isothiazolyl (e.g., isothiazol-3-yl, isothiazol-4-yl, and isothiazol-5-yl), oxazolyl (e.g., oxazol-2-yl, oxazol-4-yl, and oxazol-5-yl) and isoxazolyl (e.g., isoxazol-3-yl, isoxazol-4-yl, and isoxazol-5-yl). The term heteroaryl also includes N-oxides. A non-limiting exemplary N-oxide is pyridyl N-oxide.

The term “optionally substituted heteroaryl” as used herein by itself or as part of another group refers to a heteroaryl that is either unsubstituted or substituted with one to four substituents, wherein the substituents are independently halo, nitro, cyano, hydroxyl, amino, (e.g., —NH₂, alkylamino, or dialkylamino), alkoxy, alkylthio, alkylcarbonyl, alkyl, or cycloalkyl.

As used herein, the term “stereoisomers” is a general term for all isomers of an individual molecule that differ only in the orientation of their atoms in space. It includes enantiomers and isomers of compounds with more than one chiral center that are not mirror images of one another (diastereomers).

The term “chiral center” or “asymmetric carbon atom” refers to a carbon atom to which four different groups are attached.

The terms “enantiomer” and “enantiomeric” refer to a molecule that cannot be superimposed on its mirror image and hence is optically active wherein the enantiomer rotates the plane of polarized light in one direction and its mirror image compound rotates the plane of polarized light in the opposite direction.

The term “racemic” refers to a mixture of equal parts of enantiomers and which mixture is optically inactive.

The term “absolute configuration” refers to the spatial arrangement of the atoms of a chiral molecular entity (or group) and its stereochemical description, e.g., R or S.

The stereochemical terms and conventions used in the specification are meant to be consistent with those described in Pure & Appl. Chem 68:2193 (1996), unless otherwise indicated.

The term “enantiomeric excess” or “ee” refers to a measure for how much of one enantiomer is present compared to the other. For a mixture of R and S enantiomers, the percent enantiomeric excess is defined as |R−S|*100, where R and S are the respective mole or weight fractions of enantiomers in a mixture such that R+S=1. With knowledge of the optical rotation of a chiral substance, the percent enantiomeric excess is defined as ([α]_{obs}/[α]ₘₐₓ)*100, where [α]_{obs} is the optical rotation of the mixture of enantiomers and [α]ₘₐₓ is the optical rotation of the pure enantiomer. Determination of enantiomeric excess is possible using a variety of analytical techniques, including NMR spectroscopy, chiral column chromatography, or optical polarimetry.

In thin layer chromatography (TLC), the term R_{f} stands for retention factor. R_{f} is defined as the distance travelled by an individual component divided by the total distance travelled by the eluent. Its value is always between zero and one.

Salts and solvates, e.g., hydrates, of the Compounds of the Disclosure can also be used in the methods disclosed herein.

The present disclosure encompasses the preparation and use of salts of Compounds of the Disclosure. Salts of Compounds of the Disclosure can be prepared during the final isolation and purification of the compounds or separately by reacting the compound with an acid having a suitable cation. Salts of Compounds of the Disclosure can be acid addition salts formed with acceptable acids. Examples of acids which can be employed to form salts include inorganic acids such as nitric, boric, hydrochloric, hydrobromic, sulfuric, and phosphoric, and organic acids such as oxalic, maleic, succinic, and citric. Non-limiting examples of salts of compounds of the disclosure include, but are not limited to, the hydrochloride, hydrobromide, hydroiodide, sulfate, bisulfate, 2-hydroxyethansulfonate, phosphate, hydrogen phosphate, acetate, adipate, alginate, aspartate, benzoate, bisulfate, butyrate, camphorate, camphorsulfonate, digluconate, glycerolphosphate, hemisulfate, heptanoate, hexanoate, formate, succinate, fumarate, maleate, ascorbate, isethionate, salicylate, methanesulfonate, mesitylenesulfonate, naphthylenesulfonate, nicotinate, 2-naphthalenesulfonate, oxalate, pamoate, pectinate, persulfate, 3-phenylproprionate, picrate, pivalate, propionate, trichloroacetate, trifluoroacetate, phosphate, glutamate, bicarbonate, undecanoate, lactate, citrate, tartrate, gluconate, methanesulfonate, ethanedisulfonate, benzene sulfonate, and p-toluenesulfonate salts. In addition, available amino groups present in the compounds of the disclosure can be quaternized with methyl, ethyl, propyl, and butyl chlorides, bromides, and iodides; dimethyl, diethyl, dibutyl, and diamyl sulfates; decyl, lauryl, myristyl, and stearyl chlorides, bromides, and iodides; and benzyl and phenethyl bromides. In light of the foregoing, any reference to Compounds of the Disclosure appearing herein is intended to include Compounds of the Disclosure as well as salts, hydrates, or solvates thereof.

The present disclosure encompasses the preparation and use of solvates of Compounds of the Disclosure. The term “solvate” as used herein is a combination, physical association and/or solvation of a compound of the present disclosure with a solvent molecule such as, e.g., a disolvate, monosolvate or hemisolvate, where the ratio of solvent molecule to compound of the present disclosure is about 2:1, about 1:1 or about 1:2, respectively. This physical association involves varying degrees of ionic and covalent bonding, including hydrogen bonding. In certain instances, the solvate can be isolated, such as when one or more solvent molecules are incorporated into the crystal lattice of a crystalline solid. Thus, “solvate” encompasses both solution-phase and isolatable solvates. Compounds of the Disclosure can be present as solvated forms with a solvent, such as water, methanol, and ethanol, and it is intended that the disclosure includes both solvated and unsolvated forms of Compounds of the Disclosure.

One type of solvate is a hydrate. A “hydrate” relates to a particular subgroup of solvates where the solvent molecule is water. Preparation of solvates is known in the art. See, for example, M. Caira et al, J. Pharmaceut. Sci., 93(3):601-611 (2004), which describes the preparation of solvates of fluconazole with ethyl acetate and with water. Similar preparation of solvates, hemisolvates, hydrates, and the like are described by van Tonder et al., AAPS Pharm. Sci. Tech., 5(1):Article 12 (2004), and A. L. Bingham et al., Chem. Commun. 603-604 (2001). A typical, non-limiting, process of preparing a solvate would involve dissolving a Compound of the Disclosure in a desired solvent (organic, water, or a mixture thereof) at temperatures above 20° C. to about 25° C., then cooling the solution at a rate sufficient to form crystals, and isolating the crystals by known methods, e.g., filtration. Analytical techniques such as infrared spectroscopy can be used to confirm the presence of the solvent in a crystal of the solvate.

The use of the terms “a”, “an”, “the”, and similar referents in the context of describing the disclosure (especially in the context of the claims) are to be construed to cover both the singular and the plural, unless otherwise indicated. Recitation of ranges of values herein merely are intended to serve as a shorthand method of referring individually to each separate value falling within the range, unless otherwise indicated herein, and each separate value is incorporated into the specification as if it were individually recited herein. The use of any and all examples, or exemplary language (e.g., “such as”) provided herein, is intended to better illustrate the disclosure and is not a limitation on the scope of the disclosure unless otherwise claimed. No language in the specification should be construed as indicating any non-claimed element as essential to the practice of the disclosure.

The term “wt/wt %” as used herein refers to the mass of one component in a composition or blend, e.g., a composition comprising a Compound of the Disclosure and one or more elastomers; or a Composition of the Disclosure and one or more fillers; or a blend comprising two or more elastomers, etc., divided by the combined mass of all components in the composition or blend, times 100. For example, the wt/wt % of a Compound of the Disclosure in a composition comprising 1 kg of the compound, 1 kg natural rubber, and 2 kg of synthetic rubber, is 25 wt/wt % (1 kg/4 kg=0.25×100=25 wt/wt %). The wt/wt % of a Compound of the Disclosure in a composition comprising 1 kg of the compound and 1 kg of carbon black is 50 wt/wt % (1 kg/2 kg=0.20×100=50 wt/wt %). The wt/wt % of a Compound of the Disclosure in a composition comprising 1 kg of the compound, 1 kg natural rubber, 2 kg of synthetic rubber, and 1 kg of carbon black is 20 wt/wt % (1 kg/5 kg=0.20×100=20 wt/wt %). The wt/wt % of natural rubber in a blend of one or more elastomers comprising 20 kg natural rubber and 30 kg synthetic rubber is 40 wt/wt % (20 kg/50 kg=0.40×100=40 wt/wt %).

### Compositions and Methods of Use

In another embodiment, the disclosure provides compositions comprising:
(i) a Compound of the Disclosure; and
(ii) one or more elastomers; or
(iii) one or more fillers; or
(iv) one or more rubber chemicals; or
(v) one or more plasticizers; or
(vi) a second antidegradant; or
(vii) a combination of one or more elastomers, one or more fillers, one or more rubber chemicals, one or more plasticizers, and/or a second antidegradant.

In some embodiments, a Composition of the Disclosure comprises from about 15 wt/wt % to about 85 wt/wt % of a Compound of the Disclosure. In some embodiments, the composition comprises from about 1 wt/wt % to about 5 wt/wt %, from about 1 wt/wt % to about 15 wt/wt %, from about 1 wt/wt % to about 25 wt/wt %, from about 1 wt/wt % to about 35 wt/wt %, from about 1 wt/wt % to about 45 wt/wt %, from about 1 wt/wt % to about 55 wt/wt %, from about 1 wt/wt % to about 65 wt/wt %, from about 1 wt/wt % to about 75 wt/wt %, from about 1 wt/wt % to about 85 wt/wt %, from about 1 wt/wt % to about 95 wt/wt %, from about 5 wt/wt % to about 15 wt/wt %, from about 5 wt/wt % to about 25 wt/wt %, from about 5 wt/wt % to about 35 wt/wt %, from about 5 wt/wt % to about 45 wt/wt %, from about 5 wt/wt % to about 55 wt/wt %, from about 5 wt/wt % to about 65 wt/wt %, from about 5 wt/wt % to about 75 wt/wt %, from about 5 wt/wt % to about 85 wt/wt %, from about 5 wt/wt % to about 95 wt/wt %, from about 15 wt/wt % to about 25 wt/wt %, from about 15 wt/wt % to about 35 wt/wt %, from about 15 wt/wt % to about 45 wt/wt %, from about 15 wt/wt % to about 55 wt/wt %, from about 15 wt/wt % to about 65 wt/wt %, from about 15 wt/wt % to about 75 wt/wt %, from about 15 wt/wt % to about 95 wt/wt %, from about 25 wt/wt % to about 35 wt/wt %, from about 25 wt/wt % to about 45 wt/wt %, from about 25 wt/wt % to about 55 wt/wt %, from about 25 wt/wt % to about 65 wt/wt %, from about 25 wt/wt % to about 75 wt/wt %, from about 25 wt/wt % to about 85 wt/wt %, from about 25 wt/wt % to about 95 wt/wt %, from about 35 wt/wt % to about 45 wt/wt %, from about 35 wt/wt % to about 55 wt/wt %, from about 35 wt/wt % to about 65 wt/wt %, from about 35 wt/wt % to about 75 wt/wt %, from about 35 wt/wt % to about 85 wt/wt %, from about 35 wt/wt % to about 95 wt/wt %, from about 45 wt/wt % to about 55 wt/wt %, from about 45 wt/wt % to about 65 wt/wt %, from about 45 wt/wt % to about 75 wt/wt %, from about 45 wt/wt % to about 85 wt/wt %, from about 45 wt/wt % to about 95 wt/wt %, from about 55 wt/wt % to about 65 wt/wt %, from about 55 wt/wt % to about 75 wt/wt %, from about 55 wt/wt % to about 85 wt/wt %, from about 55 wt/wt % to about 95 wt/wt %, from about 65 wt/wt % to about 75 wt/wt %, from about 65 wt/wt % to about 85 wt/wt %, from about 65 wt/wt % to about 95 wt/wt %, from about 75 wt/wt % to about 85 wt/wt %, from about 75 wt/wt % to about 95 wt/wt % r from about 85 wt/wt % to about 95 wt/wt % of a Compound of the Disclosure. In some embodiments, the composition comprises from about 0.1 wt/wt % to about 0.5 wt/wt %, from about 0.1 wt/wt % to about 1 wt/wt %, from about 0.1 wt/wt % to about 1.5 wt/wt %, from about 0.1 wt/wt % to about 2 wt/wt %, from about 0.1 wt/wt % to about 2.5 wt/wt %, from about 0.1 wt/wt % to about 3 wt/wt %, from about 0.1 wt/wt % to about 3.5 wt/wt %, from about 0.1 wt/wt % to about 4 wt/wt %, from about 0.1 wt/wt % to about 4.5 wt/wt %, from about 0.1 wt/wt % to about 5 wt/wt %, from about 0.1 wt/wt % to about 6 wt/wt %, from about 0.1 wt/wt % to about 7 wt/wt %, from about 0.1 wt/wt % to about 8 wt/wt %, from about 0.1 wt/wt % to about 9 wt/wt %, from about 0.1 wt/wt % to about 10 wt/wt %, from about 0.5 wt/wt % to about 1 wt/wt %, from about 0.5 wt/wt % to about 1.5 wt/wt %, from about 0.5 wt/wt % to about 2 wt/wt %, from about 0.5 wt/wt % to about 2.5 wt/wt %, from about 0.5 wt/wt % to about 3 wt/wt %, from about 0.5 wt/wt % to about 3.5 wt/wt %, from about 0.5 wt/wt % to about 4 wt/wt %, from about 0.5 wt/wt % to about 4.5 wt/wt %, from about 0.5 wt/wt % to about 5 wt/wt %, from about 0.5 wt/wt % to about 6 wt/wt %, from about 0.5 wt/wt % to about 7 wt/wt %, from about 0.5 wt/wt % to about 8 wt/wt %, from about 0.5 wt/wt % to about 9 wt/wt %, from about 0.5 wt/wt % to about 10 wt/wt %, from about 1 wt/wt % to about 2 wt/wt %, from about 1 wt/wt % to about 2.5 wt/wt %, from about 1 wt/wt % to about 3 wt/wt %, from about 1 wt/wt % to about 3.5 wt/wt %, from about 1 wt/wt % to about 4 wt/wt %, from about 1 wt/wt % to about 4.5 wt/wt %, from about 1 wt/wt % to about 5 wt/wt %, from about 1 wt/wt % to about 6 wt/wt %, from about 1 wt/wt % to about 7 wt/wt %, from about 1 wt/wt % to about 8 wt/wt %, from about 1 wt/wt % to about 9 wt/wt %, from about 1 wt/wt % to about 10 wt/wt %, from about 1.5 wt/wt % to about 2 wt/wt %, from about 1.5 wt/wt % to about 2.5 wt/wt %, from about 1.5 wt/wt % to about 3 wt/wt %, from about 1.5 wt/wt % to about 3.5 wt/wt %, from about 1.5 wt/wt % to about 4 wt/wt %, from about 1.5 wt/wt % to about 4.5 wt/wt %, from about 1.5 wt/wt % to about 5 wt/wt %, from about 1.5 wt/wt % to about 6 wt/wt %, from about 1.5 wt/wt % to about 7 wt/wt %, from about 1.5 wt/wt % to about 8 wt/wt %, from about 1.5 wt/wt % to about 9 wt/wt %, from about 1.5 wt/wt % to about 10 wt/wt %, from about 2 wt/wt % to about 2.5 wt/wt %, from about 2 wt/wt % to about 3 wt/wt %, from about 2 wt/wt % to about 3.5 wt/wt %, from about 2 wt/wt % to about 4 wt/wt %, from about 2 wt/wt % to about 4.5 wt/wt %, from about 2 wt/wt % to about 5 wt/wt %, from about 2 wt/wt % to about 6 wt/wt %, from about 2 wt/wt % to about 7 wt/wt %, from about 2 wt/wt % to about 8 wt/wt %, from about 2 wt/wt % to about 9 wt/wt %, from about 2 wt/wt % to about 10 wt/wt %, from about 2.5 wt/wt % to about 3 wt/wt %, from about 2.5 wt/wt % to about 3.5 wt/wt %, from about 2.5 wt/wt % to about 4 wt/wt %, from about 2.5 wt/wt % to about 4.5 wt/wt %, from about 2.5 wt/wt % to about wt/wt %, from about 2.5 wt/wt % to about 6 wt/wt %, from about 2.5 wt/wt % to about 7 wt/wt %, from about 2.5 wt/wt % to about 8 wt/wt %, from about 2.5 wt/wt % to about 9 wt/wt %, from about 2.5 wt/wt % to about 10 wt/wt %, from about 3 wt/wt % to about 4 wt/wt %, from about 3 wt/wt % to about 4.5 wt/wt %, from about 3 wt/wt % to about wt/wt %, from about 3 wt/wt % to about 6 wt/wt %, from about 3 wt/wt % to about 7 wt/wt %, from about 3 wt/wt % to about 8 wt/wt %, from about 3 wt/wt % to about 9 wt/wt %, from about 3 wt/wt % to about 10 wt/wt %, from about 3.5 wt/wt % to about 4 wt/wt %, from about 3.5 wt/wt % to about 4.5 wt/wt %, from about 3.5 wt/wt % to about wt/wt %, from about 3.5 wt/wt % to about 6 wt/wt %, from about 3.5 wt/wt % to about 7 wt/wt %, from about 3.5 wt/wt % to about 8 wt/wt %, from about 3.5 wt/wt % to about 9 wt/wt %, from about 3.5 wt/wt % to about 10 wt/wt %, from about 4 wt/wt % to about 4.5 wt/wt %, from about 4 wt/wt % to about 7 wt/wt %, from about 4 wt/wt % to about 6 wt/wt %, from about 4 wt/wt % to about 7 wt/wt %, from about 4 wt/wt % to about 8 wt/wt %, from about 4 wt/wt % to about 9 wt/wt %, from about 4 wt/wt % to about 10 wt/wt %, from about 4.5 wt/wt % to about 7 wt/wt %, from about 4.5 wt/wt % to about 6 wt/wt %, from about 4.5 wt/wt % to about 7 wt/wt %, from about 4.5 wt/wt % to about 8 wt/wt %, from about 4.5 wt/wt % to about 9 wt/wt %, from about 4.5 wt/wt % to about 10 wt/wt %, from about 5 wt/wt % to about 6 wt/wt %, from about 5 wt/wt % to about 7 wt/wt %, from about 5 wt/wt % to about 8 wt/wt %, from about 5 wt/wt % to about 9 wt/wt %, from about 5 wt/wt % to about 10 wt/wt %, from about 6 wt/wt % to about 7 wt/wt %, from about 6 wt/wt % to about 8 wt/wt %, from about 6 wt/wt % to about 9 wt/wt %, from about 6 wt/wt % to about 10 wt/wt %, from about 7 wt/wt % to about 8 wt/wt %, from about 7 wt/wt % to about 9 wt/wt %, from about 8 wt/wt % to about 10 wt/wt %, from about 8 wt/wt % to about 9 wt/wt %, from about 8 wt/wt % to about 10 wt/wt %, or from about 9 wt/wt % to about 10 wt/wt % of a Compound of the Disclosure.

In some embodiments, a Composition of the Disclosure comprises about 50 wt/wt % of a Compound of the Disclosure. In some embodiments, the composition comprises about 1 wt/wt %, about 5 wt/wt %, about 10 wt/wt %, about 15 wt/wt %, about 20 wt/wt %, about 25 wt/wt %, about 30 wt/wt %, about 35 wt/wt %, about 40 wt/wt %, about 45 wt/wt %, about 55 wt/wt %, about 60 wt/wt %, about 65 wt/wt %, about 70 wt/wt %, about 75 wt/wt %, about 80 wt/wt %, about 85 wt/wt %, about 90 wt/wt %, or about 95 wt/wt % of a Compound of the Disclosure. In some embodiments, the composition comprises about 0.1 wt/wt %, about 0.5 wt/wt %, about 1 wt/wt %, about 1.5 wt/wt %, about 2 wt/wt %, about 2.5 wt/wt %, about 3 wt/wt %, about 3.5 wt/wt %, about 4 wt/wt %, about 4.5 wt/wt %, about 5 wt/wt %, about 5.5 wt/wt %, about 6 wt/wt %, about 6.5 wt/wt %, about 7 wt/wt %, about 7.5 wt/wt %, about 8 wt/wt %, about 8.5 wt/wt %, about 9 wt/wt %, or about 10 wt/wt % of a Compound of the Disclosure.

In some embodiments, the Composition of the Disclosure comprises Compound 1, Compound 2, Compound 4, Compound 5, or Compound 6, Compound 11, Compound 19, Compound 27, Compound 30, Compound 31, or Compound 32.

In another embodiment, a Composition of the Disclosure comprises a Compound of the Disclosure and one or more elastomers.

The term “elastomer” as used herein is a polymer with viscoelasticity (i.e., having both viscosity and elasticity) that typically has low intermolecular forces, low Young's modulus, and high failure strain. Elastomers can typically be cross-linked by heating in the presence of one or more cross-linking agents, a process called curing or vulcanization. Rubber is one type of elastomer. Non-limiting types of rubber include natural rubber (NR), synthetic rubber, and blends thereof. The term “natural rubber” as used herein refers to a naturally occurring elastomer that can be obtained from Hevea rubber trees. Non-limiting types of synthetic rubbers include unsaturated rubbers, saturated rubbers, rubbers with fluoro and fluoralkyl or fluoralkoxy substituent groups on the polymer chain (FKM), silicone rubbers (Q), and blends thereof. Non-limiting examples of unsaturated rubbers include polyisoprene rubber (IR), butyl rubber (IIR), polybutadiene rubber (BR), styrene-isoprene-butadiene rubber (SIBR), styrene butadiene rubber (SBR), nitrile butadiene rubber (NBR), chloroprene rubber (CR), ethylene propylene diene rubber (EPDM), and blends thereof. These unsaturated rubbers undergo cyclization and crosslinking reactions that lead to hardening of the aged part. As oxidation occurs, these vulcanizates harden and eventually become brittle products. Partial oxidation of vulcanizates leads to losses in performance when used in applications such as vehicle tire sidewalls. Saturated rubbers are rubbers that do not contain C═C unsaturation and include, but are not limited to, acrylic rubber (ACM), chlorinated polyethylene (CM), chlorosulfonated polyethylene (CSM), polychloromethyloxiran (CO), ethylene-ethyl acrylate copolymer (EAM), epichlorohydrin rubber (ECO), ethylene propylene rubber (EPM), ethylenevinylacetate copolymer (EVM), rubbers with fluoro and fluoralkyl or fluoralkoxy substituent groups on the polymer chain (FKM), silicone rubber (Q), and blends thereof.

In some embodiments, the natural rubber comprises rubber derived from an alternative rubber plant. The term “natural rubber comprises rubber derived from an alternative rubber plant” as used herein refers to a naturally occurring elastomer that can be obtained from “non-Hevea” sources. In some embodiments, the alternative rubber plant is Parthenium argentatum (guayule) or Taraxacum kok-saghyz (Russian dandelion).

In some embodiments, the one or more elastomers further comprises recycled rubber. The term “recycled rubber” as used herein refers to an elastomer that has been reclaimed from scrap materials such as used tires.

In some embodiments, a Composition of the Disclosure comprises from about 15 wt/wt % to about 85 wt/wt % of one or more elastomers. In some embodiments, the composition comprises from about 1 wt/wt % to about 5 wt/wt %, from about 1 wt/wt % to about 15 wt/wt %, from about 1 wt/wt % to about 25 wt/wt %, from about 1 wt/wt % to about 35 wt/wt %, from about 1 wt/wt % to about 45 wt/wt %, from about 1 wt/wt % to about 55 wt/wt %, from about 1 wt/wt % to about 65 wt/wt %, from about 1 wt/wt % to about 75 wt/wt %, from about 1 wt/wt % to about 85 wt/wt %, from about 1 wt/wt % to about 95 wt/wt %, from about 5 wt/wt % to about 15 wt/wt %, from about 5 wt/wt % to about 25 wt/wt %, from about 5 wt/wt % to about 35 wt/wt %, from about 5 wt/wt % to about 45 wt/wt %, from about 5 wt/wt % to about 55 wt/wt %, from about 5 wt/wt % to about 65 wt/wt %, from about 5 wt/wt % to about 75 wt/wt %, from about 5 wt/wt % to about 85 wt/wt %, from about 5 wt/wt % to about 95 wt/wt %, from about 15 wt/wt % to about 25 wt/wt %, from about 15 wt/wt % to about 35 wt/wt %, from about 15 wt/wt % to about 45 wt/wt %, from about 15 wt/wt % to about 55 wt/wt %, from about 15 wt/wt % to about 65 wt/wt %, from about 15 wt/wt % to about 75 wt/wt %, from about 15 wt/wt % to about 95 wt/wt %, from about 25 wt/wt % to about 35 wt/wt %, from about 25 wt/wt % to about 45 wt/wt %, from about 25 wt/wt % to about 55 wt/wt %, from about 25 wt/wt % to about 65 wt/wt %, from about 25 wt/wt % to about 75 wt/wt %, from about 25 wt/wt % to about 85 wt/wt %, from about 25 wt/wt % to about 95 wt/wt %, from about 35 wt/wt % to about 45 wt/wt %, from about 35 wt/wt % to about 55 wt/wt %, from about 35 wt/wt % to about 65 wt/wt %, from about 35 wt/wt % to about 75 wt/wt %, from about 35 wt/wt % to about 85 wt/wt %, from about 35 wt/wt % to about 95 wt/wt %, from about 45 wt/wt % to about 55 wt/wt %, from about 45 wt/wt % to about 65 wt/wt %, from about 45 wt/wt % to about 75 wt/wt %, from about 45 wt/wt % to about 85 wt/wt %, from about 45 wt/wt % to about 95 wt/wt %, from about 55 wt/wt % to about 65 wt/wt %, from about 55 wt/wt % to about 75 wt/wt %, from about 55 wt/wt % to about 85 wt/wt %, from about 55 wt/wt % to about 95 wt/wt %, from about 65 wt/wt % to about 75 wt/wt %, from about 65 wt/wt % to about 85 wt/wt %, from about 65 wt/wt % to about 95 wt/wt %, from about 75 wt/wt % to about 85 wt/wt %, from about 75 wt/wt % to about 95 wt/wt % or from about 85 wt/wt % to about 95 wt/wt % of one or more elastomers.

In some embodiments, a Composition of the Disclosure comprises about 50 wt/wt % of one or more elastomers. In some embodiments, the composition comprises about 1 wt/wt %, about 5 wt/wt %, about 10 wt/wt %, about 15 wt/wt %, about 20 wt/wt %, about 25 wt/wt %, about 30 wt/wt %, about 35 wt/wt %, about 40 wt/wt %, about 45 wt/wt %, about 55 wt/wt %, about 60 wt/wt %, about 65 wt/wt %, about 70 wt/wt %, about 75 wt/wt %, about 80 wt/wt %, about 85 wt/wt %, about 90 wt/wt %, or about 95 wt/wt % f one or more elastomers.

The term “phr” as used herein refers to parts per hundred parts of rubber by weight. The parts by weight of individual components are based on 100 parts by weight of the total mass of the one or more elastomers present in the composition.

In some embodiments, a Composition of the Disclosure comprises from about 1 phr to about 5 phr of a Compound of the Disclosure. In some embodiments, the composition comprises from about 0.01 phr to about 0.1 phr, from about 0.01 phr to about 0.5 phr, from about 0.01 phr to about 1 phr, from about 0.01 phr to about 2 phr, from about 0.01 phr to about 3 phr, from about 0.01 phr to about 4 phr, from about 0.01 phr to about 5 phr, from about 0.01 phr to about 7.5 phr, from about 0.01 phr to about 10 phr, from about 0.01 phr to about 20 phr, from about 0.1 phr to about 0.5 phr, from about 0.1 phr to about 1 phr, from about 0.1 phr to about 2 phr, from about 0.1 phr to about 3 phr, from about 0.1 phr to about 4 phr, from about 0.1 phr to about 5 phr, from about 0.1 phr to about 7.5 phr, from about 0.1 phr to about 10 phr, from about 0.1 phr to about 20 phr, from about 1 phr to about 2 phr, from about 1 phr to about 3 phr, from about 1 phr to about 4 phr, from about 1 phr to about 7.5 phr, from about 1 phr to about 10 phr, from about 1 phr to about 20 phr, from about 2 phr to about 3 phr, from about 2 phr to about 4 phr, from about 2 phr to about 5 phr, from about 2 phr to about 7.5 phr, from about 2 phr to about 10 phr, from about 2 phr to about 20 phr, from about 3 phr to about 4 phr, from about 3 phr to about 5 phr, from about 3 phr to about 7.5 phr, from about 3 phr to about 10 phr, from about 3 phr to about 20 phr, from about 4 phr to about 5 phr, from about 4 phr to about 7.5 phr, from about 4 phr to about 10 phr, from about 4 phr to about 20 phr, from about 5 phr to about 7.5 phr, from about 5 phr to about 10 phr, from about 5 phr to about 20 phr, from about 7.5 phr to about 10 phr, from about 7.5 phr to about 20 phr, or from about 10 phr to about 20 phr of a Compound of the Disclosure.

In some embodiments, a Composition of the Disclosure comprises about 3 phr of a Compound of the Disclosure. In some embodiments, the composition comprises about 0.01 phr, about 0.1 phr, about 0.5 phr, about 1 phr, about 2 phr, about 3 phr, about 4 phr, about 5 phr, about 7.5 phr, about 10 phr, or about 20 phr of a Compound of the Disclosure.

In some embodiments, a Composition of the Disclosure comprises a Compound of the Disclosure and one or more fillers.

The term “filler” as used herein is a substance that reinforces an elastomeric composition or gives an elastomeric composition other properties, including but not limited to expanding the volume of the composition. Non-limiting examples of fillers include carbon black, silica, kaolin, calcium silicate, talc, carbon nanotubes (CNT), carbon fibers (HCF), graphite, graphenes, aluminosilicates, starch, and fibers, and combinations thereof.

In some embodiments, the filler is derived from natural sources. For example, silica may be derived from rice husks.

In some embodiments, a Composition of the Disclosure comprises from about 15 wt/wt % to about 85 wt/wt % of one or more fillers. In some embodiments, the composition comprises from about 1 wt/wt % to about 5 wt/wt %, from about 1 wt/wt % to about 15 wt/wt %, from about 1 wt/wt % to about 25 wt/wt %, from about 1 wt/wt % to about 35 wt/wt %, from about 1 wt/wt % to about 45 wt/wt %, from about 1 wt/wt % to about 55 wt/wt %, from about 1 wt/wt % to about 65 wt/wt %, from about 1 wt/wt % to about 75 wt/wt %, from about 1 wt/wt % to about 85 wt/wt %, from about 1 wt/wt % to about 95 wt/wt %, from about 5 wt/wt % to about 15 wt/wt %, from about 5 wt/wt % to about 25 wt/wt %, from about 5 wt/wt % to about 35 wt/wt %, from about 5 wt/wt % to about 45 wt/wt %, from about 5 wt/wt % to about 55 wt/wt %, from about 5 wt/wt % to about 65 wt/wt %, from about 5 wt/wt % to about 75 wt/wt %, from about 5 wt/wt % to about 85 wt/wt %, from about 5 wt/wt % to about 95 wt/wt %, from about 15 wt/wt % to about 25 wt/wt %, from about 15 wt/wt % to about 35 wt/wt %, from about 15 wt/wt % to about 45 wt/wt %, from about 15 wt/wt % to about 55 wt/wt %, from about 15 wt/wt % to about 65 wt/wt %, from about 15 wt/wt % to about 75 wt/wt %, from about 15 wt/wt % to about 95 wt/wt %, from about 25 wt/wt % to about 35 wt/wt %, from about 25 wt/wt % to about 45 wt/wt %, from about 25 wt/wt % to about 55 wt/wt %, from about 25 wt/wt % to about 65 wt/wt %, from about 25 wt/wt % to about 75 wt/wt %, from about 25 wt/wt % to about 85 wt/wt %, from about 25 wt/wt % to about 95 wt/wt %, from about 35 wt/wt % to about 45 wt/wt %, from about 35 wt/wt % to about 55 wt/wt %, from about 35 wt/wt % to about 65 wt/wt %, from about 35 wt/wt % to about 75 wt/wt %, from about 35 wt/wt % to about 85 wt/wt %, from about 35 wt/wt % to about 95 wt/wt %, from about 45 wt/wt % to about 55 wt/wt %, from about 45 wt/wt % to about 65 wt/wt %, from about 45 wt/wt % to about 75 wt/wt %, from about 45 wt/wt % to about 85 wt/wt %, from about 45 wt/wt % to about 95 wt/wt %, from about 55 wt/wt % to about 65 wt/wt %, from about 55 wt/wt % to about 75 wt/wt %, from about 55 wt/wt % to about 85 wt/wt %, from about 55 wt/wt % to about 95 wt/wt %, from about 65 wt/wt % to about 75 wt/wt %, from about 65 wt/wt % to about 85 wt/wt %, from about 65 wt/wt % to about 95 wt/wt %, from about 75 wt/wt % to about 85 wt/wt %, from about 75 wt/wt % to about 95 wt/wt % or from about 85 wt/wt % to about 95 wt/wt % of one or more fillers.

In some embodiments, a Composition of the Disclosure comprises about 50 wt/wt % of one or more fillers. In some embodiments, the composition comprises about 1 wt/wt %, about 5 wt/wt %, about 10 wt/wt %, about 15 wt/wt %, about 20 wt/wt %, about 25 wt/wt %, about 30 wt/wt %, about 35 wt/wt %, about 40 wt/wt %, about 45 wt/wt %, about 55 wt/wt %, about 60 wt/wt %, about 65 wt/wt %, about 70 wt/wt %, about 75 wt/wt %, about 80 wt/wt %, about 85 wt/wt %, about 90 wt/wt %, or about 95 wt/wt % f one or more fillers.

In some embodiments, a Composition of the Disclosure comprises from about 30 phr to about 500 phr of one or more fillers. In some embodiments, the composition comprises from about 30 phr to about 50 phr, from about 30 phr to about 100 phr, from about 30 phr to about 150 phr, from about 30 phr to about 200 phr, from about 30 phr to about 250 phr, from about 30 phr to about 300 phr, from about 30 phr to about 350 phr, from about 30 phr to about 400 phr, from about 30 phr to about 450 phr, from about 30 phr to about 500 phr, from about 50 phr to about 100 phr, from about 50 phr to about 150 phr, from about 50 phr to about 200 phr, from about 50 phr to about 250 phr, from about 50 phr to about 300 phr, from about 50 phr to about 350 phr, from about 50 phr to about 400 phr, from about 50 phr to about 450 phr, from about 50 phr to about 500 phr, from about 100 phr to about 150 phr, from about 100 phr to about 200 phr, from about 100 phr to about 250 phr, from about 100 phr to about 300 phr, from about 100 phr to about 350 phr, from about 100 phr to about 400 phr, from about 100 phr to about 450 phr, from about 100 phr to about 500 phr, from about 150 phr to about 200 phr, from about 150 phr to about 250 phr, from about 150 phr to about 300 phr, from about 150 phr to about 350 phr, from about 150 phr to about 400 phr, from about 150 phr to about 450 phr, from about 150 phr to about 500 phr, from about 200 phr to about 250 phr, from about 200 phr to about 300 phr, from about 200 phr to about 350 phr, from about 200 phr to about 400 phr, from about 200 phr to about 450 phr, from about 200 phr to about 500 phr, from about 250 phr to about 300 phr, from about 250 phr to about 350 phr, from about 250 phr to about 400 phr, from about 250 phr to about 450 phr, from about 250 phr to about 500 phr, from about 300 phr to about 350 phr, from about 300 phr to about 400 phr, from about 300 phr to about 450 phr, from about 300 phr to about 500 phr, from about 350 phr to about 400 phr, from about 350 phr to about 450 phr, from about 350 phr to about 500 phr, from about 400 phr to about 450 phr, from about 400 phr to about 500 phr, or from about 450 phr to about 500 phr of one or more fillers.

In some embodiments, a Composition of the Disclosure comprises about 300 phr of one or more fillers. In some embodiments, the composition comprises about 30 phr, about 50 phr, about 100 phr, about 150 phr, about 200 phr, about 250 phr, about 350 phr, about 400 phr, about 450 phr, or about 500 phr of one or more fillers.

In some embodiments, a Composition of the Disclosure comprises a Compound of the Disclosure and one or more rubber chemicals. The term “rubber chemicals” as used herein refers to a compound or substance used to facilitate the vulcanization of rubber. Rubber chemicals include, but are not limited to, vulcanizing agents, accelerators, activators, and pre-vulcanization inhibitors.

The term “vulcanization” as used herein refers to a process wherein cross-links are formed between elastomers to effect changes in the material properties of elastomers. In particular, vulcanization typically increases the rigidity and durability of elastomers. Vulcanization is carried out at room temperature or at elevated temperatures, depending on the nature of the elastomer(s), filler(s), and rubber chemical(s) being used. The term “curing” is also used in the art to describe this process.

The term “vulcanizing agent” as used herein refers to any substance that enables cross-linking between elastomers. Vulcanizing agents can enable cross-linking between separate polymer chains of an elastomer by various mechanisms, including, but not limited to, by formation of covalent bonds between the vulcanizing agent and two or more separate polymer chains or by generating radical species on separate polymer chains that can combine to form covalent bonds between the two polymer chains. Non-limiting examples of vulcanizing agents include sulfur, peroxides, vulcanized vegetable oil, factices and resins. Non-limiting examples of sulfur include octasulfur (S₈), cyclododecasulfur (S₁₂), and polymeric sulfur. Non-limiting examples of peroxides include benzoyl peroxide, dicumyl peroxide (DC), 2,5-dimethyl-2,5-di-(tert-butylperoxy)-3-hexyne (2,5 Tri), 2,5-dimethyl-2,5-di(tert-butylperoxy)hexane (DDPH), di-(2-tert-butylperoxyisopropyl)benzene (VC), butyl-4,4-di-(tert-butylperoxy)valerate (VAL), and 1,1-di(tert-butylperoxy)-3,3,5-trimethylcyclohexane (TMC). Nonlimiting examples of resins include bonding resins. The term “bonding resin” as used herein refers to a chemical such as resorcinol formaldehyde resins and phenolic resins that reacts with methylene donors (such as hexamethylenetetramine (HMTA) or hexamethoxymethyl melamine (HMMM)) to promote adhesion.

The term “accelerator” as used herein refers to any substance that increases the kinetics of vulcanization. In some embodiments, accelerators enable vulcanization to be performed at lower temperatures and/or to use the vulcanization agent, e.g., sulfur, more efficiently. Non-limiting examples of accelerators include guanidines, thiazoles, sulfenamides, thiurams, dithiocarbamates, xanthates, and thiophosphates. Non-limiting examples of guanidines include diphenylguanidine (DPG). Non-limiting examples of thiazoles include 2-mercaptobenzothiazole (MBT), zinc 2-mercaptobenzothiazole (ZMBT), mercaptobenzothiazole disulfide (MBTS), and N-tert-butyl-2-benzothiazole sulfenimide (TBSI). Non-limiting examples of sulfenamides include N-tert-butyl-2-benzothiazylsulfenamide (TBBS), N-cyclohexylbenzothiazol-2-sulfenamide (CBS), dicyclohexyl-2-benzothiazolesulfenamide (DCBS), N-oxydiethylene benzothiazole sulfenamide (OBTS), N-oxydiethylenethiocarbamyl-N′-oxydiethylene sulfenamide (OTOS), and thiocarbamyl sulfenamide. Non-limiting examples of thiurams include dimethylcarbamothioic dithioperoxyanhydride (thiram), dipentamethylene thiuram tetrasulfide (DPIT), tetrabenzyl thiuram disulfide (TBzTD), tetraethylthiuram disulfide (TETD), tetramethylthiuram disulfide (TMTD), and tetramethylthiuram monosulfide (TMTM). Non-limiting examples of dithiocarbamates include zinc dimethyldithiocarbamate (ZDMC), zinc diethyldithiocarbamate (ZDEC), zinc dibutyldithiocarbamate (ZDBC), nickel dibutyldithiocarbamate (NDBC), sodium dibenzyldithiocarbamate (SBEC), sodium diethyldithiocarbamate (SDEC), tellurium diethyldithiocarbamate (TDEC), and zinc dibenzyldithiocarbamate (ZEBC).

The term “activator” as used herein refers to any substance that activates a vulcanizing agent and enables it to cross-link elastomers as described above. Activators may act via various mechanisms, including, but not limited to, by forming chemical complexes with accelerators or by coordinating to sulfur (when sulfur is used as a vulcanizing agent). Non-limiting examples of activators include metal oxides, acids, and metal complexes. Non-limiting examples of metal oxides include zinc oxide, magnesium oxide, and lead oxide. Non-limiting examples of acids include stearic acid and lauric acid. Non limiting examples of metal complexes include zinc ethylhexanoate.

The term “pre-vulcanization inhibitor” as used herein refers to compounds that delay the onset and/or the rate of vulcanization. These compounds are also referred to as “retarders.” Non-limiting examples of pre-vulcanization inhibitors include N-(cyclohexylthio)phthalimide (CTP), benzoic anhydride, salicylic anhydride, and phthalic anhydride.

In some embodiments, a Composition of the Disclosure comprises from about 15 wt/wt % to about 85 wt/wt % of one or more rubber chemicals. In some embodiments, the composition comprises from about 1 wt/wt % to about 5 wt/wt %, from about 1 wt/wt % to about 15 wt/wt %, from about 1 wt/wt % to about 25 wt/wt %, from about 1 wt/wt % to about 35 wt/wt %, from about 1 wt/wt % to about 45 wt/wt %, from about 1 wt/wt % to about 55 wt/wt %, from about 1 wt/wt % to about 65 wt/wt %, from about 1 wt/wt % to about 75 wt/wt %, from about 1 wt/wt % to about 85 wt/wt %, from about 1 wt/wt % to about 95 wt/wt %, from about 5 wt/wt % to about 15 wt/wt %, from about 5 wt/wt % to about 25 wt/wt %, from about 5 wt/wt % to about 35 wt/wt %, from about 5 wt/wt % to about 45 wt/wt %, from about 5 wt/wt % to about 55 wt/wt %, from about 5 wt/wt % to about 65 wt/wt %, from about 5 wt/wt % to about 75 wt/wt %, from about 5 wt/wt % to about 85 wt/wt %, from about 5 wt/wt % to about 95 wt/wt %, from about 15 wt/wt % to about 25 wt/wt %, from about 15 wt/wt % to about 35 wt/wt %, from about 15 wt/wt % to about 45 wt/wt %, from about 15 wt/wt % to about 55 wt/wt %, from about 15 wt/wt % to about 65 wt/wt %, from about 15 wt/wt % to about 75 wt/wt %, from about 15 wt/wt % to about 95 wt/wt %, from about 25 wt/wt % to about 35 wt/wt %, from about 25 wt/wt % to about 45 wt/wt %, from about 25 wt/wt % to about 55 wt/wt %, from about 25 wt/wt % to about 65 wt/wt %, from about 25 wt/wt % to about 75 wt/wt %, from about 25 wt/wt % to about 85 wt/wt %, from about 25 wt/wt % to about 95 wt/wt %, from about 35 wt/wt % to about 45 wt/wt %, from about 35 wt/wt % to about 55 wt/wt %, from about 35 wt/wt % to about 65 wt/wt %, from about 35 wt/wt % to about 75 wt/wt %, from about 35 wt/wt % to about 85 wt/wt %, from about 35 wt/wt % to about 95 wt/wt %, from about 45 wt/wt % to about 55 wt/wt %, from about 45 wt/wt % to about 65 wt/wt %, from about 45 wt/wt % to about 75 wt/wt %, from about 45 wt/wt % to about 85 wt/wt %, from about 45 wt/wt % to about 95 wt/wt %, from about 55 wt/wt % to about 65 wt/wt %, from about 55 wt/wt % to about 75 wt/wt %, from about 55 wt/wt % to about 85 wt/wt %, from about 55 wt/wt % to about 95 wt/wt %, from about 65 wt/wt % to about 75 wt/wt %, from about 65 wt/wt % to about 85 wt/wt %, from about 65 wt/wt % to about 95 wt/wt %, from about 75 wt/wt % to about 85 wt/wt %, from about 75 wt/wt % to about 95 wt/wt % or from about 85 wt/wt % to about 95 wt/wt % of one or more rubber chemicals.

In some embodiments, a Composition of the Disclosure comprises about 15 wt/wt % of one or more rubber chemicals. In some embodiments, the composition comprises about 1 wt/wt %, about 5 wt/wt %, about 10 wt/wt %, about 20 wt/wt %, about 25 wt/wt %, about 30 wt/wt %, about 35 wt/wt %, about 40 wt/wt %, about 45 wt/wt %, about 50 wt/wt %, about 55 wt/wt %, about 60 wt/wt %, about 65 wt/wt %, about 70 wt/wt %, about 75 wt/wt %, about 80 wt/wt %, about 85 wt/wt %, about 90 wt/wt %, or about 95 wt/wt % of one or more rubber chemicals.

In some embodiments, a Composition of the Disclosure comprises from about 1 phr to about 20 phr of one or more rubber chemicals. In some embodiments, the composition comprises from about 0.1 phr to about 1 phr, from about 0.1 phr to about 5 phr, from about 0.1 phr to about 10 phr, from about 0.1 phr to about 15 phr, from about 0.1 phr to about 20 phr, from about 0.1 phr to about 25 phr, from about 0.1 phr to about 30 phr, from about 0.1 phr to about 35 phr, from about 0.1 phr to about 40 phr, from about 1 phr to about 5 phr, from about 1 phr to about 10 phr, from about 1 phr to about 15 phr, from about 1 phr to about 25 phr, from about 1 phr to about 30 phr, from about 1 phr to about 35 phr, from about 1 phr to about 40 phr, from about 5 phr to about 10 phr, from about 5 phr to about 15 phr, from about 5 phr to about 20 phr, from about 5 phr to about 25 phr, from about 5 phr to about 30 phr, from about 5 phr to about 35 phr, from about 5 phr to about 40 phr, from about 10 phr to about 15 phr, from about 10 phr to about 20 phr, from about 10 phr to about 25 phr, from about 10 phr to about 30 phr, from about 10 phr to about 35 phr, from about 10 phr to about 40 phr, from about 15 phr to about 20 phr, from about 15 phr to about 25 phr, from about 15 phr to about 30 phr, from about 15 phr to about 35 phr, from about 15 phr to about 40 phr, from about 20 phr to about 25 phr, from about 20 phr to about 30 phr, from about 20 phr to about 35 phr, from about 20 phr to about 40 phr, from about 25 phr to about 30 phr, from about 25 phr to about 35 phr, from about 25 phr to about 40 phr, from about 30 phr to about 35 phr, from about 30 phr to about 40 phr, or from about 35 phr to about 40 phr of one or more rubber chemicals.

In some embodiments, a Composition of the Disclosure comprises about 10 phr of one or more rubber chemicals. In some embodiments, the composition comprises about 0.1 phr, about 1 phr, about 5 phr, about 15 phr, about 20 phr, about 25 phr, about 30 phr, about 35 phr, or about 40 phr of one or more rubber chemicals.

The term “plasticizer” as used herein refers to a processing aid used to reduce the viscosity, increase the plasticity, and/or extend the volume of a composition. Plasticizers facilitate the process of mixing and forming a composition comprising an elastomer before the composition is vulcanized. Non-limiting examples of plasticizers include mineral oils (paraffinic, aromatic, or naphthenic), organic esters, resins, waxes, ester plasticizers, and naturally derived oils, such as soybean oil, vegetable oil, or orange oil.

In some embodiments, a Composition of the Disclosure comprises from about 15 wt/wt % to about 85 wt/wt % of one or more plasticizers. In some embodiments, the composition comprises from about 1 wt/wt % to about 5 wt/wt %, from about 1 wt/wt % to about 15 wt/wt %, from about 1 wt/wt % to about 25 wt/wt %, from about 1 wt/wt % to about 35 wt/wt %, from about 1 wt/wt % to about 45 wt/wt %, from about 1 wt/wt % to about 55 wt/wt %, from about 1 wt/wt % to about 65 wt/wt %, from about 1 wt/wt % to about 75 wt/wt %, from about 1 wt/wt % to about 85 wt/wt %, from about 1 wt/wt % to about 95 wt/wt %, from about 5 wt/wt % to about 15 wt/wt %, from about 5 wt/wt % to about 25 wt/wt %, from about 5 wt/wt % to about 35 wt/wt %, from about 5 wt/wt % to about 45 wt/wt %, from about 5 wt/wt % to about 55 wt/wt %, from about 5 wt/wt % to about 65 wt/wt %, from about 5 wt/wt % to about 75 wt/wt %, from about 5 wt/wt % to about 85 wt/wt %, from about 5 wt/wt % to about 95 wt/wt %, from about 15 wt/wt % to about 25 wt/wt %, from about 15 wt/wt % to about 35 wt/wt %, from about 15 wt/wt % to about 45 wt/wt %, from about 15 wt/wt % to about 55 wt/wt %, from about 15 wt/wt % to about 65 wt/wt %, from about 15 wt/wt % to about 75 wt/wt %, from about 15 wt/wt % to about 95 wt/wt %, from about 25 wt/wt % to about 35 wt/wt %, from about 25 wt/wt % to about 45 wt/wt %, from about 25 wt/wt % to about 55 wt/wt %, from about 25 wt/wt % to about 65 wt/wt %, from about 25 wt/wt % to about 75 wt/wt %, from about 25 wt/wt % to about 85 wt/wt %, from about 25 wt/wt % to about 95 wt/wt %, from about 35 wt/wt % to about 45 wt/wt %, from about 35 wt/wt % to about 55 wt/wt %, from about 35 wt/wt % to about 65 wt/wt %, from about 35 wt/wt % to about 75 wt/wt %, from about 35 wt/wt % to about 85 wt/wt %, from about 35 wt/wt % to about 95 wt/wt %, from about 45 wt/wt % to about 55 wt/wt %, from about 45 wt/wt % to about 65 wt/wt %, from about 45 wt/wt % to about 75 wt/wt %, from about 45 wt/wt % to about 85 wt/wt %, from about 45 wt/wt % to about 95 wt/wt %, from about 55 wt/wt % to about 65 wt/wt %, from about 55 wt/wt % to about 75 wt/wt %, from about 55 wt/wt % to about 85 wt/wt %, from about 55 wt/wt % to about 95 wt/wt %, from about 65 wt/wt % to about 75 wt/wt %, from about 65 wt/wt % to about 85 wt/wt %, from about 65 wt/wt % to about 95 wt/wt %, from about 75 wt/wt % to about 85 wt/wt %, from about 75 wt/wt % to about 95 wt/wt %, or from about 85 wt/wt % to about 95 wt/wt % of one or more plasticizers.

In some embodiments, a Composition of the Disclosure comprises about 15 wt/wt % of one or more plasticizers. In some embodiments, the composition comprises about 1 wt/wt %, about 5 wt/wt %, about 10 wt/wt %, about 20 wt/wt %, about 25 wt/wt %, about 30 wt/wt %, about 35 wt/wt %, about 40 wt/wt %, about 45 wt/wt %, about 50 wt/wt %, about 55 wt/wt %, about 60 wt/wt %, about 65 wt/wt %, about 70 wt/wt %, about 75 wt/wt %, about 80 wt/wt %, about 85 wt/wt %, about 90 wt/wt %, or about 95 wt/wt % f one or more plasticizers.

In some embodiments, a Composition of the Disclosure comprises from about 1 phr to about 20 phr of one or more plasticizers. In some embodiments, the composition comprises from about 0.1 phr to about 1 phr, from about 0.1 phr to about 5 phr, from about 0.1 phr to about 10 phr, from about 0.1 phr to about 15 phr, from about 0.1 phr to about 20 phr, from about 0.1 phr to about 25 phr, from about 0.1 phr to about 30 phr, from about 0.1 phr to about 35 phr, from about 0.1 phr to about 40 phr, from about 1 phr to about 5 phr, from about 1 phr to about 10 phr, from about 1 phr to about 15 phr, from about 1 phr to about 25 phr, from about 1 phr to about 30 phr, from about 1 phr to about 35 phr, from about 1 phr to about 40 phr, from about 5 phr to about 10 phr, from about 5 phr to about 15 phr, from about 5 phr to about 20 phr, from about 5 phr to about 25 phr, from about 5 phr to about 30 phr, from about 5 phr to about 35 phr, from about 5 phr to about 40 phr, from about 10 phr to about 15 phr, from about 10 phr to about 20 phr, from about 10 phr to about 25 phr, from about 10 phr to about 30 phr, from about 10 phr to about 35 phr, from about 10 phr to about 40 phr, from about 15 phr to about 20 phr, from about 15 phr to about 25 phr, from about 15 phr to about 30 phr, from about 15 phr to about 35 phr, from about 15 phr to about 40 phr, from about 20 phr to about 25 phr, from about 20 phr to about 30 phr, from about 20 phr to about 35 phr, from about 20 phr to about 40 phr, from about 25 phr to about 30 phr, from about 25 phr to about 35 phr, from about 25 phr to about 40 phr, from about 30 phr to about 35 phr, from about 30 phr to about 40 phr, or from about 35 phr to about 40 phr of one or more plasticizers.

In some embodiments, a Composition of the Disclosure comprises about 10 phr of one or more plasticizers. In some embodiments, the composition comprises about 0.1 phr, about 1 phr, about 5 phr, about 15 phr, about 20 phr, about 25 phr, about 30 phr, about 35 phr, or about 40 phr of one or more plasticizers.

In some embodiments, a Composition of the Disclosure further comprises a second antidegradant that is not a Compound of the Disclosure. In some embodiments, the second antidegradant is an antioxidant. In some embodiments, the second antidegradant is an antiozonant. Non-limiting examples of antidegradants include paraphenylenediamines (PPDs), trimethyl-dihydroquinolines (TMQs), phenolics, alkylated diphenylamines (DPAs), diphenylamine-ketone condensates, and natural antidegradants. Non-limiting examples of PPDs include N¹-(4-methylpentan-2-yl)-N⁴-phenylbenzene-1,4-diamine (6PPD), N-(1,4-dimethylpentyl)-N′-phenyl-p-phenylenediamine (7PPD), N¹-phenyl-N⁴-(propan-2-yl)benzene-1,4-diamine (IPPD), N,N′-di-sec-butyl-p-phenylenediamine (44PD), N,N′-bis(1,3-dimethylbutyl)-p-phenylenediamine (66PD), N,N′-bis(1,4-dimethylpentyl)-p-phenylenediamine (77PD), and N—N′-dioctyl-p-phenylenediamine (88PD). Non-limiting examples of TMQs include 2,2,4-trimethyl-1,2-dihydroquinoline and oligomers or polymers thereof.

In some embodiments, a Composition of the Disclosure comprises from about 15 wt/wt % to about 85 wt/wt % of a second antidegradant. In some embodiments, the composition comprises from about 1 wt/wt % to about 5 wt/wt %, from about 1 wt/wt % to about 15 wt/wt %, from about 1 wt/wt % to about 25 wt/wt %, from about 1 wt/wt % to about 35 wt/wt %, from about 1 wt/wt % to about 45 wt/wt %, from about 1 wt/wt % to about 55 wt/wt %, from about 1 wt/wt % to about 65 wt/wt %, from about 1 wt/wt % to about 75 wt/wt %, from about 1 wt/wt % to about 85 wt/wt %, from about 1 wt/wt % to about 95 wt/wt %, from about 5 wt/wt % to about 15 wt/wt %, from about 5 wt/wt % to about 25 wt/wt %, from about 5 wt/wt % to about 35 wt/wt %, from about 5 wt/wt % to about 45 wt/wt %, from about 5 wt/wt % to about 55 wt/wt %, from about 5 wt/wt % to about 65 wt/wt %, from about 5 wt/wt % to about 75 wt/wt %, from about 5 wt/wt % to about 85 wt/wt %, from about 5 wt/wt % to about 95 wt/wt %, from about 15 wt/wt % to about 25 wt/wt %, from about 15 wt/wt % to about 35 wt/wt %, from about 15 wt/wt % to about 45 wt/wt %, from about 15 wt/wt % to about 55 wt/wt %, from about 15 wt/wt % to about 65 wt/wt %, from about 15 wt/wt % to about 75 wt/wt %, from about 15 wt/wt % to about 95 wt/wt %, from about 25 wt/wt % to about 35 wt/wt %, from about 25 wt/wt % to about 45 wt/wt %, from about 25 wt/wt % to about 55 wt/wt %, from about 25 wt/wt % to about 65 wt/wt %, from about 25 wt/wt % to about 75 wt/wt %, from about 25 wt/wt % to about 85 wt/wt %, from about 25 wt/wt % to about 95 wt/wt %, from about 35 wt/wt % to about 45 wt/wt %, from about 35 wt/wt % to about 55 wt/wt %, from about 35 wt/wt % to about 65 wt/wt %, from about 35 wt/wt % to about 75 wt/wt %, from about 35 wt/wt % to about 85 wt/wt %, from about 35 wt/wt % to about 95 wt/wt %, from about 45 wt/wt % to about 55 wt/wt %, from about 45 wt/wt % to about 65 wt/wt %, from about 45 wt/wt % to about 75 wt/wt %, from about 45 wt/wt % to about 85 wt/wt %, from about 45 wt/wt % to about 95 wt/wt %, from about 55 wt/wt % to about 65 wt/wt %, from about 55 wt/wt % to about 75 wt/wt %, from about 55 wt/wt % to about 85 wt/wt %, from about 55 wt/wt % to about 95 wt/wt %, from about 65 wt/wt % to about 75 wt/wt %, from about 65 wt/wt % to about 85 wt/wt %, from about 65 wt/wt % to about 95 wt/wt %, from about 75 wt/wt % to about 85 wt/wt %, from about 75 wt/wt % to about 95 wt/wt %, or from about 85 wt/wt % to about 95 wt/wt % of a second antidegradant.

In some embodiments, a Composition of the Disclosure comprises about 15 wt/wt % of a second antidegradant. In some embodiments, the composition comprises about 1 wt/wt %, about 5 wt/wt %, about 10 wt/wt %, about 20 wt/wt %, about 25 wt/wt %, about 30 wt/wt %, about 35 wt/wt %, about 40 wt/wt %, about 45 wt/wt %, about 50 wt/wt %, about 55 wt/wt %, about 60 wt/wt %, about 65 wt/wt %, about 70 wt/wt %, about 75 wt/wt %, about 80 wt/wt %, about 85 wt/wt %, about 90 wt/wt %, or about 95 wt/wt % f a second antidegradant.

In some embodiments, a Composition of the Disclosure comprises from about 1 to about 5 phr of a second antidegradant. In some embodiments, the composition comprises from about 0.001 phr to about 0.01 phr, from about 0.001 phr to about 0.1 phr, from about 0.001 phr to about 1 phr, from about 0.001 phr to about 5 phr, from about 0.001 phr to about 7.5 phr, from about 0.001 phr to about 10 phr, from about 0.01 phr to about 0.1 phr, from about 0.01 phr to about 1 phr, from about 0.01 phr to about 5 phr, from about 0.01 phr to about 7.5 phr, from about 0.01 phr to about 10 phr, from about 0.1 phr to about 1 phr, from about 0.1 phr to about 5 phr, from about 0.1 phr to about 7.5 phr, from about 0.1 phr to about 10 phr, from about 1 phr to about 7.5 phr, from about 1 phr to about 10 phr, from about 5 phr to about 7.5 phr, from about 5 phr to about 10 phr, or from about 7.5 phr to about 10 phr.

In some embodiments, a Composition of the Disclosure comprises about 3 phr of a second antidegradant. In some embodiments, the composition comprises about 0.001 phr, about 0.01 phr, about 0.1 phr, about 1 phr, about 2 phr, about 4 phr, about 5 phr, about 7.5 phr, or about 10 phr of a second antidegradant.

In some embodiments, the disclosure provides a composition comprising a Compound of the Disclosure and one or more carriers. The term “carrier” as used herein refers to a solid that can adsorb a liquid while retaining the general properties of a solid at room temperature. In some embodiments, the carrier is an inert material. In some embodiments, the carrier has a high surface area. In some embodiments, the carrier comprises particles with diameters of less than 500 microns.

In some embodiments, the composition comprises from about 15 wt/wt % to about 85 wt/wt % of one or more carriers. In some embodiments, the composition comprises from about 1 wt/wt % to about 5 wt/wt %, from about 1 wt/wt % to about 15 wt/wt %, from about 1 wt/wt % to about 25 wt/wt %, from about 1 wt/wt % to about 35 wt/wt %, from about 1 wt/wt % to about 45 wt/wt %, from about 1 wt/wt % to about 55 wt/wt %, from about 1 wt/wt % to about 65 wt/wt %, from about 1 wt/wt % to about 75 wt/wt %, from about 1 wt/wt % to about 85 wt/wt %, from about 1 wt/wt % to about 95 wt/wt %, from about 5 wt/wt % to about 15 wt/wt %, from about 5 wt/wt % to about 25 wt/wt %, from about 5 wt/wt % to about 35 wt/wt %, from about 5 wt/wt % to about 45 wt/wt %, from about 5 wt/wt % to about 55 wt/wt %, from about 5 wt/wt % to about 65 wt/wt %, from about 5 wt/wt % to about 75 wt/wt %, from about 5 wt/wt % to about 85 wt/wt %, from about 5 wt/wt % to about 95 wt/wt %, from about 15 wt/wt % to about 25 wt/wt %, from about 15 wt/wt % to about 35 wt/wt %, from about 15 wt/wt % to about 45 wt/wt %, from about 15 wt/wt % to about 55 wt/wt %, from about 15 wt/wt % to about 65 wt/wt %, from about 15 wt/wt % to about 75 wt/wt %, from about 15 wt/wt % to about 95 wt/wt %, from about 25 wt/wt % to about 35 wt/wt %, from about 25 wt/wt % to about 45 wt/wt %, from about 25 wt/wt % to about 55 wt/wt %, from about 25 wt/wt % to about 65 wt/wt %, from about 25 wt/wt % to about 75 wt/wt %, from about 25 wt/wt % to about 85 wt/wt %, from about 25 wt/wt % to about 95 wt/wt %, from about 35 wt/wt % to about 45 wt/wt %, from about 35 wt/wt % to about 55 wt/wt %, from about 35 wt/wt % to about 65 wt/wt %, from about 35 wt/wt % to about 75 wt/wt %, from about 35 wt/wt % to about 85 wt/wt %, from about 35 wt/wt % to about 95 wt/wt %, from about 45 wt/wt % to about 55 wt/wt %, from about 45 wt/wt % to about 65 wt/wt %, from about 45 wt/wt % to about 75 wt/wt %, from about 45 wt/wt % to about 85 wt/wt %, from about 45 wt/wt % to about 95 wt/wt %, from about 55 wt/wt % to about 65 wt/wt %, from about 55 wt/wt % to about 75 wt/wt %, from about 55 wt/wt % to about 85 wt/wt %, from about 55 wt/wt % to about 95 wt/wt %, from about 65 wt/wt % to about 75 wt/wt %, from about 65 wt/wt % to about 85 wt/wt %, from about 65 wt/wt % to about 95 wt/wt %, from about 75 wt/wt % to about 85 wt/wt %, from about 75 wt/wt %, to about 95 wt/wt %, or from about 85 wt/wt %, to about 95 wt/wt % of one or more carriers.

In some embodiments, the composition comprises about 15 wt/wt % of one or more carriers. In some embodiments, the composition comprises about 1 wt/wt %, about 5 wt/wt %, about 10 wt/wt %, about 20 wt/wt %, about 25 wt/wt %, about 30 wt/wt %, about 35 wt/wt %, about 40 wt/wt %, about 45 wt/wt %, about 50 wt/wt %, about 55 wt/wt %, about 60 wt/wt %, about 65 wt/wt %, about 70 wt/wt %, about 75 wt/wt %, about 80 wt/wt %, about 85 wt/wt %, about 90 wt/wt %, or about 95 wt/wt % of one or more carriers.

The present disclosure also provides processes for preparing a composition comprising a Compound of the Disclosure and one or more carriers, the process comprising admixing the compound and the one or more carriers.

The present disclosure also provides lubricant compositions comprising a Compound of the Disclosure and a lubricant. Non-limiting examples of lubricants include mineral oil, higher molecular weight petroleum distillates such as aromatic, naphthenic, and paraffinic distillates, synthetic oils such as polyalpha-olefin (PAO), synthetic esters, polyalkylene glycols (PAG), phosphate esters, perfluoropolyether (PFPE), alkylated naphthlalenes (AN), silicate esters, ionic fluids, and multiply alkylated cyclopentanes (MAC), solid lubricants such as polytetrafluoroethylene (PTFE), graphite, hexagonal boron nitride, molybdenum disulfide, tungsten disulfide, aqueous lubricants such as hydrated brush polymers, and biolubricants such as triglyceride esters, high oleic canola oil, castor oil, palm oil, sunflower seed oil, and rapeseed oil.

The present disclosure also provides combustible fuel compositions comprising a combustible fuel and a Compound of the Disclosure. Non-limiting examples of combustible fuel include gasoline, diesel, kerosene, liquefied petroleum gas, synthetic fuel, and biodisesel.

The present disclosure also provides fuel additive compositions comprising a fuel additive and a Compound of the Disclosure. Non-limiting examples of fuel additives include oxygenates such as alcohols and ethers, antioxidants, stabilizers, detergents, antiknock agents, lead scavengers, fuel dyes, viscosity modifiers, and butyl rubber. In some embodiments, the butyl rubber is in the form of polyisobutylene succinimide. In some embodiments, the butyl rubber is added as a detergent to prevent fouling of diesel fuel injectors.

### Vulcanized Elastomeric Articles

The present disclosure also provides vulcanized elastomeric articles comprising a Compound of the Disclosure. The term “vulcanized elastomeric article” refers to an article that is made by forming a composition comprising an elastomer into a specific shape and vulcanizing the composition to provide the article.

The present disclosure also provides vulcanized elastomeric articles prepared using a composition described herein.

In some embodiments, the vulcanized elastomeric article is a tire. In some embodiments, the tire is a passenger vehicle tire, a light truck tire, a heavy truck or bus tire, a motorcycle tire, an agriculture tire, an earthmover tire, an airplane tire, or a racing tire.

In some embodiments, the vulcanized elastomeric article is a component of a tire. In some embodiments, the component is a bead, a belt, a body ply, an inner liner, a sidewall, an undertread, or a tread.

In some embodiments, the vulcanized elastomeric article is a rubber overshoe, a sealing strip, an acoustic panel, an air spring, a bellow, a membrane, a tactile sensor, a crash pad, a hose, a conveyor belt, or a flooring.

### Processes

The present disclosure also provides processes for preparing a vulcanized elastomeric article, the process comprising:
(a) forming a composition described herein into a formed shape; and
(b) vulcanizing the formed shape, to provide a vulcanized elastomeric article.

In some embodiments, the vulcanizing is performed at an average temperature of from about 140° C. to about 160° C. In some embodiments, the vulcanizing is performed at an average temperature of from about 80° C. to about 100° C., from about 80° C. to about 120° C., from about 80° C. to about 140° C., from about 80° C. to about 160° C., from about 80° C. to about 180° C., from about 80° C. to about 200° C., from about 100° C. to about 120° C., from about 100° C. to about 140° C., from about 100° C. to about 160° C., from about 100° C. to about 180° C., from about 100° C. to about 200° C., from about 120° C. to about 140° C., from about 120° C. to about 160° C., from about 120° C. to about 180° C., from about 120° C. to about 200° C., from about 140° C. to about 180° C., from about 140° C. to about 200° C., from about 160° C. to about 180° C., from about 160° C. to about 200° C., or from about 180° C. to about 200° C.

In some embodiments, the vulcanizing is performed at an average temperature of about 150° C. In some embodiments, the vulcanizing is performed at an average temperature of about 80° C., about 100° C., about 120° C., about 140° C., about 160° C., about 180° C., or about 200° C.

The present disclosure also provides processes for retreading tires, the process comprising:
(a) applying a composition described herein to a tire;
(b) disposing a pre-vulcanized tread around the tire;
(c) disposing a curing envelope around the tire; and
(d) vulcanizing the tire.

### Kits

The present disclosure also provides kits comprising a composition described herein, packaged in a manner, e.g., in a container, that facilitates use of the composition to practice the processes and/or methods of the present disclosure. In some embodiments, the kit comprises a composition described herein and instructions for using the composition in a vulcanizable elastomeric composition. In some embodiments, the kit comprises a composition described herein and instructions for using the composition to prepare a vulcanized elastomeric article. The composition may be packaged in any suitable container, such as a sealed bottle or vessel, with a label affixed to the container or included in the kit that describes the composition and proper use thereof.

The disclosure also provides the following specific embodiments.

Embodiment I. A compound having Formula (I):

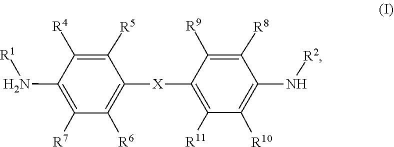

or a salt or solvate thereof, wherein:
R¹ is selected from the group consisting of C₁-C₁₂ alkyl, —CHR^{1a}R^{1b}, C₃-C₆ cycloalkyl, and optionally substituted phenyl; or
R¹ and R⁴ taken together with the nitrogen and carbon atoms, respectively, to which they are attached form a saturated or partially saturated optionally substituted 5- or 6-membered heterocyclo;
R^{1a} is selected from the group consisting of optionally substituted phenyl, C₃-C₆ cycloalkyl, and optionally substituted 5- or 6-membered heteroaryl;
R^{1b} is selected from the group consisting of hydrogen and C₁-C₆ alkyl;
R² is selected from the group consisting of C₁-C₁₂ alkyl, —CHR^{2a}R^{2b}, C₃-C₆ cycloalkyl, and optionally substituted phenyl; or
R² and R⁸ taken together with the nitrogen and carbon atoms, respectively, to which they are attached form a saturated or partially saturated optionally substituted 5- or 6-membered heterocyclo;
R^{2a} is selected from the group consisting of optionally substituted phenyl, C₃-C₆ cycloalkyl, and optionally substituted 5- or 6-membered heteroaryl;
R^{2b} is selected from the group consisting of hydrogen and C₁-C₆ alkyl;
X is selected from the group consisting of —CHR^{3a}—, —NR^{3b}—, —O—, and —S—;
R^{3a} is selected from the group consisting of C₂-C₁₂ alkyl, C₂-C₆ alkenyl, C₃-C₆ cycloalkyl, optionally substituted phenyl, optionally substituted 5- or 6-membered heterocyclo, and optionally substituted 5- or 6-membered heteroaryl;
R^{3b} is selected from the group consisting of hydrogen, C₁-C₁₂ alkyl, C₃-C₆ cycloalkyl, optionally substituted phenyl, optionally substituted 5- or 6-membered heterocyclo, and optionally substituted 5- or 6-membered heteroaryl;
R⁴, R⁵, R⁶, and R⁷ are independently selected from the group consisting of hydrogen, halogen, C₁-C₆ alkyl, C₁-C₆ alkoxy, and C₁-C₆ alkylthio; or
R¹ and R⁴ taken together with the nitrogen and carbon atoms, respectively, to which they are attached form a saturated or partially saturated optionally substituted 5- or 6-membered heterocyclo; and
R⁵, R⁶, and R⁷ are independently selected from the group consisting of hydrogen, halogen, C₁-C₆ alkyl, C₁-C₆ alkoxy, and C₁-C₆ alkylthio; and
R⁸, R⁹, R¹⁰, and R¹¹ are independently selected from the group consisting of hydrogen, halogen, C₁-C₆ alkyl, C₁-C₆ alkoxy, and C₁-C₆ alkylthio; or
R² and R⁸ taken together with the nitrogen and carbon atoms, respectively, to which they are attached form a saturated or partially saturated optionally substituted 5- or 6-membered heterocyclo; and
R⁹, R¹⁰, and R¹¹ are independently selected from the group consisting of hydrogen, halogen, C₁-C₆ alkyl, C₁-C₆ alkoxy, and C₁-C₆ alkylthio, with the provisos that (i) if X is O— or —S—, then R¹ is selected from the group consisting of methyl, ethyl, pentyl, hexyl, heptyl, nonyl, decyl, undecyl, dodecyl, —CHR^{1a}R^{1b}, C₃-C₆ cycloalkyl, and optionally substituted phenyl; or
R¹ and R⁴ taken together with the nitrogen and carbon atoms, respectively, to which they are attached form a saturated or partially saturated optionally substituted 5- or 6-membered heterocyclo; and
R² is selected from the group consisting of methyl, ethyl, pentyl, hexyl, heptyl, nonyl, decyl, undecyl, dodecyl, —CHR^{2a}R^{2b}, C₃-C₆ cycloalkyl, and optionally substituted phenyl; or
R² and R⁸ taken together with the nitrogen and carbon atoms, respectively, to which they are attached form a saturated or partially saturated optionally substituted 5- or 6-membered heterocyclo; and (ii) if X is —CHCH₃—, then R¹ is selected from the group consisting of methyl, ethyl, propyl, butyl, tert-butyl, pentyl, hexyl, heptyl, octyl, nonyl, decyl, undecyl, dodecyl, —CHR^{1a}R^{1b}, C₃-C₆ cycloalkyl, and optionally substituted phenyl; or
R¹ and R⁴ taken together with the nitrogen and carbon atoms, respectively, to which they are attached form a saturated or partially saturated optionally substituted 5- or 6-membered heterocyclo; and
R² is selected from the group consisting of methyl, ethyl, propyl, butyl, tert-butyl, pentyl, hexyl, heptyl, octyl, nonyl, decyl, undecyl, dodecyl, —CHR^{2a}R^{2b}, C₃-C₆ cycloalkyl, and optionally substituted phenyl; or
R² and R⁸ taken together with the nitrogen and carbon atoms, respectively, to which they are attached form a saturated or partially saturated optionally substituted 5- or 6-membered heterocyclo.

Embodiment II. The compound of Embodiment I, wherein X is —CHR^{3a}—.

Embodiment III. The compound of Embodiment I or II, wherein R^{3a} is selected from the group consisting of optionally substituted phenyl, optionally substituted 5- or 6-membered heterocyclo, and optionally substituted 5- or 6-membered heteroaryl.

Embodiment IV. The compound of Embodiment III, wherein R^{3a} is:

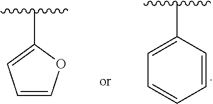 [SMILES: *c1ccco1 ; *c1ccccc1]

Embodiment V. The compound of Embodiment I, wherein X is —NH—.

Embodiment VI. The compound of Embodiment I, wherein X is —O—.

Embodiment VII. The compound of Embodiment I, wherein X is —S—.

Embodiment VIII. The compound of any one of Embodiments I-VII, wherein R¹ is C₁-C₁₂ alkyl or —CHR^{1a}R^{1b} and R² is C₁-C₁₂ alkyl or —CHR^{2a}R^{2b}.

Embodiment IX. The compound of Embodiment VIII, wherein R^{1a} and R^{2a} are:

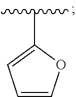 [SMILES: *c1ccco1]

and
R^{1b} and R² are hydrogen.

Embodiment X. The compound of Embodiment VIII, wherein R¹ and R² are isopropyl.

Embodiment XI. A composition comprising:
(i) the compound of any one of Embodiments I-X; and
(ii) one or more elastomers; or
(iii) one or more fillers; or
(iv) one or more rubber chemicals; or
(v) one or more plasticizers; or
(vi) a second antidegradant; or
(vii) a combination of one or more elastomers, one or more fillers, one or more rubber chemicals, one or more plasticizers, and/or a second antidegradant.

### EXAMPLES

### Example 1

### Synthesis of bis(2,2,4-trimethyl-1,2-dihydroquinolin-6-yl)methane (Compound 1)

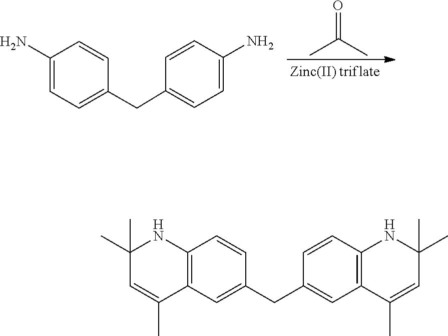

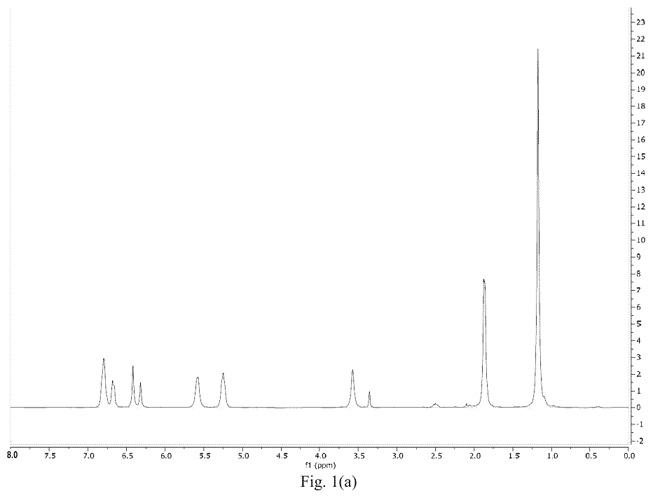

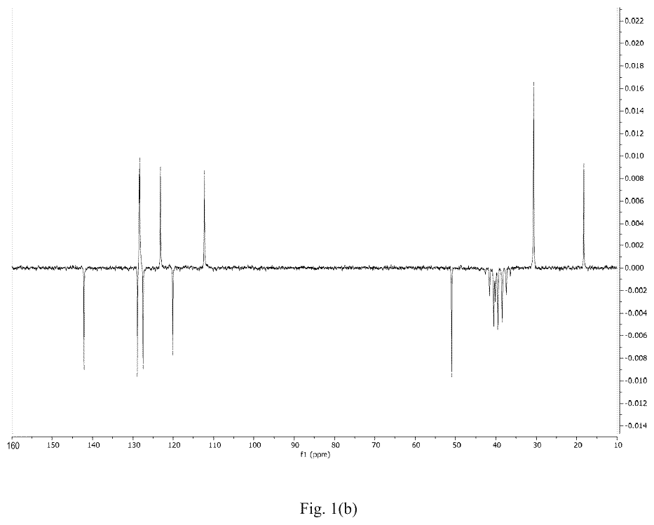

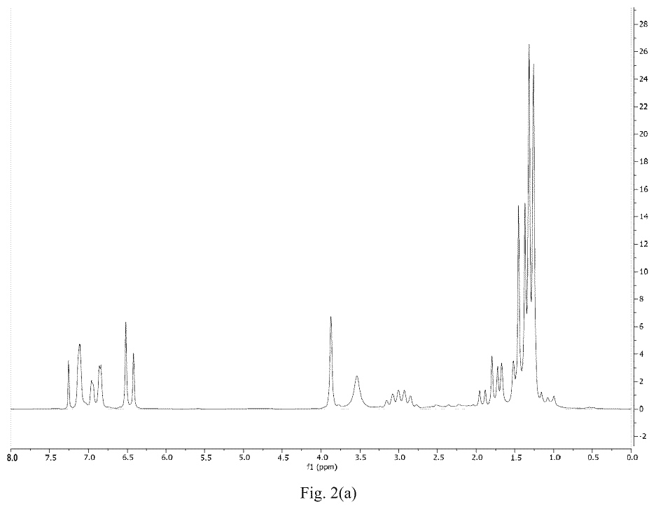

To a 100-mL flask fitted with a stir-bar and a thermometer were loaded 4,4′-methylenedianiline (0.918 g; 4.63 mmol) and acetone (3.5 mL). The mixture was stirred under N₂ protection. Zinc(II) triflate (0.1755 g; 4.83 mmol) was loaded in one portion. The reaction mixture was stirred at room temperature for 6-7 days. The obtained dark slurry was loaded into a chromatography column (2.4 cm inner diameter) previously prepared with silica gel 60 (70-230 mesh) and eluent (20% ethyl acetate in hexanes). The fractions at R_{f}=0.8 were collected and stripped of volatiles under reduced pressure (rotary evaporator; water bath=50° C.). The obtained solid was dried on the rotary evaporator (50° C.; <5 mbars) for 1 hour. The product was a slightly yellow solid weighing 0.41 g (25% yield). Representative ¹H and ¹³C NMR spectra are shown in FIGS. 1(a) and 1(b).

### Example 2

### Synthesis of bis(2,2,4-trimethyl-1,2,3,4-tetrahydroquinolin-6-yl)methane (Compound 2)

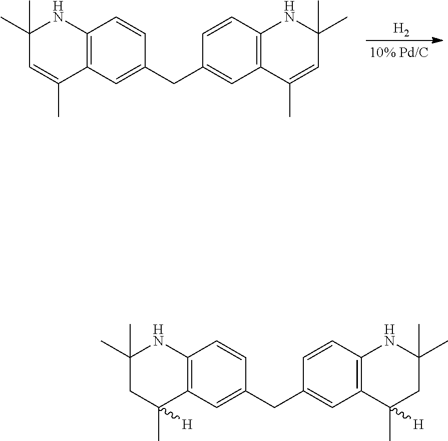

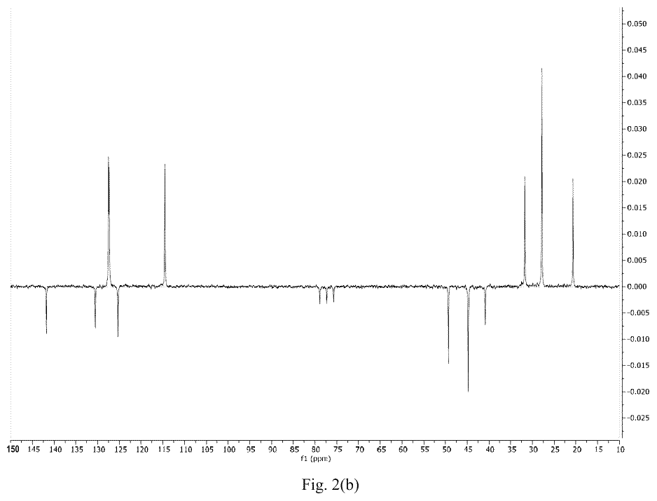

A 300 mL Paar autoclave was charged with crude Compound 1 (1.0 g; <2.79 mmol), methanol (77 mL), aqueous 37% HCl (0.46 mL; 5.58 mmol), and wet 10% Pd/C (1.0 g). The autoclave was sealed, then purged with 20 PSI nitrogen gas. The vessel was charged with 50 PSI H₂, and the reaction mixture was stirred for 1 hour at 25° C. After completion of the reaction, the reaction contents were filtered through a 0.5 m frit to separate catalyst residues from the product. The filtrate was stripped of volatiles under reduced pressure (rotary evaporator; water bath=50° C.). The crude hydrogenated material as the dihydrochloride salt was dissolved in chloroform. The emerald-green solution was transferred to a separatory funnel, washed with saturated aqueous NaHCO₃, dried over anhydrous MgSO₄, filtered, and stripped of volatiles under reduced pressure (rotary evaporator; water bath=50° C.). Further drying until constant weight provided the desired product as a green-yellow viscous liquid. Representative ¹H and ¹³C NMR spectra are shown in FIGS. 2(a) and 2(b).

### Example 3

### Synthesis of 4,4′-(furan-2-ylmethylene)bis(N-isopropylaniline) (Compound 3)

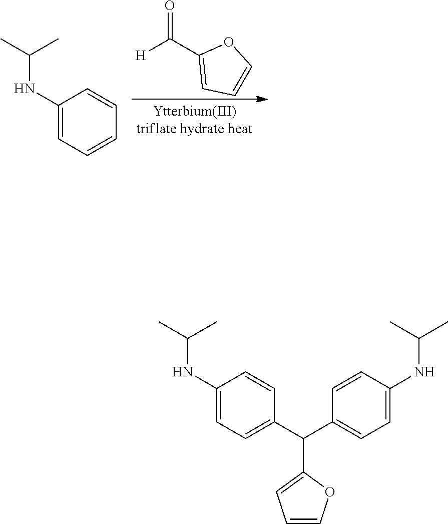

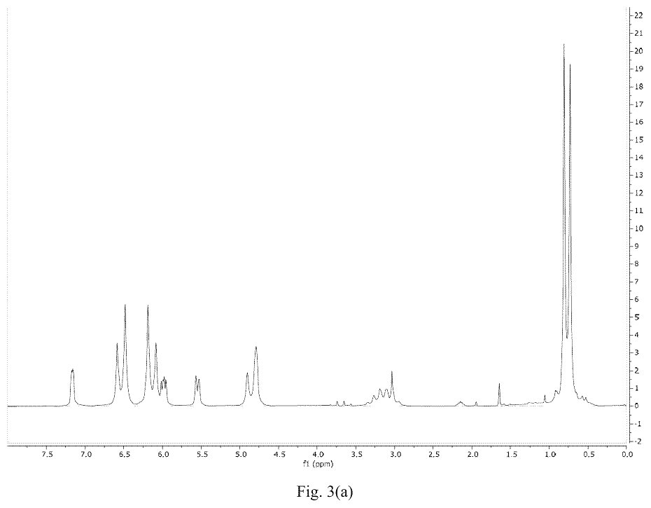

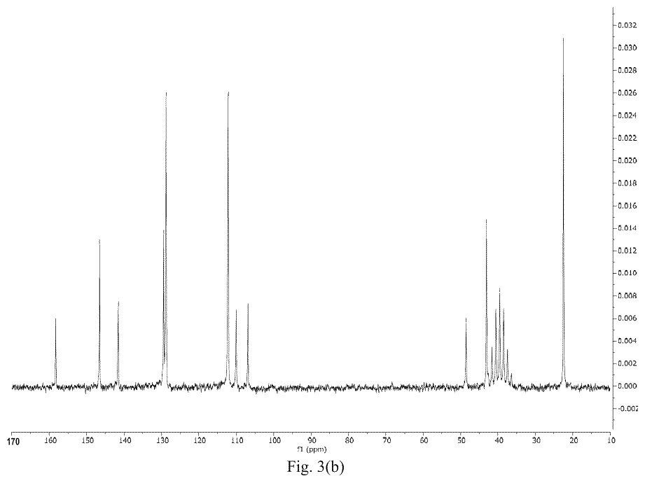

A 25-mL round bottom flask fitted with a stir-bar and a reflux condenser was loaded with N-isopropylaniline (7.0 mL; 48.7 mmol), furfural (1.6 mL; 19.3 mmol), and ytterbium(III) triflate hydrate (1.15 g; <1.85 mmol Yb³⁺). The reaction mixture was stirred under N₂ protection and heated (oil bath temperature=95° C.) for 17 hours. Upon cooling to room temperature, the obtained dark mass was dissolved in ethyl acetate (approx. 100 mL). The resulting dark solution was washed with aq. 0.5 M NaOH (20 mL), aq. 1 M NaHCO₃ (20 mL), distilled water (20 mL), and aqueous saturated NaCl (20 mL). The organic phase was stripped of most of the volatiles. The obtained dark solution was loaded to a 41 mm inner diameter column chromatography previously prepared with silica gel 60 (15-16 cm height) and eluent (10% ethyl acetate in hexanes). The column was eluted with 10% AcOEt in hexanes. Several portions (80 mL each) were collected. Each portion was characterized by TLC (10% AcOEt in hexanes). The portions containing the dot at R_{f}=0.6 (excess N-isopropylaniline), 0.4 (unknown impurity; traces), and 0.5 (unknown impurity; traces) were discarded. The portions containing the dot at R_{f}=0.3 (Compound 3) were collected, stripped of volatiles under reduced pressure (rotary evaporator; water bath=60° C.). The resulting light-brown viscous liquid was dried on the rotary evaporator (60° C.; <5 mbars) for 10 min. Yield=1.4 grams (21% yield based on furfural) as a very viscous brown liquid. Representative ¹H and ¹³C NMR spectra are shown in FIGS. 3(a) and 3(b).

### Example 4

### Synthesis of 4,4′-(furan-2-ylmethylene)bis(N-(furan-2-ylmethyl)aniline) (Compound 7)

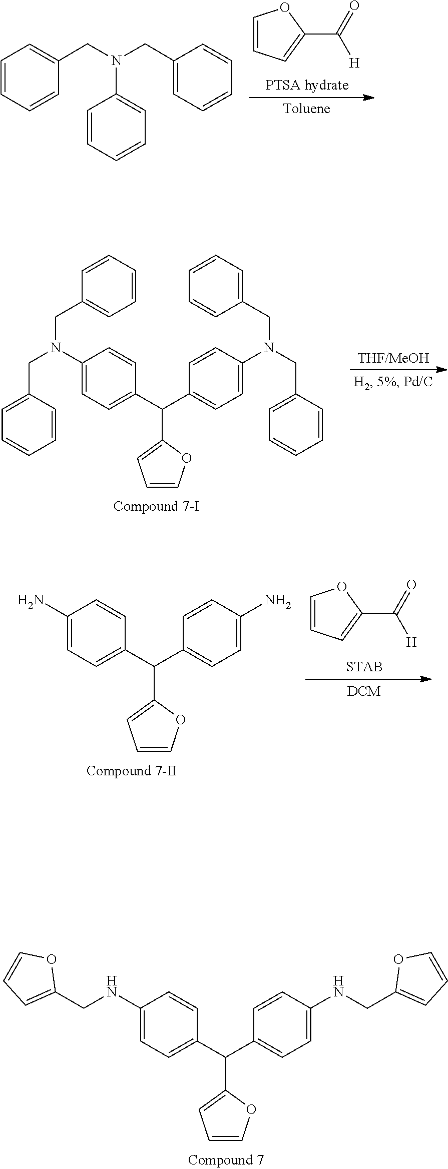

A solution of N,N-dibenzylaniline (42.58 g, 156.120 mmol, 2.14 equiv), furfural (7.00 g, 72.9 mmol, 1.0 equiv) and p-toluenesulfonic acid monohydrate (9.89 g, 51.982 mmol, 0.7 equiv) in toluene (100 mL) was heated overnight at 110° C. The reaction was cooled to room temperature and diluted with dichloromethane (300 mL). The mixture was washed with saturated sodium bicarbonate (100 mL) and the organic layer was concentrated under reduced pressure. The residue was dry loaded onto silica (70 g) and purified on an Interchim automated chromatography system (2×350 g stacked pre-packed 20 mm silica column), eluting with a gradient of 0 to 20% ethyl acetate in heptane to give intermediate compound 7-I (19.9 g, 44% yield) as a white solid.

A suspension of compound 7-I (47.0 g, 75.22 mmol, 1.0 equiv) and 5% palladium on carbon (2.35 g, 50% water wet) in a 1:1 mixture of methanol (235 mL) and THE (235 mL, 5 vol) was hydrogenated @25 psi for 2 hours. Upon completion, the reaction was filtered through Celite (30 g), which was washed with methanol (200 mL). The filtrate was concentrated under reduced pressure. The residue was dry loaded onto silica gel (50 g) and purified on an Interchim automated chromatography system (220 g, pre-packed 60 mm silica column), eluting with a gradient of 25 to 100% ethyl acetate in heptane to give compound 7-II (13.7 g, 69% yield) as an off white solid and 4,4′-((tetrahydrofuran-2-yl)methylene)dianiline (2.1 g, 10% yield) as an off white solid.

A solution of compound 7-II (13.7 g, 51.83 mmol, 1.0 equiv) and furfural (9.96 g, 103.66 mmol, 2.0 equiv) in dichloromethane (360 mL, 36 vol) was stirred for 1 hour. Sodium triacetoxyborohydride (30.76 g, 145.123 mmol, 2.8 equiv) was added at room temperature and the reaction was stirred for one hour. LCMS and NMR analysis of an indicated completion of the reaction. The reaction was quenched with saturated sodium bicarbonate (360 mL) and stirred for 1 hour. The layers were separated, and the aqueous layer was extracted with dichloromethane (2×130 mL). The combined organics were washed with saturated brine (130 mL) and concentrated under reduced pressure. The residue was then dry loaded onto silica (40 g) and purified on an Interchim automated chromatography system (250 g, pre-packed 60 μm silica column), eluting with a gradient of 0 to 30% ethyl acetate in heptane. The fractions containing pure product were then concentrated under reduced pressure and dried under high vacuum at 50° C. overnight to give compound 7 (18.3 g, 83% yield, >95% HPLC purity) as an off-white oil. ¹H NMR (400 MHz, DMSO-d₆) δ=7.58-7.51 (m, 3H), 6.86 (d, J=8.6 Hz, 4H), 6.66-6.50 (m, 4H), 6.40-6.33 (m, 3H), 6.28 (dd, J=0.7, 3.2 Hz, 2H), 6.01-5.86 (m, 3H), 5.19-5.12 (m, 1H), 4.20 (d, J=6.0 Hz, 4H); ¹³C (100 MHz, DMSO-d₆) δ=158.52, 153.93, 147.23, 142.31, 142.19, 130.74, 129.16, 112.61, 110.81, 110.58, 107.42, 107.27, 48.94; MS (ESI) m/z [M+H]⁺ calcd. for C₂₇H₂₄N₂O₃, 425.2, found, 425.2.

### Example 5

### Synthesis of 4,4′-methylenebis(N-(furan-2-ylmethyl)aniline) (Compound 8)

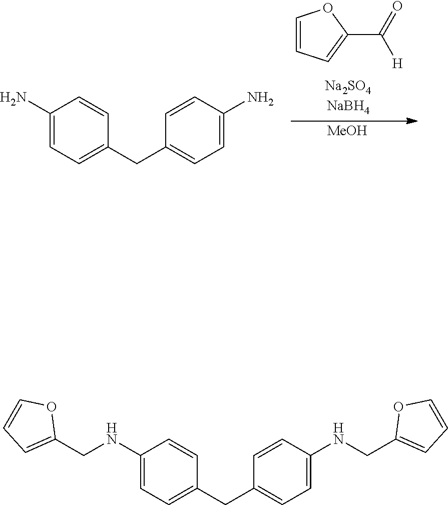 [SMILES: "MeOH" ; O=Cc1ccco1 ; Nc1ccc(Cc2ccc(N)cc2)cc1 ; c1coc(CNc2ccc(Cc3ccc(NCc4ccco4)cc3)cc2)c1]

Furfural (8.80 mL, 106 mmol, 2.1 equiv) and sodium sulfate (14.3 g, 101 mmol, 2.0 equiv) were added to a solution of 4,4′-methylenedianiline (10.0 g, 50.4 mmol, 1.0 equiv) in methanol (100 mL) in a 3-neck 500 mL round bottom flask. The reaction was stirred at room temperature for 1 hour. Sodium borohydride (4.00 g, 101 mmol, 2.1 equiv) was added portion wise over 15 minutes (caution exotherm, methanol began to reflux). The reaction was vigorously stirred at room temperature for 1 hour. The mixture was transferred to a 1-neck round bottom with methanol and concentrated under reduced pressure. The residue was quenched with water (200 mL) then diluted with ethyl acetate (400 mL). The layers were separated and the aqueous was extracted with ethyl acetate (2×200 mL). The combined organics were dried over sodium sulfate, filtered and concentrated under reduced pressure. The residue was absorbed onto silica gel (40 g) using dichloromethane (loading with ethyl acetate resulted in solubility issues during purification) and purified on a Biotage automated chromatography system (350 g, Biotage 60 μm silica gel column), eluting with a gradient of 0 to 30% ethyl acetate in hexanes. The resulting solid was dried under vacuum at room temperature for 18 hours to give compound 8 (13.9 g, 77% yield, >95% HPLC purity) as an off-white solid. ¹H NMR (400 MHz, CDCl₃) δ=7.34 (dd, J=0.8, 1.8 Hz, 2H), 7.00-6.96 (m, 4H), 6.60-6.56 (m, 4H), 6.30 (dd, J=1.8, 3.2 Hz, 2H), 6.20 (dd, J=0.7, 3.2 Hz, 2H), 4.26 (s, 4H), 3.89 (br s, 2H), 3.76 (s, 2H); ¹³C NMR (101 MHz, CDCl₃) d=152.97 (s, 2C), 145.81, 141.91 (s, 2C), 131.54 (s, 2C), 129.62 (s, 2C), 113.33 (s, 4C), 110.36 (s, 2C), 106.95 (s, 2C), 41.74 (s, 2C), 40.17; MS (ESI) m/z [M+H]⁺ calcd. for C₂₃H₂₂N₂O₂, 359.2, found, 359.1.

### Example 6

### Synthesis of 4,4′-(phenylmethylene)bis(N-isopropylaniline) (Compound 16)

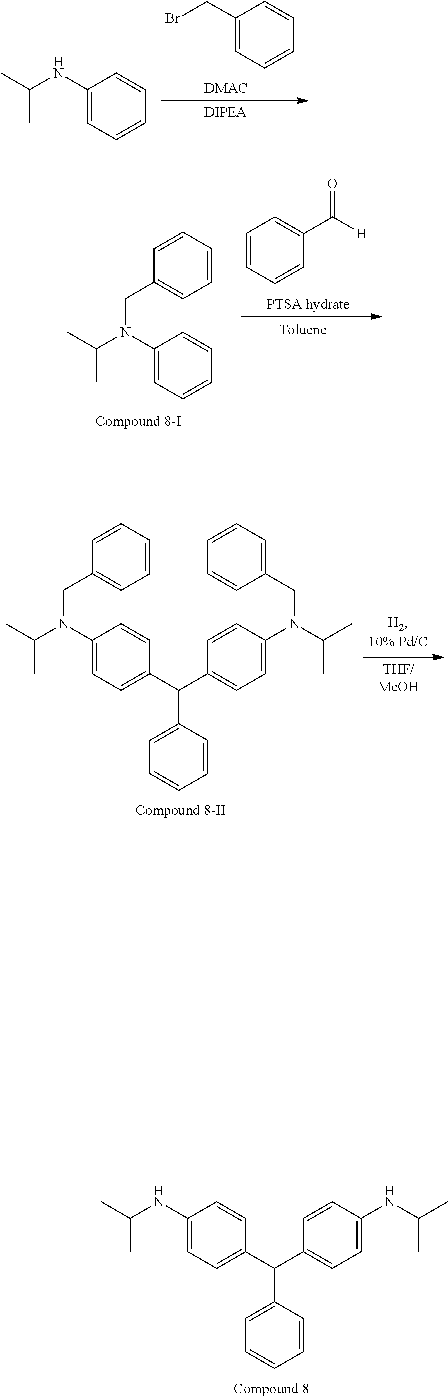

N,N-Diisopropylethylamine (155 mL, 888 mmol, 1.5 equiv) was added to a solution of N-isopropylaniline (80 g, 592 mmol, 1 equiv) and benzyl bromide (121.5 g, 711 mmol, 1.2 equiv) in N,N-dimethylacetamide (700 mL). The mixture was stirred at 110° C. for 2 hours at which point LC/MS analysis indicated the reaction was complete. After cooling to room temperature, the mixture was diluted with saturated brine (1.5 L) and extracted with ethyl acetate (3×1 L). The combined organic layers were washed with saturated brine (1 L), dried over anhydrous sodium sulfate, filtered and concentrated under reduced pressure. The residue was purified over silica gel (1.2 kg), eluting with a gradient of 5 to 10% dichloromethane in heptane to give compound 8-I (114 g, 86% yield, >94% HPLC purity) as a colorless oil.

p-Toluenesulfonic acid monohydrate (32.6 g, 171.4 mmol, 0.6 equiv) was added to a solution of compound 8-I (64 g, 285.7 mmol, 1 equiv) and benzaldehyde (18.2 g, 171.4 mmol, 0.6 equiv) in toluene (500 mL). The reaction mixture was refluxed, equipped with Dean-Stark apparatus, for 24 hours at which point LC/MS analysis indicated 65% f conversion of compound 8-I to compound 8-II. After cooling to room temperature, most of the toluene was removed under reduced pressure. The residue was diluted with ethyl acetate (1.5 L). The resulting mixture was washed sequentially with saturated sodium bicarbonate (2×1 L) and saturated brine (2×1 L). The organic layer was dried over sodium sulfate, filtered and concentrated under reduced pressure to remove three fourths of the solvent. The residual solution was added to methanol (1.5 L), with vigorous stirring, over 10 minutes. The resulting precipitate was filtered, then triturated with a 10:1 mixture of methanol and ethyl acetate (3×300 mL) for 2 hours to give compound 8-II (25 g, 32% yield, >95% HPLC purity) as an off-white solid.

Compound 8-II (25 g, 70 mmol, 1 equiv) and 10% palladium on carbon (2.5 g, 50% water wet) was hydrogenated @30 psi in a 10:1 mixture of tetrahydrofuran and methanol (300 mL) at room temperature for 2.5 hours, at which point LC/MS analysis indicated the reaction was complete. The reaction was filtered through Celite (20 g), which was washed with tetrahydrofuran (200 mL). The filtrate was concentrated under reduced pressure. The residue was purified over silica gel (800 g), eluting with a gradient of 5 to 15% ethyl acetate in heptane to give compound 8 (12 g, 72% yield, >95% HPLC purity) as a colorless oil, which slowly solidified as a white solid over 2 days. ¹H NMR (400 MHz, CDCl₃) δ=7.34-7.08 (m, 5H), 6.92-6.86 (m, 4H), 6.51-6.46 (m, 4H), 5.32-5.27 (m, 1H), 3.57 (spt, J=6.3 Hz, 2H), 3.35 (s, 2H), 1.18 (d, J=6.4 Hz, 12H); ¹³C NMR (100 MHz, CDCl₃) δ=145.70, 145.56, 133.25, 130.19, 129.42, 128.05, 125.77, 113.01, 55.21, 44.37, 23.15; MS (ESI) m/z [M+H]⁺ calcd. for C₂₅H₃₀N₂, 359.2, found, 359.2.

### Example 7

### Synthesis of 4,4′-methylenebis(N-isopropylaniline) (Compound 19)

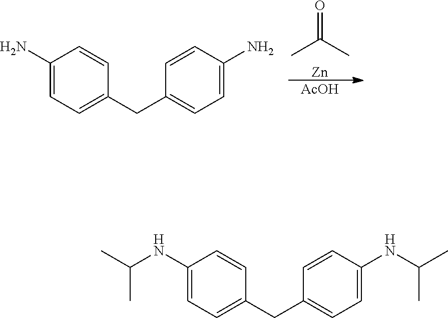

Acetic acid (150 mL) was added slowly to a mixture of 4,4′-methylenedianiline (15 g, 75.7 mmol, 1.0 equiv) and acetone (19.6 mL, 265 mmol, 3.5 equiv) at room temperature (slight exotherm). Zinc (37.5 g, 576.9 mmol, 7.6 equiv) was added in portions at room temperature. After heating at 40° C. for 2 hours, the reaction was cooled to room temperature and filtered through a plug of Celite and silica gel to remove zinc. The column was washed with water (4×100 mL) and dichloromethane (3×100 mL). The filtrate was evaporated under reduced pressure to remove acetic acid. The residue was diluted with a mixture of water (150 mL) and dichloromethane (200 mL). The pH was adjusted to 10 with 20% aqueous sodium hydroxide at 0° C. A white precipitate was formed which was further diluted with water (500 mL) to fully dissolve. The layers were separated and the aqueous layer was extracted with dichloromethane (2×100 mL). The combined organic layers were washed with saturated brine (200 mL), dried over magnesium sulfate, filtered and concentrated under reduced pressure. The residue was purified on a Biotage automated chromatography system (350 g Biotage, 60 μm silica gel column), eluting with a gradient of 0 to 10% ethyl acetate in hexanes to give compound 19 (20 g, 93% yield, 97.3% purity) as a light-yellow liquid. ¹H NMR (400 MHz, CDCl₃) δ=7.03-6.99 (m, 4H), 6.57-6.53 (m, 4H), 3.79 (s, 2H), 3.62 (spt, J=6.3 Hz, 2H), 3.35 (br s, 2H), 1.22 (d, J=6.2 Hz, 12H); ¹³C NMR (101 MHz, CDCl₃) δ=145.65 (s, 2C), 130.61 (s, 2C), 129.66 (s, 4C), 113.42 (s, 4C), 44.45 (s, 2C), 40.11, 23.14 (s, 4C); MS (ESI) m/z [M+H]⁺ calcd. for C₁₉H₂₆N₂, 283.2, found, 283.2.

### Example 8

### Synthesis of 4,4′-methylenebis(N-(1-(furan-2-yl)ethyl)aniline) (Compound 20)

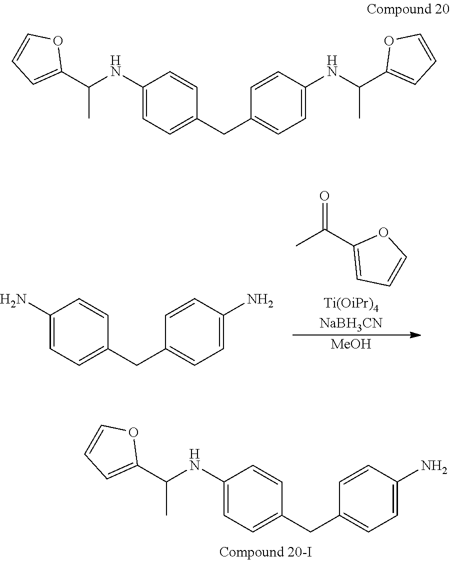

4,4′-Methylenedianiline (19.8 g, 100 mmol, 1 equiv) was added to a solution of 2-furyl methyl ketone (27.5 g, 250 mmol, 2.5 equiv) and titanium(IV) isopropoxide (28.4 g, 100 mmol, 1 equiv) in methanol (200 mL), followed by addition of sodium cyanoborohydride (18.9 g, 300 mmol, 3 equiv). The mixture was refluxed for three days. After cooling to room temperature, sodium borohydride (˜1.6 g) was added portion wise until no 2-furyl methyl ketone 2 remained by TLC analysis. The mixture was diluted with water (200 mL) and ethyl acetate (100 mL), filtered through Celite (150 g), which was rinsed with ethyl acetate (300 mL). The layers were separated and the aqueous layer was extracted with ethyl acetate (3×200 mL). The combined organic layers were dried over sodium sulfate, filtered and concentrated under reduced pressure. The residue was purified over silica gel (800 g), eluting with a gradient of 10 to 40% ethyl acetate in heptane to give compound 20 (9.17 g, 24% yield) as a light-yellow oil and compound 20-I (10.57 g, 36% yield) as a dark brown oil.

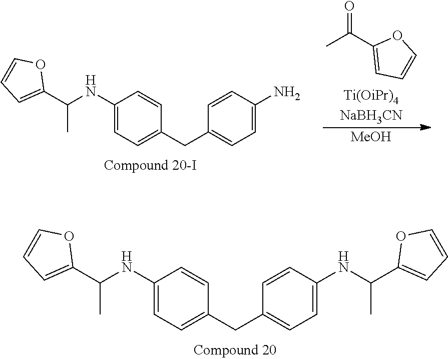

Compound 20-I (29.2 g, 100 mmol, 1 equiv) was added to a solution of 2-furyl methyl ketone 2 (22.0 g, 200 mmol, 2.0 equiv) and titanium(IV) isopropoxide (28.4 g, 100 mmol, 1 equiv) in methanol (300 mL), followed by addition of sodium cyanoborohydride (18.9 g, 300 mmol, 3 equiv). The mixture was refluxed for three days. After cooling to room temperature, sodium borohydride (˜1.6 g) was added portion wise until no 2-furyl methyl ketone remained by TLC analysis. The mixture was diluted with water (200 mL) and ethyl acetate (100 mL), filtered through Celite (150 g), which was rinsed with ethyl acetate (300 mL). The layers were separated, and the aqueous layer was extracted with ethyl acetate (3×20 mL). The combined organic layers were dried over sodium sulfate, filtered and concentrated under reduced pressure. The residue was purified over silica gel (750 g), eluting with 10% ethyl acetate in heptane to give compound 20 (19 g, 49% yield) as light-yellow oil. ¹H NMR (400 MHz, CDCl₃) δ=7.35 (dd, J=0.9, 1.8 Hz, 1H), 7.31 (dd, J=0.9, 1.8 Hz, 2H), 6.97-6.92 (m, 4H), 6.55-6.47 (m, 4H), 6.32-6.29 (m, 1H), 6.26 (dd, J=1.8, 3.2 Hz, 2H), 6.14-6.10 (m, 2H), 4.58 (q, J=6.6 Hz, 2H), 3.72 (s, 2H), 1.51 (d, J=6.7 Hz, 6H), 1.37 (dd, J=6.7, 17.4 Hz, 1H); ¹³C NMR (100 MHz, CDCl₃) δ=157.47, 145.13, 141.42, 131.27, 129.56, 113.61, 110.10, 109.99-109.79 (m, 1C), 105.61, 105.04, 49.00, 48.44, 47.60, 40.13, 21.35, 20.96, 20.11; MS (ESI) m/z [M+H]⁺ calcd. for C₂₅H₂₆N₂O₂, 387.2, found, 387.2.

### Example 9

### Synthesis of 4.4′-methylenebis(N-(1-phenylethyl)aniline) (Compound 21)

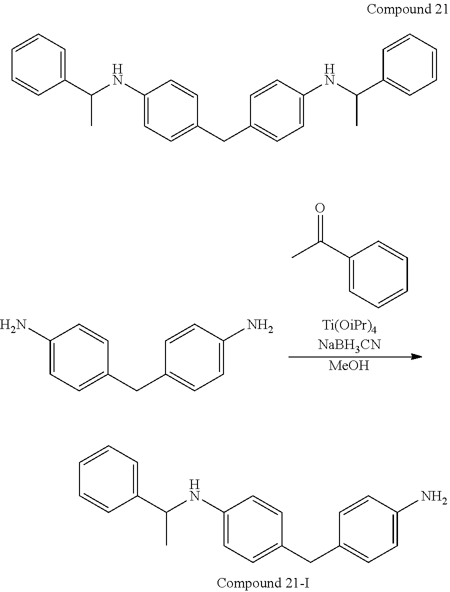

4,4′-Methylenedianiline (24.75 g, 125 mmol, 1 equiv) was added to a solution of acetophenone (37.5 g, 312.5 mmol, 2.5 equiv) and titanium(IV) isopropoxide (35.5 g, 125 mmol, 1 equiv) in methanol (250 mL), followed by addition of sodium cyanoborohydride (23.6 g, 375 mmol, 3 equiv). The mixture was refluxed for 24 hours. After cooling to room temperature, sodium borohydride (˜1.7 g) was added portion wise until no acetophenone remained by TLC analysis. The mixture was diluted with water (300 mL) and ethyl acetate (150 mL), filtered through Celite (150 g), which was rinsed with ethyl acetate (300 mL). The layers were separated and the aqueous layer was extracted with ethyl acetate (4×200 mL). The combined organic layers were dried over sodium sulfate, filtered and concentrated under reduced pressure. The residue was purified over silica gel (750 g), eluting with a gradient of 10 to 40% ethyl acetate in heptane to give compound 21 (12.6 g, 21% yield) as white solid and compound 21-I (19.6 g, 43% yield) as dark brown oil.

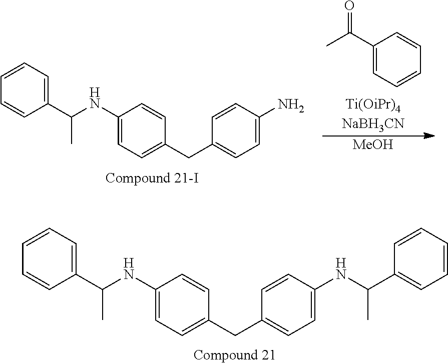

Compound 21-I (35.0 g, 112 mmol, 1 equiv) was added to a solution of acetophenone (26.9 g, 224 mmol, 2.0 equiv) and titanium(IV) isopropoxide (31.8 g, 112 mmol, 1 equiv) in methanol (225 mL), followed by addition of sodium cyanoborohydride (17.6 g, 280 mmol, 2.5 equiv). The mixture was refluxed for three days. After cooling to room temperature, sodium borohydride (˜1.7 g) was added portion wise until no acetophenone remained by TLC analysis. The mixture was diluted with water (200 mL) and ethyl acetate (200 mL), filtered through Celite (150 g), which was rinsed with ethyl acetate (300 mL). The layers were separated and the aqueous layer was extracted with ethyl acetate (3×200 mL). The combined organic layers were dried over sodium sulfate, filtered and concentrated under reduced pressure. The residue was purified over silica gel (800 g), eluting with 10% ethyl acetate in heptane to give compound 21 (22.9 g, 50% yield) as white solid. ¹H NMR (400 MHz, CDCl₃) δ=7.37-7.27 (m, 8H), 7.24-7.18 (m, 2H), 6.95-6.80 (m, 4H), 4.43 (q, J=6.7 Hz, 2H), 3.90 (br s, 2H), 3.66 (s, 2H), 1.49 (s, 3H), 1.48 (s, 3H); ¹³C NMR (100 MHz, CDCl₃) δ 145.53, 145.47, 130.77, 129.47, 128.67, 126.87, 125.93, 113.41, 60.47, 53.74, 40.12, 25.11, 14.29; MS (ESI) m/z [M+H]⁺ calcd. for C₂₉H₃NN₂, 407.2, found, 407.3.

### Example 10

### Synthesis of bis(2,2,4-trimethyl-1,2-dihydroquinolin-6-yl)sulfane (Compound 153)

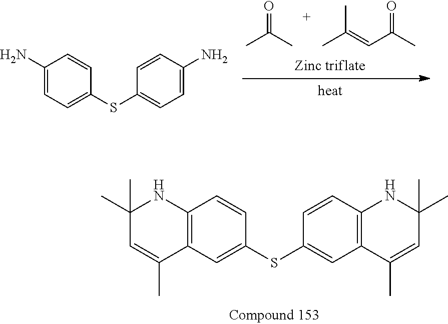

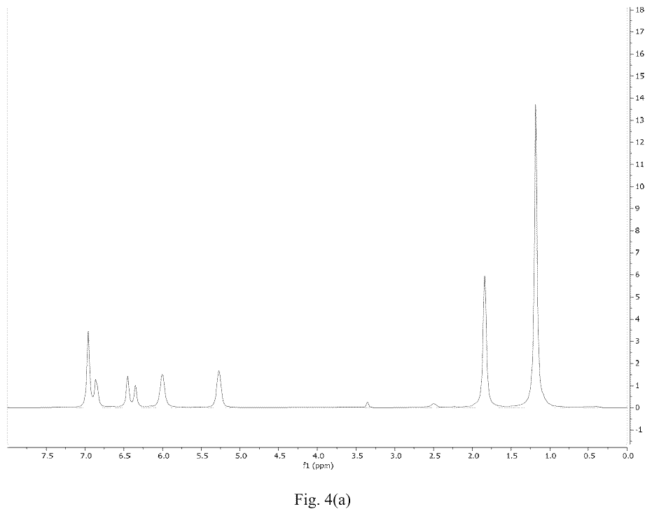

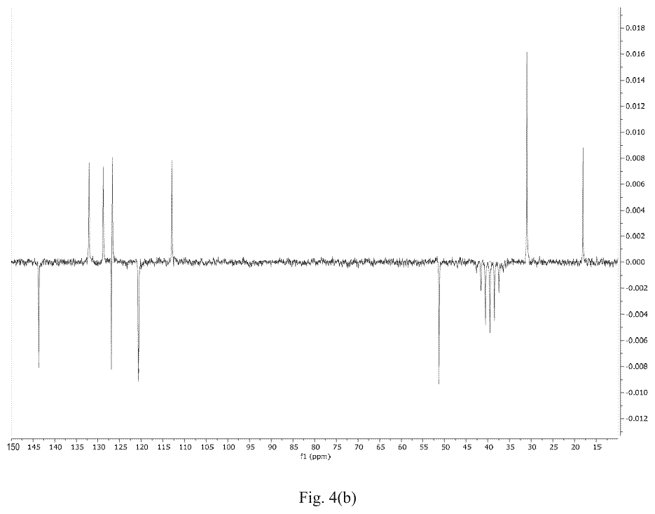

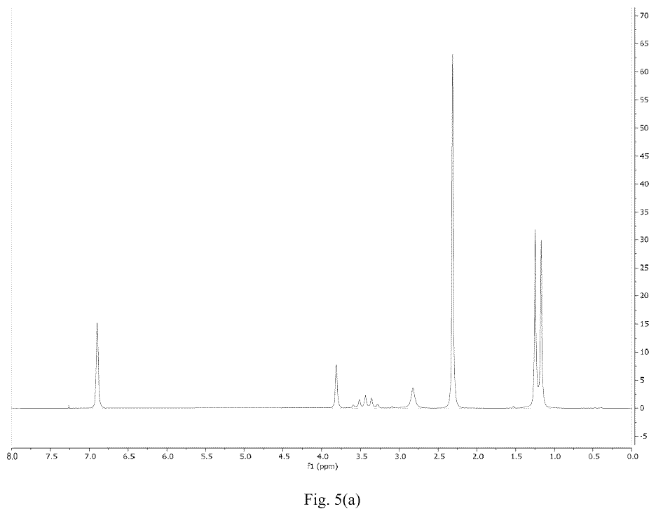

To a 250-mL round bottom flask fitted with a stir-bar and a reflux condenser was loaded the substrate 4,4′-thiodianiline (25.17 g; 116.4 mmol) and acetone (34 mL; 463 mmol). The mixture was stirred under N₂ blanket. The substrate dissolved within minutes. The flask was cooled with a cold tap water bath. To the obtained dark solution was portion-wise added zinc(II) triflate (5.67 g; 15.6 mmol) over 5-6 minutes. The reaction mixture was stirred for 1⅓ hours. The cooling bath was removed. The reaction mixture was stirred without external cooling or heating for about 16 h. The reaction mixture was heated (oil bath temperature=73° C.) for approx. 8 hours, then allowed to cool to room temperature overnight. Mesityl oxide (27 mL; 0.24 mol) was added to the flask. The reaction mixture was refluxed (oil bath temperature=85° C.) for approx. 8 hours then allowed to cool to room temperature overnight. Ethyl acetate (50 mL) was added. The obtained mixture was filtered through pad of Celite. The cake was rinsed with ethyl acetate (10 mL). The combined filtrates were stripped of volatiles under reduced pressure (rotary evaporator; water bath=55° C.). The residue (dark gooey material) was dissolved in toluene (21 mL). The obtained solution was loaded into a 73 mm inner diameter column previously prepared with silica gel 60 and 20% ethyl acetate in hexanes (height of silica=21 cm). The column was eluted with 20% ethyl acetate in hexanes. Each portion coming out was analyzed by silica gel TLC (20% ethyl acetate in hexanes). The portions containing the dot at R_{f}=0.5 were collected, combined, and stripped of volatiles under reduced pressure (rotary evaporator; water bath=50° C.). The obtained light-yellow mass (approx. 14 g) was broken apart into a thin powder. A 10% ethyl acetate in dichloromethane mixture (35 mL) was added and the obtained slurry was stirred (stir-bar) at room temperature for 4 hours. The solid was collected by vacuum filtration, rinsed quickly with several portions of hexanes (total=100 mL), dried under vacuum at 40-50° C. for a few hours then at room temperature overnight to yield 7.97 g (18% with respect to 4,4′-thiodianiline) of Compound 153 as a white powder. Representative ¹H and ¹³C APT NMR spectra are shown in FIGS. 4(a) and 4(b), respectively. LC-UV analysis indicated 98.2% purity at 260 nm detection. The melting point of compound 153 was measured to be about 184° C.

### Example 11

### Synthesis of 4,4′-methylenebis(N-isopropyl-2,6-dimethylaniline) (Compound 154)

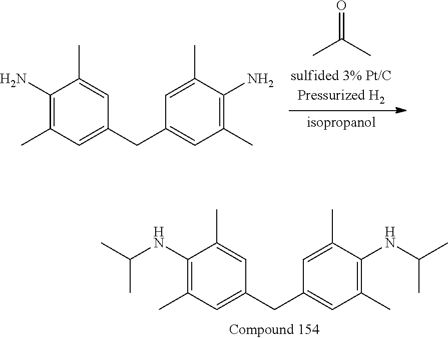

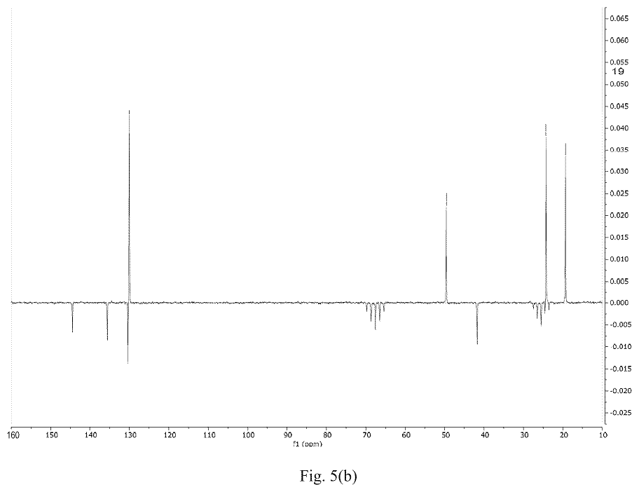

To a 300-mL Parr autoclave was loaded 4,4′-methylenebis(2,6-dimethylaniline) (5.0 g; 19.7 mmol), isopropanol (150 mL), 3% Pt/C sulfided (Johnson Matthey; 40 mg wet), and acetone (4.3 mL; 58.6 mmol). The reaction mixture was heated to 130° C. Once the temperature was reached, the autoclave was continuously fed with H₂ (set pressure=300 PSIG) for several hours. The reaction mixture was allowed to stand at room temperature under N₂ protection overnight. At that stage, approx. 53% monoalkylated intermediates and 27% desired dialkylated product had formed (percentage areas as measured by LC-UV analysis at 260 nm detection). The reaction mixture was heated to 180° C. Once the temperature was reached, the autoclave was continuously fed with H₂ (set pressure=500 PSIG) for approx. 6 hours. Upon cooling to room temperature, the mixture post reaction was filtered through in-line 0.2-micron frit. The clave was rinsed with isopropanol (150 mL) and the mixture was filtered through the inline filter. The combined filtrates were stripped of their volatiles under reduced pressure (rotary evaporator; water bath=55° C. The obtained light-brown oil was diluted with hexanes (5.5 mL). The solution was loaded into a 41 mm inner diameter column previously prepared with silica gel 60 and 15% ethyl acetate in hexanes (height of silica=7.0-7.5″). The column was eluted with 15% ethyl acetate in hexanes. Each portion coming out was analyzed by TLC (15% ethyl acetate in hexanes). The portions containing the dot at R_{f}=0.4 were collected, combined, and stripped volatiles under reduced pressure (rotary evaporator; water bath=50-55° C.). The obtained solid was dried under vacuum at 40-50° C. for a couple of hours, then at room temperature overnight, to afford 1.81 g (27% with respect to 4,4′-methylenebis(2,6-dimethylaniline)) of Compound 154 as a light-pink solid. Representative ¹H and ¹³C APT NMR spectra are shown in FIGS. 5(a) and 5(b), respectively. LC-UV analysis indicated 99.9% purity at 260 nm detection. The melting point of Compound 154 was measured to be about 84-88° C.

### Example 12

### Synthesis of 6,6′-(2-methylpropane-1,1-diyl) bis(2,2,4-trimethyl-1,2-dihydroquinoline) (Compound 159)

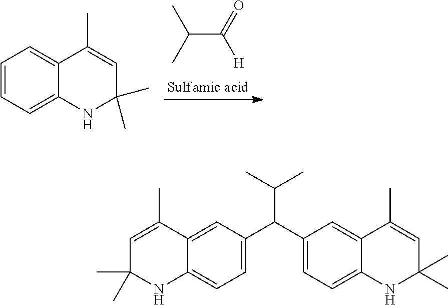

To a stirred solution of 2,2,4-trimethyl-1,2-dihydroquinoline (200 g, 1.1 mol) and isobutyraldehyde (53 mL, 0.57 mol) added sulfamic acid (2.8 g) at RT. The resulting reaction contents were heated at 60° C. and stirred for 24 hr. at 60° C. After completion of the reaction, (monitored by TLC), the reaction was cooled to RT, dissolved in 10% Methanol in DCM (500 mL) and evaporated under reduced pressure to afford 230 g of crude. The obtained crude compound was purified by column chromatography using basic alumina (100-200 mesh) as the stationary phase and eluting with 0-6% ethyl acetate in hexane. The collected fractions were evaporated under vacuum to afford 20.0 g of material as an off-white solid. The solid was further subjected to reverse phase (Biotage) column purification and to afford 2.5 g of 6,6′-(2-methylpropane-1,1-diyl) bis(2,2,4-trimethyl-1,2-dihydroquinoline (1% yield, >96% pure by HPLC 260 nm, Mass (m/z): 401.5 [M+H]⁺). ¹H NMR (400 MHz, DMSO-d₆): δ 6.81 (t, J=6.4 Hz, 4H), 6.33 (d, J=8.8 Hz, 2H), 5.56 (d, J=1.6 Hz, 2H), 5.23 (s, 2H), 2.99 (d J=10.8 Hz, 1H), 2.29-2.26 (m, 1H), 5.69 (s, 2H), 1.87 (s, 6H), 1.13 (s, 12H), 0.77 (d, J=6.4 Hz, 6H). ¹³C NMR (100 MHz, DMSO-d₆): δ 141.92, 132.78, 128.29, 127.50, 127.20, 122.25, 119.89, 112.14, 59.01, 50.96, 31.50, 30.82, 30.76, 21.89, 18.26.

### Example 13

### Synthesis of 6,6′-(ethane-1,1-diyl)bis(2,2,4-trimethyl-1,2-dihydroquinoline) (Compound 160)

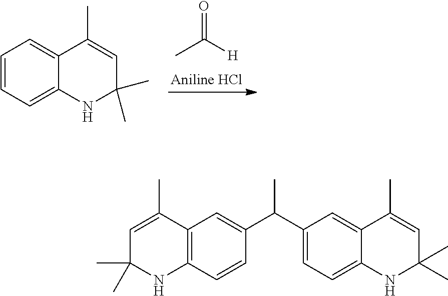

A solution of 2,2,4-trimethyl-1,2-dihydroquinoline (250 g, 1.44 mol) in acetaldehyde (40 mL, 0.7) was placed in seal tube and degassed for 10 min, then anilinium hydrochloride (4.6 g, 0.03 mol) was added. The resulting reaction contents were heated to 50° C. and stirred for 48 hr. After completion of the reaction, (monitored by TLC), reaction mixture was cooled to RT, dissolved in 10% Methanol in DCM (500 mL) and evaporated under reduced pressure to afford 325 g of crude. The above obtained crude compound was purified by column chromatography using basic alumina as the stationary phase, eluting with 0-2% ethyl acetate in hexane. The fractions were collected and evaporated under vacuum to afford 17.0 g of a brown solid. The brown solid was subjected to reverse phase (Biotage) column purification to afford 11.0 g of 6,6′-(ethane-1,1-diyl)bis(2,2,4-trimethyl-1,2-dihydroquinoline) (4% yield, >97% pure by HPLC 260 nm, Mass (m/z): 373.4 [M+H]⁺). ¹H NMR (400 MHz, DMSO-d₆): δ 6.81 (d, J=1.6 Hz, 2H), 6.74 (dd, J=8.0 Hz, 2H), 6.34 (d, J=8.0 Hz, 2H), 5.58 (d, J=1.2 Hz, 2H), 5.24 (s, 2H), 3.74 (J=6.8 Hz, 1H), 1.85 (d, J=1.2 Hz, 6H), 1.42 (d, J=7.2, 3H), 1.15 (d, J=1.2 Hz, 12H). ¹³C NMR (100 MHz, DMSO-d₆): δ 142.04, 133.96, 128.35, 127.51, 126.81, 121.79, 119.85, 112.09, 50.95, 30.73, 22.23, 18.21.

### Example 14

### Synthesis of 6,6′-(phenylmethylene) bis(2,2,4-trimethyl-1,2-dihydroquinoline) (Compound 15)

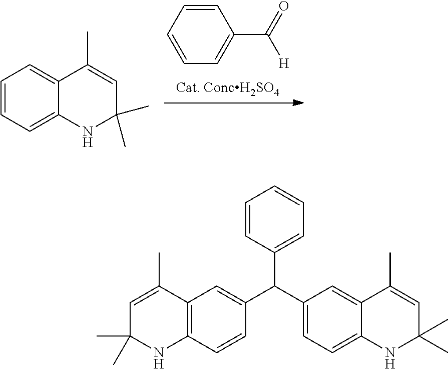

To a stirred solution of 2,2,4-trimethyl-1,2-dihydroquinoline (160 g, 0.92 mol) and benzaldehyde (47.2 mL, 0.46 mol) in ethanol (800 mL) was added drop wise conc. H₂SO₄ (20 mL) over about 15-20 min. at RT and the resulting reaction contents were stirred at RT for 48 hr. After completion of the reaction, (monitored by TLC), reaction mixture was quenched with 1N NaOH solution. The mixture was extracted with DCM (2×250 mL), separated and the combined organic layers were dried over sodium sulphate and evaporated under reduced pressure to obtain 180.0 g of crude. The obtained crude compound was purified by column chromatography using silica gel (100-200 mesh) as the stationary phase, eluting with 0-6% ethyl acetate in hexane. The fractions were evaporated under vacuum to afford 46.0 g of Compound 15 (11% yield, >96% pure by HPLC 260 nm, Mass (m/z): 435.31[M+H]⁺) as an off-white solid. ¹H NMR (400 MHz, DMSO-d₆): δ 7.25 (t, J=7.2 Hz, 2H), 7.14 (t, J=7.2 Hz, 2H), 7.09 (d, J=7.2 Hz, 2H), 6.72 (d, J=1.6 Hz, 2H), 6.61 (dd J=8.0 Hz, 2H), 6.36 (d, J=8.4 Hz, 2H), 5.69 (s, 2H), 5.24 (s, 2H), 5.16 (s, 1H), 1.76 (s, 6H), 1.16 (s, 12H). ¹³C NMR (100 MHz, DMSO-d₆): δ 146.19, 142.75, 131.70, 129.28, 129.23, 128.96, 128.43, 127.86, 126.07, 124.17, 120.29, 112.50, 55.45, 51.55, 31.34, 18.66.

### Example 15

### Synthesis of N-phenyl-4-((2,2,4-trimethyl-1,2-dihydroquinolin-6-yl) methyl) aniline (Compound

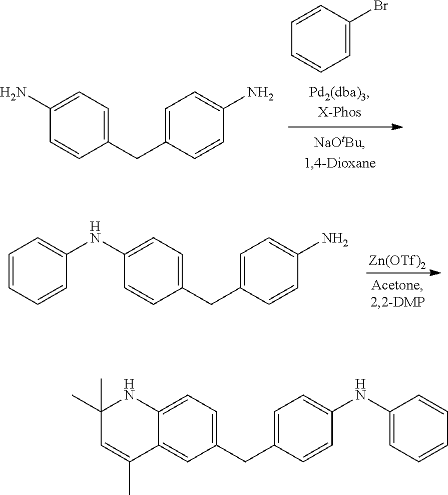 [SMILES: Brc1ccccc1 ; Nc1ccc(Cc2ccc(N)cc2)cc1 ; Nc1ccc(Cc2ccc(Nc3ccccc3)cc2)cc1 ; CC1=CC(C)(C)Nc2ccc(Cc3ccc(Nc4ccccc4)cc3)cc21 ; "Zn(OTf)2"]

### Step 1: Synthesis of 4-(4-aminobenzyl)-N-phenylaniline

To a stirred solution of 4,4′-methylenedianiline (50.0 g, 0.25 mol) in 1,4-Dioxane (500 mL, 10 vol) was added sodium tertiary butoxide (36.40 g, 0.37 mol) and X-Phos (4.81 g, 0.01 mol) at room temperature under nitrogen atmosphere. The resulting mixture was degassed for 20 minutes with nitrogen gas, then Pd₂(dba)₃ was added. The resulting reaction mixture was stirred at 100° C. for 16 h. The progress of reaction was monitored by TLC. After completion of the reaction by TLC, the mixture was cooled to room temperature and filtered through celite pad under vacuum. Filtrate was evaporated under reduced pressure to get the crude compound. The obtained crude compound was purified by basic silica gel (100-200 mesh) column chromatography, eluted with 20% EtOAc/hexane. The fractions were evaporated to afford 25 g of 4-(4-aminobenzyl)-N-phenylaniline as a brown liquid.

### Step 2: Synthesis of N-phenyl-4-((2,2,4-trimethyl-1,2-dihydroquinolin-6-yl) methyl) aniline

To a stirred solution of 4-(4-aminobenzyl)-N-phenylaniline (40.0 g, 0.14 mol) in acetone (40 mL, 1 vol) and 2,2-dimethoxypropane (40 mL, 1 vol) was added zinc triflate (10.60 g, 0.02 mol) under nitrogen atmosphere at room temperature. The resulting reaction mixture was stirred at room temperature for 48 h. The progress of the reaction was monitored by TLC. After completion of the reaction, solvent was concentrated under vacuum. The obtained residue was purified by silica gel (100-200 mesh), eluted with 2-3% EtOAc in hexane and the fractions were evaporated under vacuum to afford 25.0 g of N-phenyl-4-((2,2,4-trimethyl-1,2-dihydroquinolin-6-yl) methyl) aniline (48% yield, >99% pure by HPLC 260 nm, Mass (m/z): 355.39 [M+H]⁺) as a pale-yellow solid. ¹H NMR (400 MHz, DMSO-d₆): δ 8.01 (s, 1H), 7.17 (t, J=8.4 Hz, 2H), 7.05 (d, J=8.4 Hz, 2H), 6.99 (t, J=0.84 Hz, 4H), 6.81 (s, 1H), 6.74 (m, 2H), 6.37 (d, J=8.0 Hz, 1H), 5.63 (s, 1H), 5.25 (s, 1H), 3.65 (s, 2H), 1.86 (s, 3H), 1.16 (s, 6H). ¹³C NMR (100 MHz, DMSO-d₆): δ 144.39, 142.76, 141.42, 134.37, 129.59, 128.99, 128.94, 128.77, 127.95, 123.84, 120.64, 119.51, 117.80, 116.47, 112.82, 51.50, 40.61, 40.40, 31.24, 18.75.

### Example 16

### Synthesis of N-benzyl-4-((2,2,4-trimethyl-1,2-dihydroquinolin-6-yl) methyl) aniline (Compound

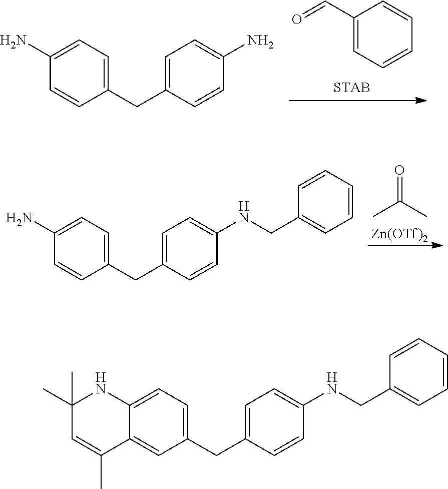

### Step 1: Synthesis of 4-(4-aminobenzyl)-N-benzyl aniline

To the stirred solution of 4,4′-methylenedianiline (100 g, 0.50 mol) in EDC (2000 mL) was added benzaldehyde (51 mL, 0.50 mol) at RT, then was added NaOAc (33.5 g. 0.50 mol). STAB (268 g, 1.26 mol) was added over a 30-40 minutes interval, and the reaction was maintained for 16 h at RT. After completion of the reaction (monitored by TLC) it quenched with NaHCO₃ solution and extracted with DCM (2×200 ml). The combined the organic layers were washed with sat NaCl solution, dried over Na₂SO₄ and solvent was evaporated under reduced pressure to give the 100 g of crude. The obtained crude compound was purified by column chromatography (silica gel 100-200 mesh) eluting with 5-10% EtOAc in hexane to afford 65 g of 4-(4-aminobenzyl)-N-benzyl aniline as brown liquid.

### Step 2: Synthesis of N-benzyl-4-((2,2,4-trimethyl-1,2-dihydroquinolin-6-yl) methyl) aniline

To a solution of 4-(4-aminobenzyl)-N-benzyl aniline (90.0 g, 0.20 mol) in DMP (450 mL) was added zinc triflate (33.3 g, 0.093 mol) under nitrogen atmosphere at RT. The resulting mixture was stirred for 3 days. After completion of the reaction by TLC, solvent was evaporated under reduced pressure to give crude product. This crude compound was purified by column chromatography (silica gel 100-200 mesh) eluting with 3-5% EtOAc in hexane to afford 42.6 g of N-benzyl-4-((2,2,4-trimethyl-1,2-dihydroquinolin-6-yl) methyl) aniline (37% yield, >98% pure by HPLC 260 nm, Mass (m/z): 369.40 [M+H]⁺) as brown liquid. ¹H-NMR (400 MHz, DMSO-d₆): δ 7.34-7.27 (m, 4H), 7.18 (t, J=5.6 Hz, 1H), 6.85 (d, J=8.4 Hz, 2H), 6.75 (d, J=1.6 Hz, 1H), 6.66 (dd, J=2.0, 8.0 Hz, 1H), 6.46 (d, J=8.4 Hz, 2H), 6.33 (d, J=8.0 Hz, 1H), 6.03 (t, J=6.0 Hz, 1H), 5.59 (s, 1H), 5.23 (s, 1H), 4.20 (d, J=6.0 Hz, 2H), 3.54 (s, 2H), 1.83 (s, 3H), 1.15 (s, 6H). ¹³C NMR (100 MHz, DMSO-d₆): δ 147.13, 142.64, 140.93, 129.98, 129.34, 128.94, 128.84, 128.70, 127.99, 127.61, 127.0, 123.72, 120.60, 112.78, 51.49, 47.17, 40.61, 40.40, 39.36, 31.20, 18.76.

### Example 17

### Synthesis of N-(5-methylhexan-2-yl)-4-((2,2,4-trimethyl-1,2-dihydroquinolin-6yl)methyl)aniline (Compound 164)

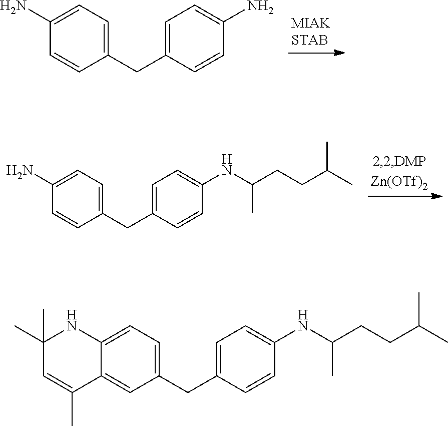

### Step 1: 4-(4-aminobenzyl)-N-(5-methylhexan-2-yl) aniline

To a stirred solution of 4,4′-methylenedianiline (100 g, 0.50 mol) in MeOH (500 mL) and AcOH (500 mL) was added MIAK (106 mL, 0.75 mol) at RT. STAB (321.2 g, 1.5 mol) was added portion-wise over 30-40 min and the resulting reaction contents were maintained for 6 h at RT. After completion of the reaction (monitored by TLC), the reaction mixture was evaporated, quenched with NaHCO₃ solution and extracted with DCM (2×500 ml). The combined the organic layers were washed with sat. NaCl solution and dried over Na₂SO₄. After filtration, the solvent was evaporated under reduced pressure obtaining a crude compound which was purified by column chromatography (silica gel 100-200 mesh) eluting with 2-3% EtOAc in hexane to afford 64 g of 4-(4-aminobenzyl)-N-(5-methylhexan-2-yl) aniline (Light Brown liquid).

### Step 2: N-(5-methylhexan-2-yl)-4-((2,2,4-trimethyl-1,2-dihydroquinolin-6yl)methyl)aniline

To the solution of 4-(4-aminobenzyl)-N-(5-methylhexan-2-yl) aniline (70 g, 0.23 mol) in 2,2-DMP (350 mL) was added zinc triflate (25.7 g, 0.07 mol) under nitrogen atmosphere at RT. The resulting reaction contents were maintained for 96 h at RT. After completion of the reaction by TLC, solvent was evaporated under reduced pressure to give crude material. The obtained crude compound was purified by column chromatography (silica gel 100-200 mesh) eluting with 2-3% EtOAc in hexane to afford 31.8 g of N-(5-methylhexan-2-yl)-4-((2,2,4-trimethyl-1,2-dihydroquinolin-6yl)methyl)aniline (36% yield, >98% pure by HPLC 260 nm, Mass (m/z): 377.52 [M+H]⁺) as a yellow oil. ¹H-NMR (400 MHz, DMSO-D₆): δ 6.85 (d, J=8.4 Hz, 2H), 6.76 (d, J=1.6 Hz, 1H), 6.68 (dd, J=1.6, 8.0 Hz, 1H), 6.43 (d, J=8.4 Hz, 2H), 6.34 (d, J=8.0 Hz, 1H), 5.59 (d, J=0.8 Hz, 1H), 5.24 (s, 1H), 5.06 (d, J=8.4 Hz, 1H), 3.55 (s, 2H), 3.33-3.25 (m, 1H), 1.84 (d, J=0.8 Hz, 3H), 1.51-1.17 (m, 3H), 1.15 (s, 6H), 1.04 (d, J=6.4 Hz, 3H), 0.85-0.83 (m, 6H). ¹³C NMR (100 MHz, DMSO-d₆): δ 146.80, 142.62. 129.41, 129.19, 128.92, 128.82, 127.98, 123.70, 120.59, 112.75, 51.47, 48.17, 40.61, 40.40, 40.20, 35.48, 34.59, 31.19, 28.13, 23.09, 23.02, 20.91, 18.75.

### Example 18

### Synthesis of N-(1-phenylethyl)-4-((2,2,4-trimethyl-1,2-dihydroquinolin-6-yl) methyl) aniline (Compound 165)

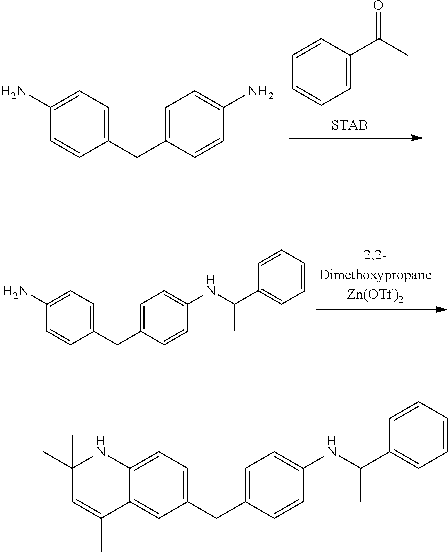

### Step 1: Synthesis of 4-(4-aminobenzyl)-N-(1-phenylethyl) aniline

To a stirred solution of 4,4′-methylenedianiline (100 g, 0.50 mol) in MeOH (1000 mL) and AcOH (200 mL) was added acetophenone (58.7 mL, 0.50 mol) at RT. NaCNBH₃ (62.5 g, 1.0 mol) was added portion-wise in 3 lots each over 30-40 min intervals and the resulting reaction contents were maintained for 6-8 h at RT. After completion of the reaction (monitored by TLC) the reaction mixture was evaporated under reduced pressure to obtain a residue and which was quenched with NaHCO₃, extracted with ethyl acetate (2×500 ml), and the combined the organic layers washed with sat. NaCl solution. The organic layer was separated, dried over Na₂SO₄ and evaporated under reduced pressure afforded the crude compound. The obtained crude material was purified by column chromatography to afford pure 59 g of 4-(4-aminobenzyl)-N-(1-phenylethyl) aniline.

### Step 2: Synthesis of N-(1-phenylethyl)-4-((2,2,4-trimethyl-1,2-dihydroquinolin-6-yl) methyl) aniline

To a solution of 4-(4-aminobenzyl)-N-(1-phenylethyl) aniline (70.0 g, 0.277 mol) in 2,2-dimethoxy propane (400 mL) was added zinc triflate (20 g, 0.055 mol) under nitrogen atmosphere at RT. The resulting reaction contents were stirred for 72 h at RT. After completion of the reaction by TLC, solvent was evaporated under reduced pressure to give crude compound. The obtained crude compound was purified by column chromatography (silica gel 60-120 mesh) eluting with 2-3% EtOAc in hexane to afford 42 g N-(1-phenylethyl)-4-((2,2,4-trimethyl-1,2-dihydroquinolin-6-yl) methyl) aniline (42% yield, >99% pure by HPLC 260 nm, Mass (m/z): 383.2 [M+H]⁺) as an off white solid. ¹H-NMR (400 MHz, DMSO-d₆): δ 7.35 (d, J=1.2 Hz, 2H), 7.26 (t, J=7.6 Hz, 2H), 7.16-7.13 (m, 1H), 6.78 (d, J=8.4 Hz, 2H), 6.72 (d, J=1.2 Hz, 1H), 6.63 (dd, J=2.0, 6.0 Hz, 1H), 6.38 (d, J=8.4 Hz, 2H), 6.32 (d, J=8.0 Hz, 1H), 5.95 (d, J=6.8 Hz, 1H), 5.58 (s, 1H), 5.22 (s, 1H), 4.42-4.35 (m, 1H), 3.50 (s, 2H), 1.82 (s, 3H), 1.37 (d, J=6.8 Hz 3H), 1.14 (s, 6H). ¹³C-NMR (100 MHz, DMSO-d₆): δ 146.75, 146.39, 142.61, 129.75, 129.24, 129.15, 128.91, 128.82, 128.74, 127.97, 126.80, 126.35, 123.70, 120.55, 113.25, 112.74, 52.68, 51.47, 40.61, 31.20, 25.24, 18.74.

### Example 19

### Synthesis of N-isopropyl-4-((2,2,4-trimethyl-1,2-dihydroquinolin-6-yl) methyl) aniline (Compound 166)

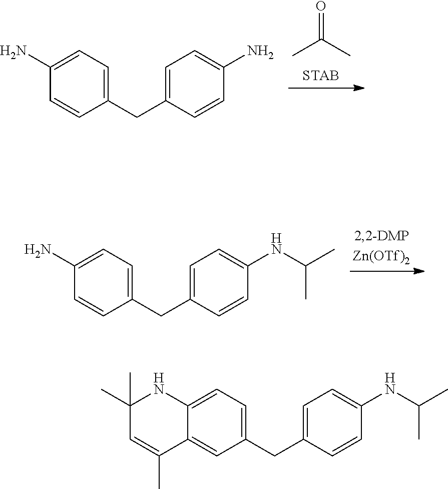

### Step 1: Synthesis of 4-(4-aminobenzyl)-N-isopropyl aniline

To a stirred solution of 4,4′-methylenedianiline (100 g, 0.50 mol) in MeOH (1000 mL) and AcOH (200 mL) was added acetone (37.5 mL, 0.50 mol) at RT. STAB (321 g, 1.51 mol) was added portion-wise over 30-40 min and the reaction was maintained at RT for 6-8 h. After completion of the reaction (monitored by TLC) the reaction mixture was concentrated, quenched with NaHCO₃ solution and extracted with ethyl acetate (2×500 ml). Combined organic layers were washed with sat. NaCl solution. The organic layer was separated, dried over Na₂SO₄ and evaporated under reduced pressure to afford crude. The obtained crude compound was purified by column chromatography to afford 52 g of 4-(4-aminobenzyl)-N-isopropyl aniline.

### Step 2: Synthesis of N-isopropyl-4-((2,2,4-trimethyl-1,2-dihydroquinolin-6-yl) methyl) aniline

To a solution of 4-(4-aminobenzyl)-N-isopropyl aniline (40.0 g, 0.16 mol) in 2,2-Dimethoxypropane (200 mL) was added zinc triflate (12 g, 0.033 mol) under nitrogen atmosphere at RT. The resulting mixture was maintained for 72 h at RT. After completion of the reaction by TLC, solvent was evaporated under reduced pressure to give crude compound. The obtained crude compound was purified by column chromatography (silica gel 60-120 mesh) eluting with 6-8% EtOAc in hexane to afford 23 g of N-isopropyl-4-((2,2,4-trimethyl-1,2-dihydroquinolin-6-yl) methyl) aniline (43% yield, >98% pure by HPLC 260 nm, Mass (m/z): 321.2 [M+H]⁺) as off white solid. ¹H NMR (400 MHz, DMSO-d₆): δ 6.87 (d, J=8.4 Hz, 2H), 6.77 (d, J=1.2 Hz, 1H), 6.69-6.67 (dd, J=2.0, 2.0 Hz, 1H), 6.44 (d, J=8.4 Hz, 2H), 6.34 (d, J=8.0 Hz, 1H), 5.59 (s, 1H), 5.24 (s, 1H), 5.08 (d, J=8.0 Hz, 1H), 3.55 (s, 2H), 3.50-3.42 (m, 1H), 1.84 (s, 3H), 1.15 (s, 6H), 1.08 (d, J=6.4 Hz, 6H). ¹³C NMR (100 MHz, DMSO-d₆): δ 146.60, 142.63, 129.41, 128.94, 128.83, 127.98, 123.71, 120.59, 112.94, 112.76, 51.48, 43.57, 40.61, 40.40, 40.19, 39.98, 31.19, 23.00, 18.75.

### Example 20

### Use of Compounds of the Disclosure in Sidewall Formulations

In this study, rubber compounds were prepared using Compounds of the Disclosure as antidegradants in sidewall formulations. As shown in Table 2, these rubber compounds are a 50/50 blend of natural rubber and butadiene rubber with a carbon black of the N500 series. Sidewall compounds with Compounds of the Disclosure or no antidegradant can be seen in the Examples listed in Table 2. According to American Society for Testing Materials (ASTM) test method D5289, vulcanization properties of all the compounds were characterized by a moving die rheometer (Alpha Technologies MDR 2000), also known as MDR, at a temperature of 160° C. for 60 minutes. The oscillation strain and frequency were set to 0.5° C. and 1.667 Hz, respectively. The MDR test results are summarized in Table 3.

TABLE 2

| | Example # | | | | | | | | | |
|---|---|---|---|---|---|---|---|---|---|---|
| Material | 1 | 2 | 3 | 3a | 7 | 8 | 16 | 19 | 20 | 21 |
| Natural | 50 | 50 | 50 | 70 | 70 | 70 | 70 | 70 | 70 | 70 |
| rubber (SIR- | | | | | | | | | | |
| 10) | | | | | | | | | | |
| Butadiene | 50 | 50 | 50 | 30 | 30 | 30 | 30 | 30 | 30 | 30 |
| rubber | | | | | | | | | | |
| (CB24) | | | | | | | | | | |
| N-550 | 50 | 50 | 50 | 80 | 80 | 80 | 80 | 80 | 80 | 80 |
| Carbon Black | | | | | | | | | | |
| Stearic acid | 2 | 2 | 2 | 8 | 8 | 8 | 8 | 8 | 8 | 8 |
| Calsol 8240 | 6.25 | 6.25 | 6.25 | 10 | 10 | 10 | 10 | 10 | 10 | 10 |
| (Naphthenic | | | | | | | | | | |
| Oil) | | | | | | | | | | |
| Zinc oxide | 4 | 4 | 4 | 17 | 17 | 17 | 17 | 17 | 17 | 17 |
| Sulfur | 1.5 | 1.5 | 1.5 | 1 | 1 | 1 | 1 | 1 | 1 | 1 |
| TBBS | 0.6 | 0.6 | 0.6 | 3 | 3 | 3 | 3 | 3 | 3 | 3 |
| Compound 1 | 2.68 | — | — | — | — | — | — | — | — | — |
| Compound 2 | — | 2.71 | — | — | — | — | — | — | — | — |
| Compound 3 | — | — | 2.6 | 2.6 | — | — | — | — | — | — |
| Compound 7 | — | — | — | — | 1.1 | — | — | — | — | — |
| Compound 8 | — | — | — | — | — | 1.37 | — | — | — | — |
| Compound 16 | — | — | — | — | — | — | 1.37 | — | — | — |
| Compound 19 | — | — | — | — | — | — | — | 2.31 | — | — |
| Compound 20 | — | — | — | — | — | — | — | — | 1.46 | — |
| Compound 21 | — | — | — | — | — | — | — | — | — | 2.05 |
| Total parts | 167.03 | 167.06 | 166.95 | 221.6 | 220.1 | 220.37 | 220.37 | 221.31 | 220.46 | 221.05 |
| per hundred | | | | | | | | | | |
| rubber (phr) | | | | | | | | | | |

TABLE 3

| | Material (Example #) | | | | | | | | | | |
|---|---|---|---|---|---|---|---|---|---|---|---|
| | 1 | 2 | 3 | 1 | 3 | 7 | 8 | 16 | 19 | 20 | 21 |
| Temperature | 150 | 150 | 150 | 160 | 160 | 160 | 160 | 160 | 160 | 160 | 160 |
| (° C.) | | | | | | | | | | | |
| ML (dNm) ¹ | 2.38 | 2.53 | 2.48 | 2.2 | 2.2 | 2.2 | 2.2 | 2.2 | 2.1 | 2.1 | 2.1 |
| MH (dNm) ² | 15.43 | 15.18 | 14.8 | 15.4 | 14.1 | 14.1 | 13.8 | 15.2 | 14.1 | 13.6 | 14.7 |
| tMH (min) ³ | — | — | — | 12.0 | 12.8 | 15.0 | 15.9 | 13.8 | 15.7 | 17.8 | 15.9 |
| Ts₁ (min) ⁴ | — | — | — | 3.8 | 3.7 | 4.2 | 4.5 | 3.8 | 4.4 | 4.7 | 4.5 |
| Ts₂ (min) ⁵ | 7.51 | 5.87 | 7.06 | 4.3 | 4.2 | 4.9 | 5.1 | 4.6 | 5.2 | 5.6 | 5.3 |
| T₁₀ (min) ⁶ | 7.05 | 5.47 | 6.59 | 4.0 | 3.8 | 4.4 | 4.6 | 4.2 | 4.7 | 4.9 | 4.8 |
| T₂₅ (min) ⁷ | — | — | — | 4.6 | 4.6 | 5.3 | 5.5 | 5.0 | 5.6 | 6.0 | 5.8 |
| T₅₀ (min) ⁸ | 9.6 | 7.75 | 9.04 | 5.4 | 5.5 | 6.3 | 6.7 | 5.8 | 6.7 | 7.3 | 6.9 |
| T₉₀ (min) ⁹ | 14.84 | 12.67 | 14.09 | 8.1 | 8.4 | 9.7 | 10.3 | 8.8 | 10.2 | 11.2 | 10.4 |

¹ ML is the minimum torque, which is a measure of rigidity and viscosity of the non-vulcanized compound.
² MH is the maximum torque, which is a measure of the cross-linking density of the fully vulcanized compound.
³ tMH is the time to reach maximum torque MH
⁴ Ts₁ is the time for viscosity to increase 1 unit (dNm) above ML.
⁵ Ts₂ is the time for viscosity to increase 2 units (dNm) above ML.
⁶ T₁₀ is the moment when 10% cross-linking has been reached.
⁷ T₂₅ is the moment when 25% cross-linking has been reached.
⁸ T₅₀ is the moment when 50% cross-linking has been reached.
⁹ T₉₀ is the moment when 90% cross-linking has been reached.

### Example 21

### Antiozonant Performance of Compounds of the Disclosure in Sidewall Compounds

Each of the rubber compounds prepared in Example 20 were cured at 150° C. or 160° C. to a state equivalent to its T₉₀ time. The ozone test specimens were died out from vulcanized tensile sheets by using a die followed the American Society for Testing and Materials (ASTM) D1329 standard. The ozone test specimens were stored at ambient environment for more than 24 hours prior to antiozonant performance testing in an ozone chamber, which was maintained at an ozone concentration of 10±2 parts per hundred million (pphm) and 40±2° C. The ozone test specimens were simultaneously relaxed and extended within ozone chamber under three different conditions: static, intermittent, and dynamic (150° C. test) or under dynamic condition (160° C. test). The ozone test specimens were maintained at 25% strain during static exposure and alternately relaxed and extended to 25% strain at a rate of 90 cycles per minute during dynamic exposure. The intermittent test alternated 18 minutes of dynamic exposure and 102 minutes of static exposure. Exposure was in increments of 16 hours followed by at least one hour of relaxation after removal from the ozone chamber and prior to measurement of force at a strain of 100%.

Antiozonant performance of antidegradant formulated compounds were evaluated by percentage of force retention. Percentage of force retention is defined by equation 1.

$$Percentage of force retention = \frac{F}{{F}_{0}} * 100 \% \qquad Equation l$$

wherein F₀ is the force at a strain of 100% prior to ozone testing and F is the force at a strain of 100% after samples are aged in ozone chamber for 16-hour cycle at static, intermittent, or dynamic exposure. Higher percentage of force retention is an indication of better antiozonant performance of an antidegradant compound incorporated in the vulcanized rubber compound.

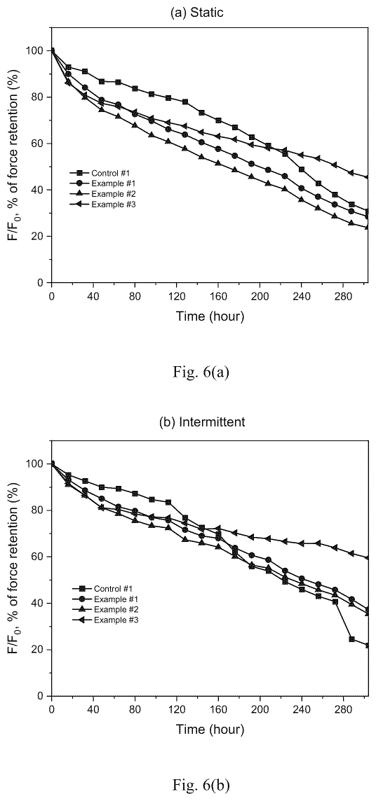

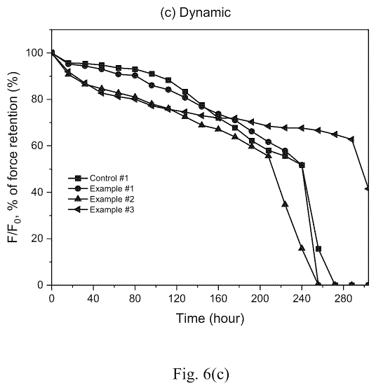

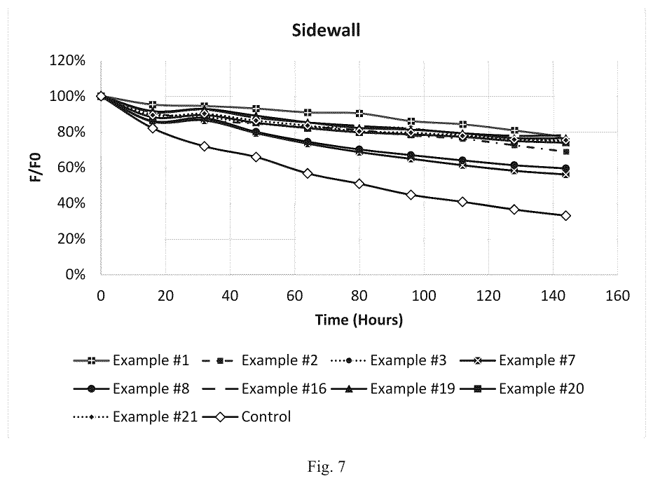

The force of aged and unaged ozone test specimens was measured by using a tensometer (Alpha Technologies, 2000) at a strain of 100%. The percentage of force retention were calculated and plotted as a function of ozone aging time in FIGS. 6(a)-6(c) (150° C. test) and FIG. 7 (160° C. test).

### Example 22

### Oxidative Induction Time (OIT) Performance of Compounds of the Disclosure

In order to demonstrate the antioxidant efficacy of the Compounds of the Disclosure, the oxidative induction time (OIT) of selected examples were evaluated. OIT is measured according to a procedure carried out in a differential scanning calorimeter (TA Instruments, Q200), also knows as DSC, and is used to predict thermo-oxidative performance of a material. In this procedure, each sample is held in a sample cell and heated under a nitrogen atmosphere to a preselected temperature. Oxygen is then introduced to the sample cell and the length of time before the onset of degradation, as seen by the initiation of increase in heat flow, is measured.

0.5 wt % of a Compound of the Disclosure, N¹-(4-methylpentan-2-yl)-N⁴-phenylbenzene-1,4-diamine (6PPD), or no antidegradant (blank) was mixed with polyisoprene and heated isothermally at 150° C. and/or 160° C. in 02. The peak time of OIT testing in minutes is shown in Table 4. As indicated by the data in Table 4, the molecules of the present invention demonstrate some antioxidant performance compared against blank control and indicates utility in rubber and other applications that can benefit from an active antioxidant.

TABLE 4

| | Peak time of OIT testing | |
|---|---|---|
| | (minutes) | |
| Material | 150° C. | 160° C. |
| Blank | 1.6-1.9 | 1.2-1.4 |
| 6PPD | 42.1 | 21.7 |
| Compound 1 | — | 4.8 |
| Compound 2 | — | 10.8 |
| Compound 3 | — | 5.9 |
| Compound 7 | 7.7 | 4.1 |
| Compound 8 | 4.1 | 2.9 |
| Compound 11 | 5.0 | 2.5 |
| Compound 16 | 9.7 | 6.6 |
| Compound 19 | 5.4 | 2.7 |
| Compound 20 | 4.3 | 2.3 |
| Compound 21 | 4.3 | 2.6 |
| Compound 27 | 8.9 | 4.2 |
| Compound 153 | 12.8 | 5.8 |
| Compound 154 | 2.2 | 1.5 |
| Compound 159 | 13.0 | 5.5 |
| Compound 160 | 12.7 | 5.3 |
| Compound 15 | 16.2 | 7.1 |
| Compound 162 | 27.6 | 12.2 |
| Compound 163 | 9.6 | 5.5 |
| Compound 164 | 9.7 | 4.9 |
| Compound 165 | 7.2 | 4.4 |
| Compound 166 | 11.2 | 4.3 |

### Example 23

### Use of of Compounds of the Disclosure in Tread Compounds

Rubber compounds were prepared using Compounds of the Disclosure as antidegradants in tread formulations. As shown in Table 5, these rubber compounds are a 70/30 blend of styrene-butadiene rubber and butadiene rubber with silica and carbon black. According to American Society for Testing Materials (ASTM) test method D5289, vulcanization properties of all the compounds were characterized by a moving die rheometer (Alpha Technologies MDR 2000), also known as MDR, at a temperature of 160° C. for 60 minutes. The oscillation strain and frequency were set to 0.5° C. and 1.667 Hz, respectively. The MDR test results are summarized in Table 6.

TABLE 5

| | Example # | | | | | | | |
|---|---|---|---|---|---|---|---|---|
| Material | 1 | 3 | 7 | 8 | 16 | 19 | 20 | 21 |
| Duradene 738 | 70 | 70 | 70 | 70 | 70 | 70 | 70 | 70 |
| (SBR) | | | | | | | | |
| Firestone | 30 | 30 | 30 | 30 | 30 | 30 | 30 | 30 |
| 140ND (BR) | | | | | | | | |
| Ultrasil | 80 | 80 | 80 | 80 | 80 | 80 | 80 | 80 |
| 7000GR | | | | | | | | |
| Si69 Silane | 8 | 8 | 8 | 8 | 8 | 8 | 8 | 8 |
| N330 Carbon | 10 | 10 | 10 | 10 | 10 | 10 | 10 | 10 |
| black | | | | | | | | |
| TDAE Oil | 17 | 17 | 17 | 17 | 17 | 17 | 17 | 17 |
| Stearic Acid | 1 | 1 | 1 | 1 | 1 | 1 | 1 | 1 |
| Zinc Oxide | 3 | 3 | 3 | 3 | 3 | 3 | 3 | 3 |
| Sulfur (RMS) | 1.5 | 1.5 | 1.5 | 1.5 | 1.5 | 1.5 | 1.5 | 1.5 |
| CBS | 1.5 | 1.5 | 1.5 | 1.5 | 1.5 | 1.5 | 1.5 | 1.5 |
| DBG | 2 | 2 | 2 | 2 | 2 | 2 | 2 | 2 |
| TDAE oil | 3 | 3 | 3 | 3 | 3 | 3 | 3 | 3 |
| Compound 1 | 2.67 | — | — | — | — | — | — | — |
| Compound 3 | — | 2.6 | — | — | — | — | — | — |
| Compound 7 | — | — | 1.1 | — | — | — | — | — |
| Compound 8 | — | — | — | 1.37 | — | — | — | — |
| Compound 16 | — | — | — | — | 1.37 | — | — | — |
| Compound 19 | — | — | — | — | — | 2.31 | — | — |
| Compound 20 | — | — | — | — | — | — | 1.46 | — |
| Compound 21 | — | — | — | — | — | — | — | 2.05 |

TABLE 6

| | Material (Example #1) | | | | | | | |
|---|---|---|---|---|---|---|---|---|
| | 1 | 3 | 7 | 8 | 16 | 19 | 20 | 21 |
| Temperature | 160 | 160 | 160 | 160 | 160 | 160 | 160 | 160 |
| (° C.) | | | | | | | | |
| ML (dNm) ¹ | 3.1 | 3.1 | 2.6 | 2.5 | 2.5 | 2.5 | 2.5 | 2.5 |
| MH (dNm) ² | 38.1 | 29.7 | 29.5 | 29.0 | 29.5 | 29.3 | 29.5 | 29.2 |
| tMH (min) ³ | 59.9 | 59.7 | 59.7 | 59.4 | 59.7 | 59.8 | 60.0 | 59.5 |
| Ts1 (min) ⁴ | 0.8 | 0.4 | 0.5 | 0.5 | 0.5 | 0.5 | 0.5 | 0.5 |
| Ts2 (min) ⁵ | 1.1 | 0.8 | 1.0 | 1.1 | 1.1 | 1.1 | 1.1 | 1.2 |
| T10 (min) ⁶ | 1.6 | 1.2 | 1.5 | 1.6 | 1.7 | 1.7 | 1.6 | 1.7 |
| T25 (min) ⁷ | 2.4 | 2.6 | 3.5 | 3.9 | 4.0 | 4.1 | 4.0 | 4.3 |
| T50 (min) ⁸ | 3.8 | 3.9 | 5.0 | 5.5 | 5.6 | 5.8 | 5.6 | 6.0 |
| T90 (min) ⁹ | 30.1 | 23.8 | 17.4 | 18.5 | 18.4 | 18.5 | 18.6 | 19.4 |

¹ ML is the minimum torque, which is a measure of rigidity and viscosity of the non-vulcanized compound.
² MH is the maximum torque, which is a measure of the cross-linking density of the fully vulcanized compound.
³ tMH is [the time to reach maximum torque MH
⁴ Ts₁ is the time for viscosity to increase 1 unit (dNm) above ML.
⁵ Ts₂ is the time for viscosity to increase 2 units (dNm) above ML.
⁶ T₁₀ is the moment when 10% cross-linking has been reached.
⁷ T₂₅ is the moment when 25% cross-linking has been reached.
⁸ T₅₀ is the moment when 50% cross-linking has been reached.
⁹ T₉₀ is the moment when 90% cross-linking has been reached.

### Example 24

### Use of of Compounds of the Disclosure in Tread Compounds

Each of the rubber compounds prepared in Example 23 were cured at 160° C. to a state equivalent to its T90+2 min time. The ozone test specimens were died out from vulcanized tensile sheets by using a die followed the American Society for Testing and Materials. (ASTM) D1329 standard. The ozone test specimens were stored in an ambient environment for more than 24 hours prior to antiozonant performance testing in an ozone chamber, which was maintained at an ozone concentration of 10+2 parts per hundred million (pphm) and 40+2° C. The ozone test specimens were simultaneously relaxed and extended within ozone chamber under dynamic conditions. The ozone test specimens extended to 25% strain at a rate of 90 cycles per minute during dynamic exposure. Exposure was in increments of 16 hours followed by at least one hour of relaxation after removal from the ozone chamber and prior to measurement of force at a strain of 100%.

Antiozonant performance of antidegradant formulated compounds were evaluated by percentage of force retention. The percentage of force retention is defined by Equation 1 above.

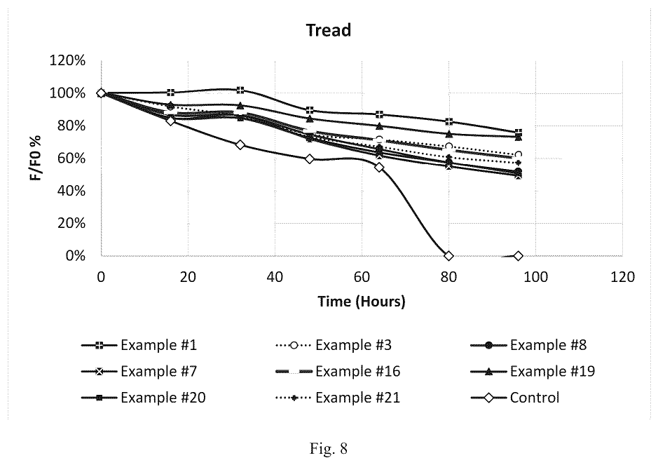

The force of aged and unaged ozone test specimens was measured by using a tensometer (Alpha Technologies, 2000) at a strain of 100%. The percentage of force retention were calculated and plotted as a function of ozone aging time in FIG. 8.

Having now fully described the methods, compounds, and compositions herein, it will be understood by those of skill in the art that the same can be performed within a wide and equivalent range of conditions, formulations, and other parameters without affecting the scope of the methods, compounds, and compositions provided herein or any embodiment thereof. All patents, patent applications, and publications cited herein are fully incorporated by reference herein in their entirety.

What is claimed is:

1. A compound having Formula (I): 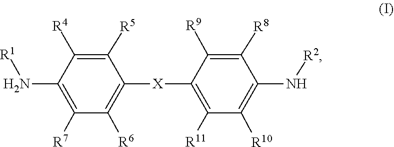
or a salt or solvate thereof, wherein:
R¹ is selected from the group consisting of C₁-C₁₂ alkyl, —CHR^{1a}R^{1b}, C₃-C₆ cycloalkyl, and optionally substituted phenyl; or
R¹ and R⁴ taken together with the nitrogen and carbon atoms, respectively, to which they are attached form a saturated or partially saturated optionally substituted 5- or 6-membered heterocyclo;
R^{1a} is selected from the group consisting of optionally substituted phenyl, C₃-C₆ cycloalkyl, and optionally substituted 5- or 6-membered heteroaryl;
R^{1b} is selected from the group consisting of hydrogen and C₁-C₆ alkyl;
R² is selected from the group consisting of C₁-C₁₂ alkyl, —CHR^{2a}R^{2b}, C₃-C₆ cycloalkyl, and optionally substituted phenyl; or
R² and R⁸ taken together with the nitrogen and carbon atoms, respectively, to which they are attached form a saturated or partially saturated optionally substituted 5- or 6-membered heterocyclo;
R^{2a} is selected from the group consisting of optionally substituted phenyl, C₃-C₆ cycloalkyl, and optionally substituted 5- or 6-membered heteroaryl;
R^{2b} is selected from the group consisting of hydrogen and C₁-C₆ alkyl;
X is selected from the group consisting of —CHR^{3a}—, —NR^{3b}—, —O—, and —S—;
R^{3a} is selected from the group consisting of hydrogen, C₁-C₁₂ alkyl, C₂-C₆ alkenyl, C₃-C₆ cycloalkyl, optionally substituted phenyl, optionally substituted 5- or 6-membered heterocyclo, and optionally substituted 5- or 6-membered heteroaryl;
R^{3b} is selected from the group consisting of hydrogen, C₁-C₁₂ alkyl, C₃-C₆ cycloalkyl, optionally substituted phenyl, optionally substituted 5- or 6-membered heterocyclo, and optionally substituted 5- or 6-membered heteroaryl;
R⁴, R⁵, R⁶, and R⁷ are independently selected from the group consisting of hydrogen, halogen, C₁-C₆ alkyl, C₁-C₆ alkoxy, and C₁-C₆ alkylthio; or
R¹ and R⁴ taken together with the nitrogen and carbon atoms, respectively, to which they are attached form a saturated or partially saturated optionally substituted 5- or 6-membered heterocyclo; and
R⁵, R⁶, and R⁷ are independently selected from the group consisting of hydrogen, halogen, C₁-C₆ alkyl, C₁-C₆ alkoxy, and C₁-C₆ alkylthio; and
R⁸, R⁹, R¹⁰, and R¹¹ are independently selected from the group consisting of hydrogen, halogen, C₁-C₆ alkyl, C₁-C₆ alkoxy, and C₁-C₆ alkylthio; or
R² and R⁸ taken together with the nitrogen and carbon atoms, respectively, to which they are attached form a saturated or partially saturated optionally substituted 5- or 6-membered heterocyclo; and
R⁹, R¹⁰, and R¹¹ are independently selected from the group consisting of hydrogen, halogen, C₁-C₆ alkyl, C₁-C₆ alkoxy, and C₁-C₆ alkylthio.

2. The compound of claim 1, or a salt or solvate thereof, wherein X is —NR^{3b}—.

3. The compound of claim 1, or a salt or solvate thereof, wherein X is —O—.

4. The compound of claim 1, or a salt or solvate thereof, wherein X is —S—.

5. The compound of claim 1, or a salt or solvate thereof, wherein X is —CHR^{3a}—.

6. The compound of claim 5 having Formula (II): 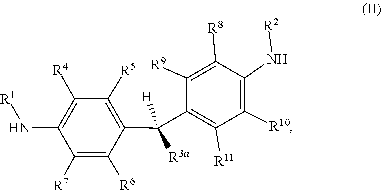
or a salt or solvate thereof, wherein R^{3a} is selected from the group consisting of C₁-C₁₂ alkyl, C₂-C₆ alkenyl, C₃-C₆ cycloalkyl, optionally substituted phenyl, and optionally substituted 5- or 6-membered heteroaryl,
with the proviso that 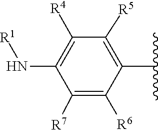
does not equal

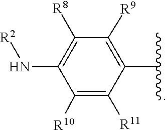

7. The compound of claim 5 having Formula (III): 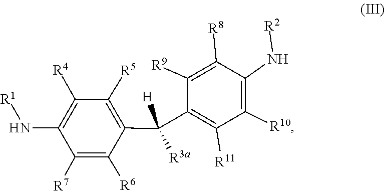
or a salt or solvate thereof, wherein R^{3a} is selected from the group consisting of C₁-C₁₂ alkyl, C₂-C₆ alkenyl, C₃-C₆ cycloalkyl, optionally substituted phenyl, and optionally substituted 5- or 6-membered heteroaryl,
with the proviso that 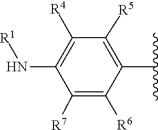
does not equal

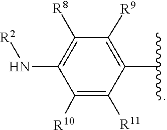

8. The compound of any one of claims 1-7, or a salt or solvate thereof, wherein R¹ and R² are independently selected from the group consisting of methyl, ethyl, propyl, isopropyl, butyl, iso-butyl, sec-butyl, tert-butyl, pentyl, iso-pentyl, hexyl, heptyl, octyl, nonyl, decyl, undecyl, dodecyl, and optionally substituted phenyl.

9. The compound of claim 8, or a salt or solvate thereof, wherein R¹ and R² are iso-propyl.

10. The compound of any one of claims 1-9, or a salt or solvate thereof, wherein R⁴, R⁵, R⁶, R⁷, R⁸, R⁹, R¹⁰, and R¹¹ are selected from the group consisting of hydrogen and methyl.

11. The compound of claim 10, or a salt or solvate thereof, wherein R⁴, R⁵, R⁶, R⁷, R⁸, R⁹, R¹⁰, and R¹¹ are hydrogen.

12. The compound of any one of claims 1 or 5-11, or a salt or solvate thereof, wherein:
R^{3a} is selected from the group consisting of C₁-C₁₂ alkyl, 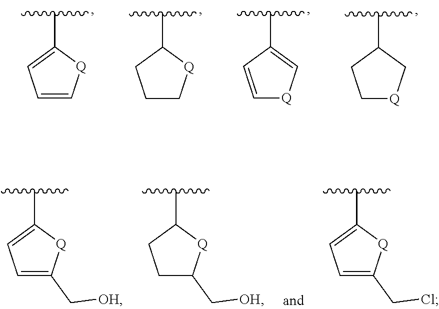
and
Q is —NH—, —O—, or —S—.

13. The compound of claim 12, or a salt or solvate thereof, wherein R^{3a} is C₁-C₁₂ alkyl.

14. The compound of claim 13, or a salt or solvate thereof, wherein R^{3a} is methyl, ethyl, propyl, isopropyl, butyl, iso-butyl, sec-butyl, or tert-butyl.

15. The compound of claim 12, or a salt or solvate thereof, wherein R^{3a} is

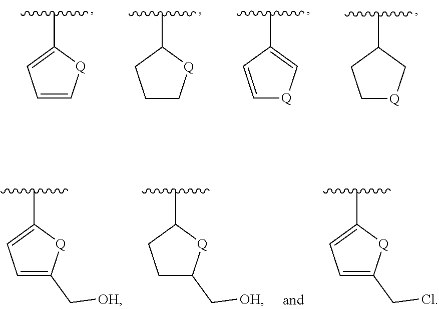

16. The compound of claim 15, or a salt or solvate thereof, wherein Q is —O—.

17. The compound of claim 5, or a salt or solvate thereof, having Formula (IV): 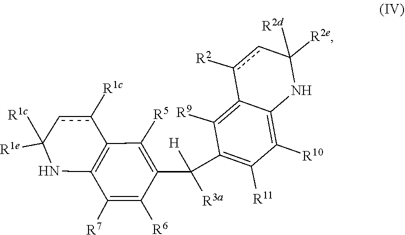
or a salt or solvate thereof, wherein:
each [symbol] is a single or double bond; and
R^{1c}, R^{1d}, R^{1e}, R^{2c}, R^{2d}, and R^{2e} are independently selected from the group consisting of hydrogen and C₁-C₆ alkyl.

18. The compound of claim 5, or a salt or solvate thereof, having Formula (IVa): 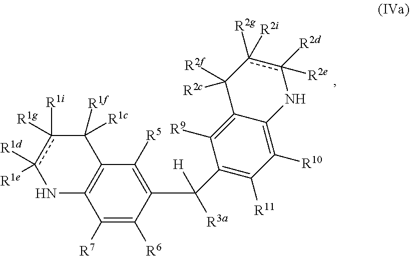
or a salt or solvate thereof, wherein:
each [symbol] is a single or double bond;
R^{1c}, R^{1d}, R^{1e}, R^{2c}, R^{2d}, and R^{2e} are independently selected from the group consisting of hydrogen, C₁-C₉ alkyl, C₃-C₆ cycloalkyl, and optionally substituted phenyl;
R^{1f} and R^{2f} are independently selected from the group consisting of hydrogen, halogen, C₁-C₉ alkyl, C₁-C₉ haloalkyl, C₃-C₈ cycloalkyl, —OH, —SH, C₁-C₈ alkoxy, C₁-C₈ alkylthio, arylthio, —C(═O)OR, —OC(═O)R, —C(═O)NRR′, —NRC(═O)R′, —NRR′, —SC(═O)R, —SC(═O)SR, —SC(═O)NRR′, —NRC(═O)SR′, —NRC(═S)SR′, 4- to 6-membered heterocyclyl, optionally substituted phenyl, and optionally substituted 5- or 6-membered heteroaryl;
R and R′ are at each occurrence independently selected from the group consisting of hydrogen, C₁-C₉ alkyl, C₃-C₆ cycloalkyl, and optionally substituted phenyl; and
R^{1g}, R^{2g}, R¹ⁱ, and R²ⁱ are independently selected from the group consisting of hydrogen, C₁-C₉ alkyl, C₃-C₆ cycloalkyl, and optionally substituted phenyl,
with the proviso that if [symbol] linking the carbons attached to R^{1e} and R¹ⁱ is a double bond, R^{1e} and R¹ⁱ are absent, and if the [symbol] linking the carbons attached to R^{2e} and R²ⁱ is a double bond, R^{2e} and R²ⁱ are absent.

19. The compound of claim 17, or a salt or solvate thereof, having Formula (V): 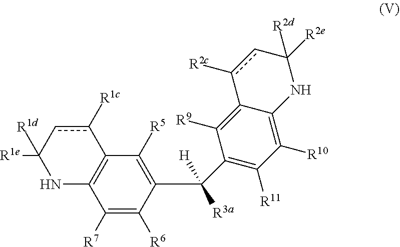
or a salt or solvate thereof,
with the proviso that 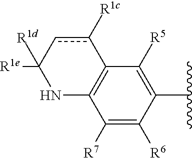
does not equal

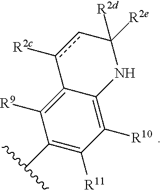

20. The compound of claim 18, or a salt or solvate thereof, having Formula (Va): 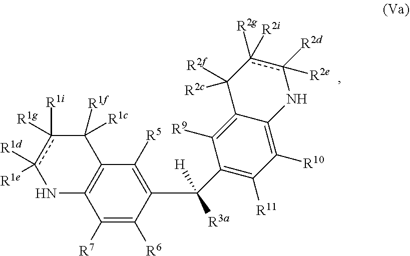
or a salt or solvate thereof,
with the proviso that 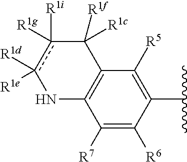
does not equal

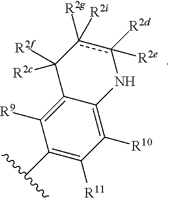

21. The compound of claim 17, or a salt or solvate thereof having Formula (VI): 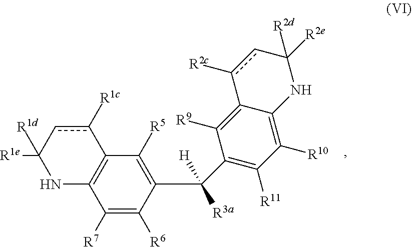
or a salt or solvate thereof,
with the proviso that 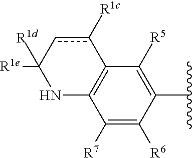
does not equal

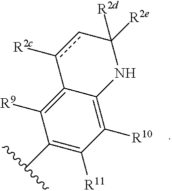

22. The compound of claim 18, or a salt or solvate thereof, having Formula (VIa): 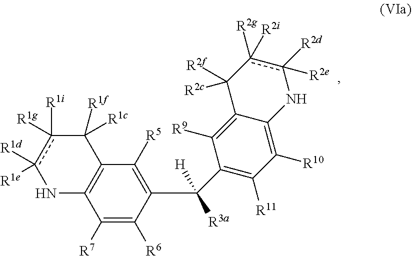
or a salt or solvate thereof,
with the proviso that 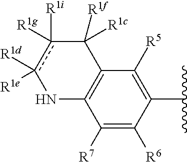
does not equal

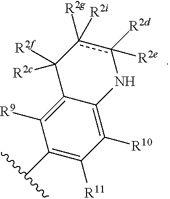

23. The compound of any one of claims 17-22, or a salt or solvate thereof, wherein R⁵, R⁶, R⁷, R⁹, R¹⁰, and R¹¹ are selected from the group consisting of hydrogen, methyl, C₁-C₆ alkoxy, and C₁-C₆ alkylthio.

24. The compound of claim 23, or a salt or solvate thereof, wherein R⁵, R⁶, R⁷, R⁹, R¹⁰, and R¹¹ are hydrogen.

25. The compound of any one of claims 17-24, or a salt or solvate thereof, wherein R^{1c}, R^{1d}, R^{1e}, R^{2c}, R^{2d}, and R^{2e} are methyl, ethyl, or hydrogen.

26. The compound of claim 25, or a salt or solvate thereof, wherein R^{1c}, R^{1d}, R^{1e}, R^{2c}, R^{2d}, and R^{2e} are methyl.

27. The compound of any one of claims 17, 18 or 23-26, or a salt or solvate thereof, wherein R^{3a} is hydrogen.

28. The compound of any one of claims 17-26, or a salt or solvate thereof, wherein:
R^{3a} is selected from the group consisting of C₁-C₁₂ alkyl, 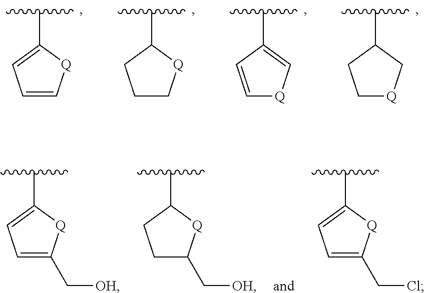
and
Q is —NH—, —O—, or —S—.

29. The compound of claim 28, or a salt or solvate thereof, wherein R^{3a} is C₁-C₁₂ alkyl.

30. The compound of claim 29, or a salt or solvate thereof, wherein R^{3a} is methyl, ethyl, propyl, isopropyl, butyl, iso-butyl, sec-butyl, or tert-butyl.

31. The compound of claim 28, or a salt or solvate thereof, wherein R^{3a} is:

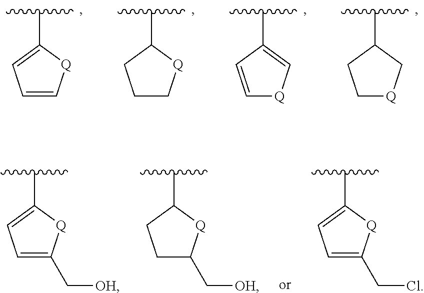

32. The compound of claim 31, or a salt or solvate thereof, wherein Q is —O—.

33. The compound of claim 1, or a salt or solvate thereof selected from any one or more of the compounds of Table 1.

34. A composition comprising:
(i) the compound of any one of claims 1-33; and
(ii) one or more elastomers; or
(iii) one or more fillers; or
(iv) one or more rubber chemicals; or
(v) one or more plasticizers; or
(vi) a second antidegradant; or
(vii) a combination of one or more elastomers, one or more fillers, one or more rubber chemicals, one or more plasticizers, and/or a second antidegradant.

35. The composition of claim 34, wherein the composition comprises one or more elastomers.

36. The composition of claim 35, wherein the composition comprises from about 15 wt/wt % to about 85 wt/wt % of the compound and from about 15 wt/wt % to about 85 wt/wt % of the one or more elastomers.

37. The composition of any one of claims 34-37, wherein the one or more elastomers comprise natural rubber (NR).

38. The composition of claim 37, wherein the one or more elastomers comprises from about 5 wt/wt % to about 80 wt/wt % natural rubber (NR).

39. The composition of claim 37 or 38, wherein the natural rubber comprises rubber derived from an alternative rubber plant.

40. The composition of claim 39, wherein the alternative rubber plant is Parthenium argentatum (guayule) or Taraxacum kok-saghyz (Russian dandelion).

41. The composition of any one of claims 35-40, wherein the one or more elastomers comprise synthetic rubber.

42. The composition of claim 41, wherein the synthetic rubber comprises an unsaturated rubber, a saturated rubber, a rubber with fluoro and fluoralkyl or fluoralkoxy substituent groups on the polymer chain (FKM), a silicone rubber (Q), or a blend thereof.

43. The composition of claim 42, wherein the unsaturated rubber comprises polyisoprene rubber (IR), butyl rubber (IIR), polybutadiene rubber (BR), styrene butadiene rubber (SBR), nitrile butadiene rubber (NBR), chloroprene rubber (CR), ethylene propylene diene rubber (EPDM), or a blend thereof.

44. The composition of claim 43, wherein the unsaturated rubber comprises styrene butadiene rubber (SBR).

45. The composition of claim 44, wherein the one or more elastomers comprise from about 5 wt/wt % to about 80 wt/wt % styrene butadiene rubber (SBR).

46. The composition of any one of claims 43-45, wherein the unsaturated rubber comprises polybutadiene rubber (BR).

47. The composition of claim 46, wherein the one or more elastomers comprise from about 5 wt/wt % to about 80 wt/wt % polybutadiene rubber (BR).

48. The composition of any one of claims 43-47, wherein the unsaturated rubber comprises butyl rubber (IIR).

49. The composition of claim 48, wherein the one or more elastomers comprise from about 1 wt/wt % to about 30 wt/wt % butyl rubber (IIR).

50. The composition of any one of claims 42-49, wherein the saturated rubber comprises acrylic rubber (ACM), chlorinated polyethylene (CM), chlorosulfonated polyethylene (CSM), polychloromethyloxiran (CO), ethylene-ethyl acrylate copolymer (EAM), epichlorohydrin rubber (ECO), ethylene propylene rubber (EPM), ethylenevinylacetate copolymer (EVM), or a blend thereof.

51. The composition of claims any one of claims 34-50, wherein the one or more elastomers further comprises recycled rubber.

52. The composition of any one of claims 34-51, wherein the composition comprises from about 0.1 phr to about 10 phr of the compound.

53. The composition of claim 52, wherein the composition comprises from about 0.5 phr to about 5 phr of the compound.

54. The compound of claim 53, wherein the composition comprises from about 1 phr to about 5 phr of the compound.

55. The composition of any one of claims 34-54, wherein the composition comprises one or more fillers.

56. The composition of claim 55, wherein the composition comprises from about 15 wt/wt % to about 85 wt/wt % of the compound and from about 15 wt/wt % to about 85 wt/wt % of the one or more fillers.

57. The composition of claims 55 or 56, wherein the composition comprises from about 30 phr to about 500 phr of one or more fillers.

58. The composition of any one of claims 55-57, wherein the one or more fillers comprise carbon black, silica, kaolin, calcium silicate, talc, carbon nanotubes (CNT), carbon fibers (HCF), graphite, graphenes, aluminosilicates, starch, fibers, or a combination thereof.

59. The composition of claim 58, wherein the one or more fillers comprise silica.

60. The composition of claim 59, wherein the silica is derived from rice husks.

61. The composition of any one of claims 54-60, wherein the one or more fillers comprise carbon black.

62. The composition of any one of claims claim 34-61, wherein the composition comprises one or more rubber chemicals.

63. The composition of claim 62, wherein the composition comprises from about 15 wt/wt % to about 85 wt/wt % of the compound and from about 15 wt/wt % to about 85 wt/wt % of one or more rubber chemicals.

64. The composition of claims 62 or 63, wherein the composition comprises from about 0.1 phr to about 30 phr of one or more rubber chemicals.

65. The composition of claim 64, wherein the composition comprises from about 1 phr to about 20 phr of one or more rubber chemicals.

66. The composition of any one of claims 62-65, wherein the one or more rubber chemicals comprise one or more vulcanizing agents, one or more accelerators, one or more activators, one or more pre-vulcanization inhibitors, or a combination thereof.

67. The composition of claim 66, wherein the one or more rubber chemicals comprise one or more vulcanizing agents.

68. The composition of claim 67, wherein the one or more vulcanizing agents comprise sulfur, peroxide, resin, or a combination thereof.

69. The composition of claim 68, wherein the sulfur is octasulfur (S₈), cyclododecasulfur (S₁₂), polymeric sulfur, or a combination thereof.

70. The composition of claim 68, wherein the peroxide is benzoyl peroxide, dicumyl peroxide (DC), 2,5-dimethyl-2,5-di-(tert-butylperoxy)-3-hexyne (2,5 Tri), 2,5-dimethyl-2,5-di(tert-butylperoxy)hexane (DDPH), di-(2-tert-butylperoxyisopropyl)benzene (VC), butyl-4,4-di-(tert-butylperoxy)valerate (VAL), 1,1-di(tert-butylperoxy)-3,3,5-trimethylcyclohexane (TMC), or a combination thereof.

71. The composition of claim 68, wherein the resin is a bonding resin.

72. The composition of claims 66-71, wherein the one or more rubber chemicals comprise one or more accelerators.

73. The composition of claim 72, wherein the one or more accelerators comprise a guanidine, a thiazole, a sulfenamide, a thiuram, a dithiocarbamate, a xanthate, a thiophosphate, or a combination thereof.

74. The composition of claim 73, wherein the guanidine is diphenylguanidine (DPG).

75. The composition of claim 73, wherein the thiazole comprises 2-mercaptobenzothiazole (MBT), zinc 2-mercaptobenzothiazole (ZMBT), mercaptobenzothiazole disulfide (MBTS), N-tert-butyl-2-benzothiazole sulfenimide (TBSI), or a combination thereof.

76. The composition of claim 73, wherein the sulfenamide comprises N-tert-butyl-2-benzothiazylsulfenamide (TBBS), N-cyclohexylbenzothiazol-2-sulfenamide (CBS), dicyclohexyl-2-benzothiazolesulfenamide (DCBS), N-oxydiethylene benzothiazole sulfenamide (OBTS), N-oxydiethylenethiocarbamyl-N′-oxydiethylene sulfenamide (OTOS), thiocarbamyl sulfenamide, or a combination thereof.

77. The composition of claim 73, wherein the thiuram is dimethylcarbamothioic dithioperoxyanhydride (thiram), dipentamethylene thiuram tetrasulfide (DPIT), tetrabenzyl thiuram disulfide (TBzTD), tetraethylthiuram disulfide (TETD), tetramethylthiuram disulfide (TMTD), tetramethylthiuram monosulfide (TMTM), or a combination thereof.

78. The composition of claim 73, wherein the dithiocarbamate comprises zinc dimethyldithiocarbamate (ZDMC), zinc diethyldithiocarbamate (ZDEC), zinc dibutyldithiocarbamate (ZDBC), nickel dibutyldithiocarbamate (NDBC), sodium dibenzyldithiocarbamate (SBEC), sodium diethyldithiocarbamate (SDEC), tellurium diethyldithiocarbamate (TDEC), zinc dibenzyldithiocarbamate (ZEBC), or a combination thereof.

79. The composition of any one of claims 66-78, wherein the one or more rubber chemicals comprise one or more activators.

80. The composition of claim 79, wherein the one or more activators comprise a metal oxide, an acid, a metal complex, or a combination thereof.

81. The composition of claim 80, wherein the metal oxide comprises zinc oxide, magnesium oxide, lead oxide, or a combination thereof.

82. The composition of claim 80, wherein the acid comprises stearic acid, lauric acid, or a combination thereof.

83. The composition of claim 80, wherein the metal complex comprises zinc ethylhexanoate.

84. The composition of any one of claims 66-83, wherein the one or more rubber chemicals comprise one or more pre-vulcanization inhibitors.

85. The composition of claim 84, wherein the one or more pre-vulcanization inhibitors comprise N-(cyclohexylthio)phthalimide (CTP), benzoic anhydride, salicylic anhydride, phthalic anhydride, or a combination thereof.

86. The composition of any one of claims 34-85, wherein the composition comprises one or more plasticizers.

87. The composition of claim 86, wherein the composition comprises from about 15 wt/wt % to about 85 wt/wt % of the compound and from about 15 wt/wt % to about 85 wt/wt % of one or more plasticizers.

88. The composition of claims 86 or 87, wherein the composition comprises from about 0.1 phr to about 30 phr of one or more plasticizers.

89. The composition of claim 88, wherein the composition comprises from about 1 phr to about 20 phr of one or more plasticizers.

90. The composition of any one of claims 86-89, wherein the one or more plasticizers comprise a mineral oil, an organic ester, a resin, a wax, an ester plasticizer, a naturally derived oil, or a combination thereof.

91. The composition of claim 90, wherein the mineral oil is naphthenic oil, paraffinic oil, or aromatic oil.

92. The composition of claim 90, wherein the naturally derived oil is soybean oil, vegetable oil, or orange oil.

93. The composition of any one of claims 34-92, wherein the composition further comprises a second antidegradant.

94. The composition of claim 93, wherein the composition comprises from about 15 wt/wt % to about 85 wt/wt % of the compound and from about 15 wt/wt % to about 85 wt/wt % of the second antidegradant.

95. The composition of claim 93 or 94, wherein the second antidegradant is present in an amount of from about 0.001 phr to about 10 phr.

96. The composition of claim 95, wherein the second antidegradant is present in an amount of from about 0.1 phr to about 5 phr.

97. The composition of claim 96, wherein the second antidegradant is present in an amount of from about 0.5 phr to about 5 phr.

98. The composition of claim 97, wherein the second antidegradant is present in an amount of from about 1 phr to about 5 phr.

99. The composition of any one of claims 93-98, wherein the second antidegradant is an antioxidant.

100. The composition of any one of claims 93-98, wherein the second antidegradant is an antiozonant.

101. The composition of any one of claims 93-100, wherein the second antidegradant is a paraphenylenediamine (PPD), a trimethyldihydroquinoline (TMQ), a phenolic, an alkylated diphenylamine (DPA), or a diphenylamine-ketone condensate.

102. The composition of claim 101, wherein the PPD is N¹-(4-methylpentan-2-yl)-N⁴-phenylbenzene-1,4-diamine (6PPD), N-(1,4-dimethylpentyl)-N′-phenyl-p-phenylenediamine (7PPD), N¹-phenyl-N⁴-(propan-2-yl)benzene-1,4-diamine (IPPD), N,N′-di-sec-butyl-p-phenylenediamine (44PD), N,N′-bis(1,3-dimethylbutyl)-p-phenylenediamine (66PD), N,N′-bis(1,4-dimethylpentyl)-p-phenylenediamine (77PD), or N—N′-dioctyl-p-phenylenediamine (88PD).

103. The composition of claim 102, wherein the PPD is 6PPD.

104. The composition of claim 101, wherein the TMQ is 2,2,4-trimethyl-1,2-dihydroquinoline or an oligomer or polymer thereof.

105. A composition comprising the compound of any one of claims 1-33 and one or more carriers.

106. A process for preparing the composition of claim 105, the process comprising admixing the compound and the one or more carriers.

107. A vulcanized elastomeric article comprising the compound of any one of claims 1-33.

108. A vulcanized elastomeric article prepared using the composition of any one of claims 34-105.

109. The vulcanized elastomeric article of claims 107 or 108, wherein the vulcanized elastomeric article is a tire.

110. The vulcanized elastomeric article of claim 109, wherein the tire is a passenger vehicle tire, a light truck tire, a heavy truck or bus tire, a motorcycle tire, an agriculture tire, an earthmover tire, an airplane tire, or a racing tire.

111. The vulcanized elastomeric article of claims 107 or 108, wherein the vulcanized elastomeric article is a component of a tire.

112. The vulcanized elastomeric article of claim 111, wherein the component is a bead, a belt, a body ply, an inner liner, a sidewall, an undertread, or a tread.

113. The vulcanized elastomeric article of claims 107 or 108, wherein the vulcanized elastomeric article is a rubber overshoe, a sealing strip, an acoustic panel, an air spring, a bellow, a membrane, a tactile sensor, a crash pad, a hose, a conveyor belt, or a flooring.

114. A process for preparing a vulcanized elastomeric article, the process comprising:
(a) forming the composition of any one of claims 33-105 into a formed shape; and
(b) vulcanizing the formed shape
to provide a vulcanized elastomeric article.

115. The process of claim 114, wherein the vulcanizing is performed at an average temperature of from about 120° C. to about 180° C.

116. The process of claim 115, wherein the vulcanizing is performed at an average temperature of from about 140° C. to about 160° C.

117. The process of any one of claims 114-116, wherein the vulcanized elastomeric article is a tire.

118. The process of claim 117, wherein the tire is a passenger vehicle tire, a light truck tire, a heavy truck or bus tire, a motorcycle tire, an agriculture tire, an earthmover tire, an airplane tire, or a racing tire.

119. The process of any one of claims 114-118, wherein the vulcanized elastomeric article is a component of a tire.

120. The process of claim 119, wherein the component is a bead, a belt, a body ply, an inner liner, a sidewall, an undertread, or a tread.

121. The process of any one of claims 114-116, wherein the vulcanized elastomeric article is a rubber overshoe, a sealing strip, an acoustic panel, an air spring, a bellow, a membrane, a tactile sensor, a crash pad, a hose, a conveyor belt, or a flooring.

122. A lubricant composition comprising a lubricant and the compound of any one of claims 1-33.

123. A combustible fuel composition comprising a combustible fuel and the compound of any one of claims 1-33.

124. A fuel additive composition comprising a fuel additive and the compound of any one of claims 1-33.

125. A process for retreading tires, the process comprising:
(a) applying the composition of any one of claims 34-105 to a tire;
(b) disposing a pre-vulcanized tread around the tire;
(c) disposing a curing envelope around the tire; and
(d) vulcanizing the tire.

126. A kit comprising the composition of any one of claims 34-105 and instructions for using the composition in a vulcanizable elastomeric composition.

127. A kit comprising the composition of any one of claims 34-105 and instructions for using the composition to prepare a vulcanized elastomeric article.

128. The composition of claim 34, wherein the composition comprises from about 0.1 wt/wt % to about 10 wt/wt % of the compound.
